(19) United States
(12) Patent Application Publication
(10) Pub. No.: US 2005/0245557 A1
(43) Pub. Date: Nov. 3, 2005

(54) Methods and materials useful for the treatment of arthritic conditions, inflammation associated with a chronic condition or chronic pain

(75) Inventors: Grant L. Schoenhard, San Carlos, CA (US); Nadav Friedmann, Lafayette, CA (US)

Correspondence Address: MCANDREWS HELD & MALLOY, LTD, 500 WEST MADISON STREET, SUITE 3400, CHICAGO, IL, 60661, US

(73) Assignee: Pain Therapeutics, Inc.

(21) Appl. No.: 10/966,703

(22) Filed: Oct. 15, 2004

Related U.S. Application Data
(60) Provisional application No. 60/511,841, filed on Oct. 15, 2003.
(60) Provisional application No. 60/566,189, filed on Apr. 27, 2004.

(51) Int. Cl. A61K 31/485

(52) U.S. Cl. 514/282

(57) ABSTRACT

Methods and materials, including novel compositions, dosage forms and methods of administration, useful for treating arthritic conditions, inflammation associated with a chronic condition, and/or chronic pain, including pain from arthritis and inflammation, using opioid antagonists, including combinations of opioid antagonists and opioid agonists. Methods and materials comprising opioid antagonists or combinations opioid antagonists and agonists may optionally include one or more additional therapeutic agents.

### CROSS REFERENCE TO RELATED APPLICATIONS

This application claims the priority of the following U.S. Patent Application No. 60/511,841, filed Oct. 15, 2003 (provisional) and U.S. Patent Application No. 60/566,189, filed Apr. 27, 2004 (provisional). The applications cited above are hereby incorporated herein by reference in their entirety.

### FIELD OF THE INVENTION

The present invention relates to methods and materials, including novel compositions, dosage forms and methods of administration, useful for the treatment of arthritic conditions, inflammation associated with a chronic condition, and/or chronic pain, including pain from arthritic conditions or inflammation, using opioid antagonists, including combinations of opioid antagonists and opioid agonists. The methods and materials provide human subjects with an alleviation of one or more symptoms or signs of an arthritic condition, inflammation associated with a chronic condition or chronic pain, including, for example, reduced pain, reduced stiffness and/or improved physical function. Methods and materials of the invention comprising opioid antagonists or combinations of opioid antagonists and agonists may optionally include one or more additional therapeutic agents.

### BACKGROUND OF THE INVENTION

The inflammatory process involves a series of events that can be elicited by numerous stimuli (e.g., infectious agents, ischemia, antigen-antibody interactions, and thermal or other physical injury). At a macroscopic level, inflammation usually is accompanied by the familiar clinical signs of erythema, edema, tenderness (hyperalgesia), and pain. Inflammatory responses occur in three distinct phases, each apparently mediated by different mechanisms: (1) an acute transient phase, characterized by local vasodilation and increased capillary permeability; (2) a delayed, subacute phase, most prominently characterized by infiltration of leukocytes and phagocytic cells; and (3) a chronic proliferative phase, in which tissue degeneration and fibrosis occur. Many different mechanisms are involved in inflammation (see, e.g., Gallin et al., eds. INFLAMMATION: BASIC PRINCIPLES AND CLINICAL CORRELATES (2nd ed., 1992); Kelly et al., eds. TEXTBOOK OF RHEUMATOLOGY (4th ed. 1993)). The ability to mount an inflammatory response is essential for survival in the face of environmental pathogens and injury, although in some situations and diseases the inflammatory response may be exaggerated and sustained for no apparent beneficial reason. Thus, while inflammation associated with acute conditions of infection or injury may be beneficial, inflammation associated with chronic conditions may be destructive.

Inflammation arises in connection with many chronic conditions, including, for example, arthritic conditions. One type of arthritic condition is osteoarthritis (OA). Osteoarthritis is a degenerative joint disease, characterized by the breakdown of the joint's cartilage. Cartilage breakdown causes bones to rub against each other, causing pain and/or loss of movement. Most commonly affecting middle-aged and older people, osteoarthritis can range from very mild to very severe. It affects hands and weight-bearing joints such as knees, hips, feet, the back and/or the neck.

Another chronic condition with which inflammation is associated is rheumatoid arthritis (RA). Rheumatoid arthritis involves inflammation in the lining of the joints and/or other internal organs. Rheumatoid arthritis typically affects many different joints. It is typically chronic, but can be a disease of flare-ups. Rheumatoid arthritis is a systemic disease that affects the entire body and is one of the most common forms of arthritis. It is characterized by the inflammation of the membrane lining the joint (the synovium), which causes pain, stiffness, warmth, redness and/or swelling. The inflamed synovium can invade and damage bone and cartilage. Inflammatory cells release enzymes that may digest bone and cartilage. The involved joint can lose its shape and alignment, resulting in pain and loss of movement.

Yet another chronic condition with which inflammation is associated is back pain, particularly lower back pain. Lower back pain affects approximately two-thirds of the U.S. adult population, leads to significant increases in physician office visits, and has a significant effect on disability.

Prostaglandins are recognized as participating in the inflammatory process. Prostaglandins are released whenever cells are damaged, they appear in inflammatory exudates. Several classes of leukocytes play roles in inflammation. Several different cytokines also appear to play roles in the inflammatory process, especially interleukin 1 (IL-1) and tumor necrosis factor (TNF). IL-1 and TNF appear to work in concert with each other and with growth factors (such as granulocyte/macrophage colony stimulating factor, GM-CSF) and other cytokines, such as IL-8 and related chemotactic cytokines (chemokines), which can promote neutrophil infiltration and activation. TNF is composed of two closely related proteins: mature TNF (TNFα) and lymphotoxin (TNFβ). Other cytokines and growth factors (e.g., IL-2, IL-6, IL-8, and GM-CSF) contribute to manifestations of the inflammatory response. The concentrations of many of these factors are increased in the synovia of patients with arthritides, such as rheumatoid arthritis. The concentration of peptides, such as substance P, which promotes firing of pain fibers, also is increased at such sites.

To counter the effects of proinflammatory mediators, other cytokines and growth factors have been implicated as having anti-inflammatory activity. These include transforming growth factor-β₁ (TGF-β₁, which increases extracellular matrix formation but also acts as an immunosuppressant), interleukin 10 (IL-10, which has inhibitory effects on monocytes, including decreased cytokine and prostaglandin E₂ formation), and interferon gamma (IFN-γ, which possesses myelosuppressive activity and inhibits collagen synthesis and collagenase production by macrophages).

Histamine is also a mediator of the inflammatory process. Although several H₁ histamine-receptor antagonists are available, they are useful only for the treatment of vascular events in the early transient phase of inflammation. Bradykinin and 5-hydroxytryptamine (serotonin, 5-HT) also may play a role in mediating inflammation, but their antagonists ameliorate only certain types of inflammatory responses. Specific inhibitors of leukotriene synthesis, (zileuton, a 5-lipoxygenase inhibitor) and cysteinyl leukotriene-receptor antagonists (montelukast and zafirlukast) exert anti-inflammatory actions and have been approved for the treatment of asthma. Another lipid autacoid, platelet-activating factor (PAF), has been implicated as an important mediator of inflammation, and inhibitors of its synthesis and action are under study.

Although the pathogenesis of rheumatoid arthritis is largely unknown, it appears to be an autoimmune disease driven primarily by activated T cells, giving rise to T cell-derived cytokines, such as IL-1 and TNF. Although activation of B cells and the humoral response also are evident, most of the antibodies generated are IgG of unknown specificity, apparently elicited by polyclonal activation of B cells rather than from a response to a specific antigen.

Many cytokines, including IL-1 and TNF, have been found in the rheumatoid synovium. Of the available anti-inflammatory drugs, only the adrenocorticosteroids are known to interfere with the synthesis and/or actions of cytokines such as IL-1 or TNF. Although some of the actions of these cytokines are accompanied by the release of prostaglandins and/or thromboxane A₂, only their pyrogenic effects are blocked by inhibitors of cyclooxygenase. In addition, many of the actions of the prostaglandins are inhibitory to the immune response, including suppression of the function of helper T cells and B cells and inhibition of the production of IL-1. Thus, it is difficult to ascribe the anti-rheumatoid effects of aspirin-like drugs solely to inhibition of prostaglandin synthesis.

Bradykinin, released from plasma kininogen, and cytokines, such as TNF-α, IL-1, and IL-8, appear to be particularly important in eliciting the pain associated with inflammation. These agents liberate prostaglandins and probably other mediators that promote hyperalgesia. Neuropeptides, such as substance P and calcitonin gene-related peptide, also may be involved in eliciting pain.

Nonsteroidal anti-inflammatory drugs (NSAIDs) are known and prescribed for their anti-inflammatory, antipyretic, and analgesic effects. NSAIDs are known to inhibit the biosynthesis of prostaglandins. However, NSAIDs generally do not inhibit the formation of eicosanoids such as the leukotrienes, which also contribute to inflammation, nor do they affect the synthesis of numerous other inflammatory mediators. NSAIDs may have other actions that contribute to their therapeutic effects.

While most NSAIDs are antipyretic, analgesic, and anti-inflammatory, an important exception is acetaminophen, which is antipyretic and analgesic but is largely devoid of anti-inflammatory activity. This can be explained by the fact that acetaminophen effectively inhibits cyclooxygenases in the brain but not at sites of inflammation in peripheral tissues.

Aspirin is a known NSAID that covalently modifies both cyclooxygenase-1 (COX-1) and cyclooxygenase-2 (COX-2) primarily via inhibition, thus resulting in an irreversible inhibition of cyclooxygenase activity. The vast majority of NSAIDs are organic acids and, in contrast to aspirin, act as reversible, competitive inhibitors of cyclooxygenase activity.

CELEBREX® (celecoxib) is a nonsteroidal anti-inflammatory drug that exhibits anti-inflammatory, analgesic, and antipyretic activities. The mechanism of action of CELEBREX® is believed to be due to inhibition of prostaglandin synthesis, primarily via inhibition of COX-2, and at therapeutic concentrations in humans, CELEBREX® does not inhibit COX-1. CELEBREX® is chemically designated as 4-[5-(4-methylphenyl)-3-(trifluoromethyl)-1H-pyrazol-1-yl] benzene-sulfonamide and is a diaryl substituted pyrazole.

BEXTRA® (valdecoxib) is a nonsteroidal anti-inflammatory drug that exhibits anti-inflammatory, analgesic, and antipyretic activities. The mechanism of action is believed to be due to inhibition of prostaglandin synthesis primarily through inhibition of COX-2. BEXTRA® (valdecoxib) is chemically designated as 4-(5-methyl-8-phenyl-4-isoxazolyl) benzenesulfonamide and is a diaryl substituted is oxazole.

CELEBREX® and BEXTRA® are used to treat osteoarthritis and are said to demonstrate significant reduction in joint pain compared to placebo. CELEBREX® and BEXTRA® are also used to treat rheumatoid arthritis and are said to demonstrate significant reduction in joint tenderness/pain and joint swelling compared to placebo. CELEBREX® and BEXTRA® share many of the side effects of other NSAIDs and can cause discomfort and (relatively rarely) more serious side effects, such as gastrointestinal bleeding.

NSAIDs such as aspirin, acetaminophen, CELEBREX®, and BEXTRA®, find clinical application as anti-inflammatory agents in the treatment of musculoskeletal disorders, such as rheumatoid arthritis, osteoarthritis, and ankylosing spondylitis. Chronic treatment of patients with rofecoxib and celecoxib has been shown to be effective in suppressing inflammation without the gastric toxicity that is associated with treatment with nonselective NSAIDS. In general, NSAIDs provide only symptomatic relief from the pain and inflammation associated with the disease and do not arrest the progression of pathological injury to tissue.

In addition to sharing many therapeutic activities, NSAIDs share several unwanted side effects. The most common is a risk of gastric or intestinal ulceration that sometimes can be accompanied by anemia from the resultant blood loss, though the selective COX-2 inhibitors pose less risk of gastric ulceration. Other side effects of NSAIDs that result from blockade of the synthesis of endogenous prostaglandins and thromboxane A₂ include disturbances in platelet function, the prolongation of gestation or spontaneous labor, premature closure of the patent ductus, and changes in renal function. Other risks include anaphylactoid reactions, angiodema, anemia, fluid retention, borderline elevations of one or more liver tests, and notable elevations of ALT or AST.

Morphine sulfate has been used in clinical studies to treat patients with chronic back pain. A once-daily pain product of morphine sulfate extended-release capsules identified as AVINZA® is stated by Ligand Pharmaceuticals Incorporated (San Diego, Calif., USA) to provide stable analgesia for one year in patients with chronic back pain, without increases in dose or the use of rescue medicines. Patients with chronic, moderate-to-severe back pain who took AVINZA® once-daily were reported to experience, on average, stable pain control for the duration of the study, as measured both by the lack of change in pain intensity, and by the stable average dose. In addition, AVINZA® was reported to maintain pain control while patients use approximately one less dose of rescue medicine per day compared to baseline. It was further reported that, with the exception of the improvement observed in the first month, there were no statistically significant or clinically meaningful changes in pain intensity during the one-year study, indicating that AVINZA® provided stable, long-term analgesia. Side effects were similar to those typically observed with opioid therapy, and included nausea, constipation and flu-like syndrome.

Crain and Shen in U.S. Pat. Nos. 5,472,943; 5,512,578 reissued as RE 36,457; 5,580,876; 5,767,125; 6,096,756; and 6,362,194 as well as U.S. Patent Application Publication No. 20020094947 A1 (the disclosures of which are incorporated herein by reference) describe methods and compositions of opioids for selectively enhancing the analgesic potency of a bimodally-acting opioid agonist and simultaneously attenuating anti-analgesia, hyperalgesia, hyperexcitability, physical dependence and/or tolerance effects associated with the administration of the bimodally-acting opioid agonist, by administering to a subject an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist. Also disclosed are methods and compositions of opioids for treating pain in a subject by administering to the subject an analgesic or sub-analgesic amount of a bimodally-acting opioid agonist and an amount of an excitatory opioid receptor antagonist effective to enhance the analgesic potency of the bimodally-acting opioid agonist and simultaneously attenuate anti-analgesia, hyperalgesia, hyperexcitability, physical dependence and/or tolerance effects of the bimodally-acting opioid agonist.

U.S. Patent Application Publication Nos. 20010006967 A1 and 20020094947 A1 (the disclosures of which are incorporated herein by reference) describe a method for selectively enhancing the analgesic potency of a bimodally-acting opioid agonist such as tramadol and simultaneously attenuating anti-analgesia, hyperalgesia, hyperexcitability, physical dependence and/or tolerance effects associated with the administration of the bimodally-acting opioid agonist. Disclosed are methods and compositions of tramadol in analgesic or sub-analgesic amounts and an opioid antagonist such as naltrexone or nalmefene.

U.S. Patent Application Publication No. 20010018413 A1 and U.S. Pat. No. 6,737,400 (published as U.S. Patent Application No. 20020173466 A1) (the disclosures of which are incorporated herein by reference) describe a method for treating a subject with irritable bowel syndrome (“IBS”) with an opioid antagonist. Disclosed are materials and methods for long-term administration of an opioid receptor antagonist at an appropriately low dose which will selectively antagonize excitatory opioid receptor functions, but not inhibitory opioid receptor functions, in myenteric neurons in the intestinal tract as well as in neurons of the central nervous system (“CNS”). The administration of the opioid receptor antagonist at a low dose reduces abdominal pain and stool frequency. Also disclosed are compositions for treating a subject with IBS, which comprise an effective dose of an opioid receptor antagonist, and a pharmaceutically acceptable carrier.

U.S. Patent Application Publication No. 2002013776 A1 (the disclosure of which is incorporated herein by reference) describes a method for increasing analgesic potency of a bimodally-acting opioid agonist in a subject, by inhibiting GM1-ganglioside in nociceptive neurons. The publication describes methods for treating pain, including methods for treating chronic pain, in a subject in need of treatment thereof. Additionally, a method is described for treating tolerance to or an addiction to a bimodally-acting opioid agonist in a subject in need of treatment thereof. A pharmaceutical composition of analgesic agents and a pharmaceutically-acceptable carrier is described.

International Publication No. WO 01/085150 (International PCT/US01/14644) (the disclosure of which is incorporated herein by reference) describes novel compositions and methods for enhancing potency or reducing adverse side effects of opioid agonists in humans, including with an opioid agonist and an opioid antagonist to differentially dose a human subject so as to either enhance analgesic potency without attenuating an adverse side effect of the agonist, or alternatively maintain the analgesic potency of the agonist while attenuating an adverse side effect of the agonist. Also described are novel opioid compositions and methods for the gender-based dosing of men and women.

U.S. Patent Application Publication No. 20030191147 A1 (the disclosure of which is incorporated herein by reference) describes novel dosage forms, pharmaceutical compositions, kits, and methods of administration of an opioid antagonist, including in an amount of at least about 0.0001 mg to about or less than about 1.0 mg, including from about 0.0001 mg to less than about 0.5 mg. Disclosed are solid oral dosage forms comprising an opioid antagonist and another active ingredient, such as an opioid agonist. Also disclosed are immediate release oral dosage forms and concurrent release dosage forms comprising an opioid antagonist and another active ingredient.

Although a variety of therapeutic agents have been used for treating pain and/or inflammation, the treatment of chronic pain or inflammation from a chronic condition is often still ineffective. In particular, chronic pain and/or chronic inflammation is often poorly managed or controlled even by the chronic administration of such agents. This may be due to the loss of potency of the agent and/or the development of side effects associated with chronic treatment with the agent.

### SUMMARY OF THE INVENTION

The present invention provides methods and materials, including novel compositions, dosage forms and methods of administration, useful for the treatment of arthritic conditions, inflammation associated with a chronic condition or chronic pain, including pain from arthritic conditions or inflammation using opioid antagonists, including combinations of opioid antagonists and opioid agonists. Methods and materials of the invention provide treatment for pain, wherein the pain is moderate to severe. Methods and materials of the present invention provide human subjects with alleviation of one or more symptoms or signs of the arthritic condition, inflammation associated with a chronic condition or chronic pain, including, for example, alleviation of pain, alleviation of stiffness and/or improvement of physical function. Methods and materials of the invention comprising opioid antagonists or combinations of opioid antagonists and agonists may optionally include one or more additional therapeutic agents.

In one aspect, the present invention is directed to methods and materials for treating an arthritic condition in a human subject by administering to the subject an opioid antagonist, wherein the amount of the antagonist is effective for enhancing the potency of an opioid agonist for alleviating one or more symptoms or signs associated with the arthritic condition.

In another aspect, the present invention is directed to methods and materials for treating an arthritic condition in a human subject by administering to the subject an opioid agonist and an opioid antagonist, wherein the amount of the agonist and the amount of the antagonist together are effective for alleviating one or more symptoms or signs associated with the arthritic condition.

In another aspect, the present invention is directed to methods and materials for inhibiting progression of an arthritic condition in a human subject by administering to the subject an opioid antagonist, wherein the amount of the antagonist is effective for enhancing the potency of an opioid agonist for inhibiting progression of the arthritic condition.

In another aspect, the present invention is directed to methods and materials for inhibiting progression of an arthritic condition in a human subject by administering to the subject an opioid agonist and an opioid antagonist wherein the amount of the agonist and the amount of the antagonist together are effective for inhibiting progression of the arthritic condition.

In another aspect, the present invention is directed to methods and materials for reversing damage associated with an arthritic condition in a human subject by administering to the subject an opioid antagonist, wherein the amount of the antagonist is effective for enhancing the potency of an opioid agonist for reversing damage associated with the arthritic condition.

In another aspect, the present invention is directed to methods and materials for reversing damage associated with an arthritic condition in a human subject by administering to the subject an opioid agonist and an opioid antagonist, wherein the amount of the agonist and the amount of the antagonist together are effective for reversing damage due to the arthritic condition.

In another aspect, the present invention is directed to methods and materials for treating inflammation associated with a chronic condition in a human subject by administering to the subject an opioid antagonist, wherein the amount of the antagonist is effective for enhancing the potency of an opioid agonist for alleviating one or more symptoms or signs associated with the chronic condition.

In another aspect, the present invention is directed to methods and materials for treating inflammation associated with inflammation in a human subject by administering to the subject an opioid agonist and an opioid antagonist, wherein the amount of the agonist and the amount of the antagonist together are effective for alleviating one or more signs or symptoms associated with the inflammation.

In another aspect, the present invention is directed to methods and materials for inhibiting tissue or cellular damage resulting from inflammation associated with a chronic condition in a human subject by administering to the subject an opioid antagonist, wherein the amount of the antagonist is effective for enhancing the potency of an opioid agonist for inhibiting the tissue or cellular damage resulting from the inflammation.

In another aspect, the present invention is directed to methods and materials of inhibiting tissue or cellular damage resulting from inflammation associated with a chronic condition in a human subject by administering to the subject an opioid agonist and an opioid antagonist, wherein the amount of the agonist and the amount of the antagonist together are effective for inhibiting the tissue or cellular damage resulting from the inflammation.

In another aspect, the present invention is directed to methods and materials for reversing tissue or cellular damage resulting from inflammation associated with a chronic condition in a human subject by administering to the subject an opioid antagonist, wherein the amount of the antagonist is effective for enhancing the potency of an opioid agonist for reversing the damage resulting from the inflammation.

In another aspect, the present invention is directed to methods and materials for reversing tissue or cellular damage resulting from inflammation associated with a chronic condition in a human subject by administering to the subject an opioid agonist and an opioid antagonist, wherein the amount of the agonist and the amount of the antagonist together are effective for reversing the damage due to the inflammation.

In another aspect, the present invention is directed to methods and materials for treating chronic pain by administering to a human subject with chronic pain an opioid antagonist, wherein the amount of the opioid antagonist is effective for enhancing the potency of an opioid agonist to attenuate the chronic pain. Chronic pain may result from various abnormal or compromised states (e.g., diseased), including but not limited to osteoarthritis, rheumatoid arthritis, psoriatic arthritis, back pain, cancer, injury or trauma.

In another aspect, the present invention is directed to methods and materials for treating chronic pain by administering to a human subject with chronic pain an opioid agonist and an opioid antagonist, wherein the amount of the agonist and the amount of the antagonist together are effective to attenuate the chronic pain.

In yet another aspect, the present invention is directed to methods and materials for dosing an opioid antagonist administered to a human subject. An amount of an opioid antagonist and an amount of an opioid agonist are administered to the subject. One or more symptoms or signs of an arthritic condition, inflammation associated with a chronic condition, or chronic pain are assessed. A level of the opioid antagonist or a surrogate of the opioid antagonist in a sample from the subject is measured. The amount of the opioid antagonist or the amount of the opioid agonist to the subject is adjusted based on the measured level.

In another aspect, the present invention is directed to methods and materials for dosing an opioid antagonist administered to a human subject. An amount of an opioid antagonist and an amount of an opioid agonist are administered to the subject. One or more symptoms or signs of an arthritic condition, inflammation associated with a chronic condition, or chronic pain are assessed. The amount of the opioid antagonist administered to the subject is adjusted if one or more of the assessed symptoms or signs are not alleviated to a desired extent.

In another aspect, the present invention is directed to methods and materials for dosing an opioid antagonist administered to a human subject. An amount of an opioid antagonist and an amount of an opioid agonist to the subject. A level of the opioid antagonist or a surrogate of the opioid antagonist in a sample from a subject is measured. The amount of the opioid antagonist administered to the subject is adjusted if the measured level is outside a predetermined range.

In another aspect, the present invention is directed to methods and materials for determining the amount of an opioid antagonist or opioid agonist to be administered to a human subject. One or more symptoms or signs of an arthritic condition, inflammation associated with a chronic condition, or chronic pain, in a human subject being administered an opioid antagonist and an opioid agonist is assessed. A level of the opioid antagonist or a surrogate of the opioid antagonist in a sample obtained from the human subject is measured. For example, the level of 6β-naltrexol can be measured as a surrogate. The 6β-naltrexol level (e.g., the concentration of 6β-naltrexol in a plasma sample) can be a surrogate marker for assessing one or more symptoms or signs of an arthritic condition, inflammation associated with a chronic condition, or chronic pain. On the basis of the measured level, the amount of the opioid antagonist or the amount of the opioid agonist for administration to the human subject is adjusted.

In another aspect, the present invention is directed to methods and materials for reducing the level of a biomarker in a human subject having an arthritic condition, inflammation associated with a chronic condition, or chronic pain, wherein a composition comprising an opioid antagonist and optionally an opioid agonist is administered to the human subject.

In yet another aspect, the present invention is directed to methods and materials for monitoring the response of a human subject being treated for an arthritic condition, inflammation associated with a chronic condition, or chronic pain, by administering an opioid antagonist and optionally an opioid agonist. The level of one or more one biomarker(s) in a first sample from the subject is determined prior to treatment with the opioid antagonist and optionally the opioid agonist. The level of the biomarker in at least a second sample from the subject is determined subsequent to the initial treatment with the opioid antagonist and optionally the opioid agonist. The level of the biomarker in the second sample is compared with the level of the biomarker in the first sample. A change in the level of the biomarker in the second sample compared to the level of the biomarker in the first sample indicates the effectiveness of the treatment.

One or more symptoms and signs of arthritic conditions, inflammation associated chronic conditions or chronic pain are alleviated (e.g., ameliorated, attenuated, reduced, diminished, blocked, inhibited or prevented), by methods and materials of the invention, for example, as measured by an alleviation (e.g., amelioration, attenuation, reduction, diminishment, blockage, inhibition or prevention) of pain, stiffness, or difficulty in physical function.

The present invention is directed to novel compositions, dosage forms, kits, and other materials comprising an opioid antagonist for use in or with the foregoing methods including wherein the amount of the antagonist is effective for enhancing the potency of an opioid agonist for alleviating one or more symptoms or signs associated with an arthritic condition, inflammation associated with a chronic condition, or chronic pain, and including compositions, dosage forms, kits, and other materials with an opioid agonist and an opioid antagonist, including wherein the amount of the agonist and the amount of the antagonist together are effective for alleviating one or more symptoms or signs associated with an arthritic condition, inflammation associated with a chronic condition, or chronic pain.

Symptoms and signs of arthritic conditions and inflammation resulting from chronic conditions, are alleviated (e.g., ameliorated, attenuated, reduced, diminished, blocked, inhibited or prevented), by methods and materials of the invention, for example, as measured by an alleviation (e.g., amelioration, attenuation, reduction, diminishment, blockage, inhibition or prevention) of pain, stiffness, and/or difficulty in physical function.

Thus, the present invention provides methods and materials comprising opioid antagonists, including opioid agonists and antagonists, that provide greater pain relief, better pain control, improved function, with no change in side effect profile, even with chronic administration including as compared with methods and materials without opioid antagonists. Advantages of methods and materials of the invention include enhanced and prolonged analgesia, prevention of tolerance and continued protection against tolerance even with chronic administration, reversal of opioid agonists-induced hyperalgesia, prevention of physical dependence or withdrawal, decreased rewarding/euphoric side effect, and/or decreased potential for relapse/addiction.

### BRIEF DESCRIPTION OF THE FIGURES

FIG. 1 shows plasma concentrations (mean±SEM) of oxycodone (ng/mL) in the three treatment groups from the clinical study conducted as described in Example 1: oxycodone QID represented as red circles (o); the combination drug of oxycodone and naltrexone QID represented as green triangles (Δ); and the combination drug of oxycodone and naltrexone BID represented as pink squares (□).

FIG. 2 shows plasma concentrations (mean±SEM) of oxymorphone (ng/mL) in the three treatment groups from the clinical study conducted as described in Example 1: oxycodone QID represented as the bar having diagonal lines; the combination drug of oxycodone and naltrexone QID represented as the bar having diamonds; the combination drug of oxycodone and naltrexone BID represented as the darker bar having polka dots.

FIG. 3 shows plasma concentrations (median±quartiles) of oxycodone (ng/mL) after the final dose in the three treatment groups from the clinical study conducted as described in Example 1.

FIG. 4 shows log-transformed plasma concentrations (median±quartiles) of oxycodone after the final dose in the three treatment groups from the clinical study conducted as described in Example 1.

FIG. 5 shows log-transformed plasma concentrations (median±quartiles) of oxymorphone after the final dose in the three treatment groups from the clinical study conducted as described in Example 1.

FIG. 6 shows dose-normalized plasma concentrations (mean±SEM) of oxycodone (ng/mL) in the three treatment groups from the clinical study conducted as described in Example 1: oxycodone QID represented as red circles (o); the combination drug of oxycodone and naltrexone QID represented as green triangles (Δ); and the combination drug of oxycodone and naltrexone BID represented as pink squares (□).

FIG. 7 shows plasma concentrations (mean±SEM) of 6β-naltrexol (pg/mL) for two of the treatment groups from the clinical study conducted as described in Example 1: the combination drug of oxycodone and naltrexone QID represented as the bar having diamonds; the combination drug of oxycodone and naltrexone BID represented as the darker bar having dots.

FIG. 8 shows efficacy measures versus oxycodone concentrations after the final dose for the three treatment from the clinical study conducted as described in Example 1: oxycodone QID represented as black circles; the combination drug of oxycodone and naltrexone BID represented as red squares; the combination drug of oxycodone and naltrexone QID represented as green diamonds.

FIG. 9 shows efficacy measures versus oxymorphone concentrations after the final dose for the three treatment groups from the clinical study conducted as described in Example 1: oxycodone QID represented as black circles; the combination drug of oxycodone and naltrexone BID represented as red squares; the combination drug of oxycodone and naltrexone QID represented as green diamonds.

FIG. 10 shows efficacy measures versus 6β-naltrexol concentrations after the final dose for two of the treatment groups from the clinical study conducted as described in Example 1: the combination drug of oxycodone and naltrexone BID represented as black circles; the combination drug of oxycodone and naltrexone QID represented as red squares.

FIG. 11 shows the percent change in pain intensity reported by some of the subjects in Table 23 vs. 6β-naltrexol plasma concentrations measured for those subjects, as described in Example 3.

FIG. 12 shows the percent change in pain intensity reported by subjects in Table 23 who received the BID dosing regimen vs. 6β-naltrexol plasma concentrations measured for those subjects as described in Example 3.

FIG. 13 shows steps in a process for the preparation of dosage forms of opioid agonist and opioid antagonist.

### DETAILED DESCRIPTION OF THE INVENTION

The present invention provides methods and materials, including novel compositions, dosage forms and methods of administration, useful for the treatment of arthritic conditions, inflammation associated with a chronic condition, and/or chronic pain, including pain or other symptoms or signs associated with arthritic conditions or inflammation associated with chronic conditions, using opioid antagonists, including combinations of opioid antagonists and opioid agonists. The methods and materials provide human subjects with alleviation of one or more of such symptoms or signs including, for example, reduced pain, reduced stiffness and/or improved physical function. Methods and materials of the invention comprising opioid antagonists, including combinations opioid antagonists and agonists may optionally include one or more additional therapeutic agents.

The present invention provides methods and materials for treating arthritic conditions and/or inflammation associated with chronic conditions in a human subject by administering to the subject an opioid antagonist or an opioid agonist with an opioid antagonist. For example, the amount of an opioid antagonist is effective to enhance the potency of an opioid agonist for alleviating one or more symptoms or signs associated with an arthritic condition or inflammation associated with a chronic condition, for example, symptoms or signs such as pain, stiffness or difficulty in physical function.

The present invention provides methods and materials for inhibiting progression of an arthritic condition or inflammation associated with chronic conditions in a human subject by administering to the subject an opioid antagonist or an opioid agonist with an opioid antagonist. For example, the amount of an opioid antagonist is an amount effective for enhancing the potency of an opioid agonist for inhibiting progression of the arthritic condition or chronic conditions associated with inflammation. The present invention thus provides methods and materials for inhibiting the change or progression in a subject from a normal or uncompromised state (e.g., healthy) to an abnormal or compromised state (e.g., diseased), as indicated, for example, by a symptom or sign associated with an arthritic condition, inflammation from a chronic condition or chronic pain. The progression of an arthritic condition or inflammation associated with a chronic condition can be measured by a variety of methods, including by radiography, by measuring levels of cytokines and/or by measuring B cell and T cell subtype ratios.

The present invention provides methods and materials for reversing damage associated with an arthritic condition or inflammation associated with chronic conditions in a human subject comprising administering to the subject an opioid antagonist or an opioid agonist with an opioid antagonist. For example, the amount of an opioid antagonist is an amount effective for enhancing the potency of an opioid agonist for reversing damage due to the arthritic condition or inflammation associated with chronic conditions. The present invention thus provides methods and materials for reversing the change or progression in a subject from a normal or uncompromised state to an abnormal or compromised state as indicated, for example, by a symptom or sign associated with an arthritic condition, inflammation from a chronic condition or chronic pain. The progression of the arthritic condition or inflammation associated with chronic conditions can be measured by a variety of methods, including by radiography, by measuring levels of cytokines and/or by measuring B cell and T cell subtype ratios.

The present invention provides methods and materials for treating chronic pain by administering to a human subject with chronic pain an opioid antagonist or the amount of an opioid agonist with an opioid antagonist. Chronic pain can include pain that is headache, lower back pain, cancer pain, arthritis pain, infection pain, neurogenic pain or psychogenic pain. Methods and materials are effective for the treatment of moderate to severe pain and particularly severe pain. For example, the amount of an opioid antagonist is an amount effective for enhancing the potency of an opioid agonist for alleviating the chronic pain. The pain intensity of the chronic pain is thereby alleviated (e.g., ameliorated, attenuated, reduced, diminished, blocked, inhibited or prevented).

In the treatment of chronic pain, of inflammation associated with a chronic condition or of an arthritic condition, an opioid antagonist or the combination of an opioid agonist and an opioid antagonist can each be administered at least once daily for at least one week, alternatively at least once daily for at least two weeks, at least once daily for at least three weeks, or at least once daily for a longer time. The method for treating chronic pain, treating inflammation associated with a chronic condition, or treating an arthritic condition may comprise administering the opioid antagonist or each of the opioid agonist and the opioid antagonist no more than twice daily for at least one week, alternatively no more than twice daily for at least two weeks, alternatively no more than twice daily for at least three weeks, or no more than twice daily for a longer time. The method for treating chronic pain, treating inflammation associated with a chronic condition, or treating an arthritic condition may comprise administering to the subject a daily amount of the opioid antagonist that is less than 0.004 mg, alternatively 0.002 mg or less.

The present invention provides compositions that comprise an opioid antagonist (e.g., an excitatory opioid receptor antagonist). Such compositions additionally preferentially comprise an opioid agonist (e.g., a bimodally-acting opioid agonist), and optionally a pharmaceutically acceptable carrier or excipient for administration to a subject, preferably a human, in need thereof. Such compositions optionally comprise an additional therapeutic agent.

It is contemplated that the present methods and compositions may be employed for the treatment of inflammation associated with chronic conditions (including inhibiting progression of and/or reversing damage associated with inflammation), including the chronic conditions associated with inflammation in and around joints, muscles, bursae, tendons vertebrae, or fibrous tissue. Such methods and compositions provide reduced pain, reduced stiffness and/or improved physical function.

It is also contemplated that the present methods and compositions may be employed for the treatment of chronic conditions (including inhibiting progression of and/or reversing damage associated with chronic conditions). Chronic conditions include, for example, arthritic conditions such as osteoarthritis, rheumatoid arthritis, and psoriatic arthritis. For example, the present methods and compositions may be used to treat one or more symptoms or signs of osteoarthritis of the joint, (such as a hip or knee) or the back (for example, the lower back). Chronic conditions also include, for example, conditions associated with or resulting from pain such as chronic pain, including pain associated with or arising from cancer, from infection or from the nervous system (e.g., neurogenic pain such as peripheral neurogenic pain following pressure upon or stretching of a peripheral nerve or root or having its origin in stroke, multiple sclerosis or trauma, including of the spinal cord). Chronic conditions also include, for example, conditions associated with or arising from psychogenic pain (e.g., pain not due to past disease or injury or visible sign of damage inside or outside the nervous system).

The present methods and compositions may also be employed for the treatment of other arthritic conditions, including gout and spondylarthropathris (including ankylosing spondylitis, Reiter's syndrome, psoriatic arthropathy, enterapathric spondylitis, juvenile arthropathy or juvenile ankylosing spondylitis, and reactive arthropathy). The present methods and compositions may be used for the treatment of infectious or post-infectious arthritis (including gonoccocal arthritis, tuberculous arthritis, viral arthritis, fungal arthritis, syphlitic arthritis, and Lyme disease).

Additionally, the present methods and compositions may be used for the treatment of arthritis associated with various syndromes, diseases, and conditions, such as arthritis associated with vasculitic syndrome, arthritis associated with polyarteritis nodosa, arthritis associated with hypersensitivity vasculitis, arthritis associated with Luegenec's granulomatosis, arthritis associated with polymyalgin rheumatica, and arthritis associated with joint cell arteritis. Other preferred indications contemplated for employing the compositions and methods herein include calcium crystal deposition arthropathies (such as pseudo gout), non-articular rheumatism (such as bursitis, tenosynomitis, epicondylitis, carpal tunnel syndrome, and repetitive use injuries), neuropathic joint disease, hemarthrosis, Henoch-Schonlein Purpura, hypertrophic osteoarthropathy, and multicentric reticulohistiocytosis. Other preferred indications contemplated for employing the compositions and methods herein include arthritic conditions associated with surcoilosis, hemochromatosis, sickle cell disease and other hemoglobinopathries, hyperlipo proteineimia, hypogammaglobulinemia, hyperparathyroidism, acromegaly, familial Mediterranean fever, Behat's Disease, lupus (including systemic lupus erythrematosis), hemophilia, and relapsing polychondritis.

The methods and compositions for treating arthritic conditions, inflammation associated with chronic conditions or chronic pain alleviate (e.g., ameliorate, attenuate, reduce, diminish, block, inhibit or prevent) at least one symptom or sign of an arthritic condition, inflammation associated with a chronic condition, or chronic pain. For example, the methods and compositions may alleviate one or more of pain intensity, stiffness, or difficulty in physical functions. The methods and compositions may attenuate one or more symptoms or signs of an arthritic condition, inflammation associated with a chronic condition, or chronic pain, wherein the sign or symptom after administration of the composition is ameliorated as compared to the sign or symptom before administration of the composition.

The present invention is directed to compositions, dosage forms, and kits with an opioid antagonist, including an opioid antagonist in combination with an opioid agonist, wherein the amount of the antagonist enhances the potency of an opioid agonist or wherein the amounts of the agonist and the amount of the antagonist together are effective to alleviate (e.g,, ameliorate, attenuate, reduce, diminish, block, inhibit or prevent) one or more symptoms or signs of an arthritic condition, inflammation associated with a chronic condition, or chronic pain. The invention further relates to methods for administering to human subjects such compositions, dosage forms, and kits. Optionally, the present methods and materials may further comprise administering a pharmaceutically acceptable carrier or excipient for administration to the subject, preferably a human, in need thereof. Further, optimally, the present methods and materials may comprise an additional therapeutic agent.

The present invention also provides methods for treating a subject with pain from an arthritic condition or inflammation associated with a chronic condition, comprising administering an amount of opioid antagonist effective to enhance the pain-alleviating potency of an opioid agonist, including an endogenous opioid agonist and optionally a pharmaceutically acceptable carrier or excipient for administration to the subject, preferably a human, in need thereof, whereby the pain is alleviated. Such methods optionally include additionally administering an opioid agonist, and in such methods, the amount of antagonist is effective to enhance the pain-alleviating potency of the administered agonist.

The present invention also provides methods and materials for treating an arthritic condition or inflammation associated with chronic conditions. The methods comprise administering to a human subject an amount of an opioid antagonist or the combination of an opioid agonist and an opioid antagonist that is effective to enhance potency of the agonist and/or to alleviate one or more symptoms or signs of an arthritic condition or inflammation associated with a chronic condition, including for example, as measured by a suitable index, scale or measure. The attenuation of one or more symptoms or signs of an arthritic condition or of inflammation associated with a chronic condition may be measured on the WOMAC Osteoarthritis Index or one of its subscales (in other words, the pain, stiffness, or physical function subscales of the WOMAC Osteoarthritis Index). Any suitable version of the WOMAC OA Index may be used, including, for example, Version 3.0 or Version 3.1. Any suitable scale may be used as well. The WOMAC OA Index is available in Likert and Visual Analog scaled formats, either of which may be employed in the present methods. WOMAC values can be considered as surrogate markers for the diagnosis, prognosis, monitoring or treatment of an arthritic condition, inflammation from a chronic condition, and/or chronic pain. The WOMAC values represent a subjective surrogate marker. Alternatively or additionally, the attenuation of one or more symptoms or signs may be measured on another suitable index, scale or measure, such the Australian/Canadian (AUSCAN) Osteoarthritis Hand Index or the Osteoarthritis Global Index (OGI). The AUSCAN 3.0 Index and User Guide are currently available from http://www.womac.org/contact/index.cfm, as are the WOMAC 3.1 Osteoarthritis Index and User Guide. Another suitable measure of attenuation is the Definition of Improvement in Rheumatoid Arthritis described in Felson et al., Arthritis & Rheumatism 38:727-735 (1995) incorporated herein by reference. This measure, which also may be designated as the ACR (American College of Rheumatology) 20 improvement, is a composite defined as both improvement of 20% in the number of tender and number of swollen joints, and a 20% improvement in three of the following five: patient global, physician global, patient pain, patient function assessment, and C-reactive protein (CRP). Another suitable measure is described by Paulus et al., Arthritis & Rheumatism 33:477-484 (1990) incorporated herein by reference. Paulus et al. provides a definition of improvement based on a set of measures that discriminate between active second-line drug treatment and placebo. These include a 20% improvement in morning stiffness, erythrocyte sedimentation rate (ESR), joint tenderness score, and joint swelling score and improvement by at least 2 grades on a 5-grade scale (or from grade 2 to grade 1) for patient and physician global assessments of current disease severity. Current disease severity can be measured in a variety of ways, including patient or physician global assessments, patient or physician assessments of joint tenderness, joint swelling stiffness, pain, or physical function, cytokine levels, B-cell or T-cell subtype ratios, erythrocyte sedimentation rate (ESR), or C-reactive protein. Suitable measures of attenuation of one or more symptoms or signs, of inhibiting the progression of an arthritic condition or chronic condition, or of reversing tissue or cellular damage include measuring current disease severity. Other indexes, definitions, measures, or scales may also be used for measuring attenuation of one or more symptoms or signs, inhibition of progression, or reversal of tissue or cellular damage.

The present invention provides methods and materials for alleviating pain associated with arthritic conditions or inflammation associated with chronic conditions. For example, the amount of an opioid antagonist or the combination of an opioid agonist and an opioid antagonist may be effective to enhance the potency of the agonist and/or to attenuate (e.g., ameliorate, alleviate, reduce, diminish, block, inhibit or prevent) (1) the pain felt by the subject when walking on a flat surface; (2) the pain felt by the subject when going up or down stairs; (3) the pain felt by the subject at night while in bed; (4) the pain felt by the subject that disturbs the sleep of the subject; (5) the pain felt by the subject while sitting or lying down; and/or (6) the pain felt by the subject while standing.

Alternatively or additionally, the present invention provides methods and materials for alleviating stiffness associated with arthritic conditions or inflammation associated with chronic conditions. For example, the amount of an opioid antagonist or the combination of an opioid agonist and an opioid antagonist may be effective to enhance the potency of the agonist and/or to attenuate (e.g., ameliorate, alleviate, reduce, diminish, block, inhibit or prevent) (1) the severity of the stiffness felt by the patient after the subject first woke up in the morning; (2) the severity of the stiffness felt by the subject after sitting or lying down later in the day; and/or (3) the severity of the stiffness felt by the subject while resting later in the day.

Alternatively or additionally, the present invention provides methods and materials for alleviating difficulty in physical function associated with arthritic conditions or inflammation associated with chronic conditions. For example, the amount of an opioid antagonist or the combination of an opioid agonist and an opioid antagonist may be effective to enhance the potency of the agonist and/or to attenuate (e.g., ameliorate, alleviate, reduce, diminish, block, inhibit or prevent) (1) the difficulty had by the subject when going down stairs; (2) the difficulty had by the human subject when going up stairs; (3) the difficulty had by the subject when getting up from a sitting position; (4) the difficulty had by the subject while standing; (5) the difficulty had by the subject when bending to the floor; (6) the difficulty had by the patient when walking on a flat surface; (7) the difficulty had by the human subject when getting in or out of a car or bus; (8) the difficulty had by the subject while going shopping; (9) the difficulty had by the patient when getting out of bed; (10) the difficulty had by the subject when putting on socks, or panty hose or stockings; (11) the difficulty had by the subject while lying in bed; (12) the difficulty had by the subject when getting in or out of the bathtub; (13) the difficulty had by the subject while sitting; (14) the difficulty had by the patient when getting on or off the toilet; (15) the difficulty had by the subject while doing heavy household chores; and/or (16) the difficulty had by the subject while doing light household chores.

Biomarkers have been identified, as described herein, that are useful in methods and materials for the treatment of an arthritic condition, inflammation from a chronic condition and/or chronic pain, including pain from an arthritic condition or inflammation. A biomarker is a molecular entity, for example, a biochemical in the body, which has a molecular feature that makes it useful for diagnosis, prognosis, monitoring or treatment of a subject, including, for example, measuring progress of disease or effects of treatment. Biomarkers can include inflammatory biomarkers. An inflammatory biomarker can be any suitable biomarker known or recognized as being related to an inflammatory condition, including but not limited to: pro-inflammatory or anti-inflammatory, such as cytokines, interleukin-1 through 17, including interleukin-1α (IL1a), interleukin-1β (IL1b), IL2, IL4, IL5, IL6, IL8, IL10, IL13, tumor necrosis factor alpha (TNFα), GM-CSF, interferon gamma (IFN-γ); markers of systemic inflammation, including, for example, CRP; certain cellular adhesion molecules such as e-selectin, integrins, ICAM-1, ICAM-3, BL-CAM, LFA-2, VCAM-1, NCAM, PECAM, and neopterin; and B61; leukotriene, thromboxane, isoprostane, serum amyloid A protein, fibrinectin, fibrinogen, leptin, prostaglandin E2, serum procalcitonin, soluble TNF receptor 2 (sTNFr2), erythrocyte sedimentation rate, erythema; elevated white blood count (WBC), including percent and total granulocytes (polymorphonuclear leukocytes) monocytes, lymphocytes and eosinophils; and increased erythrocyte sedimentation rate. Further biomarkers of an inflammatory condition may include decreased levels of pre-albumin and albumin.

A sample that contains or may contain a biomarker can be obtained, including a biological sample. Biological sample refers to a sample obtained from an organism (e.g., a human subject) or from components (e.g., cells, tissues or fluids) of an organism. The sample can be a body fluid, tissue, or cell, including, but not limited to, blood, plasma, serum, blood cells (e.g., white cells), tissue or biopsy samples (e.g., tumor biopsy), urine, saliva, tears, sputum, synovial fluid, cerebrospinal fluid, peritoneal fluid, and pleural fluid, or cells therefrom. An exemplary sample is a plasma sample. Biological samples can also include sections of fluids, tissues or cells such as frozen sections taken for histological purposes.

Samples can be analyzed for the presence of biomarkers by a variety of methods. Candidate biomarkers in such samples can include cytokines (e.g., objective biomarkers). Measurement of cytokines can be carried out in a number of ways known to those with skill in the art. Methods are available which can detect cytokines individually using traditional ELISA techniques (for example, Quantikine kits, available from R&D Systems, Minneapolis, Minn.), or several cytokines can be detected simultaneously, using liquid or solid based array systems. For example, Luminex (Austin, Tex.) has developed a liquid array system based on microspheres, wherein the spheres contain a mixture of two fluorophors. The ratio of the two dyes within the mix is precisely controlled, and gives a unique spectral signature to 100 different species of the microbeads. Each of these 100 different species is then coated with known and unique capture reagents, capable of interacting with molecules of interest within a complex mixture such as serum, plasma or cell culture supernatant. These binder molecules can be entities such as antibodies, oligonucleotides, peptides and receptors. A reporter molecule, specific for the analyte molecule of interest, is then used to quantitate binding. The Luminex system requires a specific detector that uses microfluidics to detect individually labeled beads.

Various kits are available for use with this Luminex technology, including the Biosource International (Camarillo, Calif., www.biosource.com) human cytokine ten-plex antibody bead kit. This kit measures members of two classes of cytokines, the TH1/TH2 and the inflammatory cytokines. The TH1/TH2 set includes IL-2, -4, -5, -10, INFγ while the inflammatory set is IL-1β, IL-6, IL-8, GM0CSF, and TNF α. Linco (St. Charles, Mo., www.lincoresearch.com) makes 13, 21, or 22-plex kits for cytokine measurement. The 22-plex kit can simultaneously measure IL-1α, IL-1β, IL-2, -4, -5, -6, -7, -8, -10, -12p70, -13, -15, -17, Eotaxin, G-CSF, GM-CSF, IFNγ, IP-10, MCP-1, MIP-1α, TNFα and RANTES. Another vendor, R & D Systems (Minneapolis, Minn., www.rndsystems.com) makes a kit for the detection of twelve cytokines, including INFγ, bFGF, GM-CSF, G-CSF, IL-2, -4, -5, -6, -8, -10, -17, IL-1β, IL-1α, IL-Ira, TNFα, VEGF, ENA-78, MIP-1, MCP-1, RANTES, and Tpo. Upstate (Charlottesville, Va., www.upstate.com) sells a variety of cytokine detection kits for use with the Luminex system that can detect up to 22 cytokines including IL-1α, IL-1β, IL-2, -3, -4, -5, -6, -7, -8, -10, -12(p40), -12(p70), -13, -15, IP-10, Eotaxin, IFNγ, GM-CSF, MCP-1, MIP-1α, RANTES, and TNFα. Qiagen (Valencia, Calif., www.qiagen.com) sells a kit capable of detecting 11 analytes at once, including Eotaxin, MCP-1, RANTES, GM-CSF, INFγ, IL-1α, IL-1, IL-2, -4, -5, -6, -8, 10, -12p70, and IL-13. Finally, BIORAD (Hercules, Calif., www.biorad.com) sells kits that can detect up to 17 cytokines at once, including: IL-1β, IL-2, -4, -5, -6, -7, -8, -10, -12p70, -13, -17, G-CSF, GM-CSF, INFγ, MCP-1, MIP-1, and TNFα. In addition, there are other vendors which have similar kits available for purchase for use with the Luminex system.

Other liquid array systems are available for detection of cytokines such as the CBA System developed by BD Bioscience/Pharmingen (Franklin Lakes, N.J., www.bdbiosciences.com). The CBA system also uses coated beads for detection of analytes. The beads are coated with binding molecules, and bound analyte is detected in a ‘sandwich’ assay using a phycoerytherin labeled antibody specific for that analyte in a standard flow cytometer. BD Bioscience/Pharmingen sells kits for detecting several (1-7) analytes at once and examples of these kits are the human TH1/TH2 kit that measures IL-2, -4, -6, 10, TNFα and INFγ, or the human inflammation kit which measures IL1β, IL6, IL8, IL 10, TNFα and IL 12p70. Bender MedSystems (Vienna, Austria, www.bendermedsystems.com) has developed a product line, the FlowCytomix system, for use with flow cytometer that consists of microbeads coated with antibodies which will interact with various cytokines. The beads are of varying sizes and have unique spectral qualities due to varying amounts of an internal fluorescent dye, and these properties allow the identification of each type of beads within a mixture of beads. Bender MedSystems's multicytokine kit measures several cytokines at once, and those to choose from include INFγ, IL1β, IL-2, -4, -5, -6, -8, -12, MCP-1, TNFα. Bender also sells a TH1/TH2 kit which measures human IL-1β, IL-2, -4, -5, -6, -8, -10, TNFα, TNFβ and INFγ simultaneously.

In addition to the fluid based systems discussed above, methodologies are available for measuring several cytokines at once in solid based array systems. For example, mini array ELISA systems have been used which measure seven different cytokines, TNF-α, IFNα, IFNγ, IL-1α, IL-1β, IL-6, and IL-10 (see Moody et al, BioTechniques 31:186-194 (July 2001)). Biochips have been developed for cytokine measurement (see Huang et al, CANCER RESEARCH 62, 2806-2812, May 15, 2002) wherein 43 cytokines can be detected including GM-CSF, G-CSF, IL-1α, IL-1β, IL-2, -3, -4, -5, 6, -8, -10, -12, -13, TNFα and VEGF. Array systems on glass slides have been developed (Tam et al. Journal of Immunological Methods 261: 157-165 (2002)), for example, capable of measuring eight cytokines including INFγ, IL-2, -4, -5, -6, -10 and -13 and TNFα), or rolling circle amplified-antibody arrays which can measure up to 75 cytokines simultaneously (Schweitzer et al, Nature Biotechnology 20: 359-365 (2002)) including IL-1α, IL-1β, IL-2, -4, -5, -6, -8, 10, -12, TNFα, RANTES and VEGF.

Other array systems, capable of acting either as fluid- or solid-based systems, are available from Pointilliste (Mountain View, Calif., http://www.pointilliste.com). This flexible technology is comprised of self assembling arrays in which the user is able to specifically select the analytes they wish to study. A reporter molecule, specific for the analyte molecule of interest, is then used to quantitate binding. As used herein, the measurements are done on a solid support where capture antibody arrays are applied to a ‘canvas’, wherein each canvas contains up to 96 arrays, and each array may contain up to 625 addressable spots. In this way, each canvas may contain up to 14 million unique, addressable molecules. Anti-cytokine arrays can be prepared in this system, making use of paired antibodies sets such as for example, Cytosets, available from BioSource International. A commercial human Th1/Th2 cytokine canvas is available from Pointilliste and was used as described in Example 4.

One or more cytokines can be employed as biomarkers for treatment using methods and materials as described herein. For example, one or more cytokines can be employed as a biomarker for treatment of an arthritic condition, inflammation associated with a chronic condition, and/or chronic pain, including pain from an arthritic condition or inflammation. One or more cytokines can be used as a biomarker of the existence or extent (e.g., diagnosis, prognosis, monitoring) of an arthritic condition, of inflammation associated with a chronic condition, and/or of chronic pain, including pain from arthritic conditions or inflammation. Alternatively or additionally, one or more cytokines can be used as a biomarker to assess the treatment of an arthritic condition, inflammation associated with a chronic condition, and/or chronic pain, including pain from an arthritic condition or inflammation. Examples of cytokines contemplated for such use as biomarkers include IL1α, IL1, IL2, IL4, IL5, IL6, IL10, IL13, GM-CSF, interferon-γ and TNFα. Preferably, the cytokines TNFα, IL6, IL4, and/or are used as biomarkers.

Cytokines can be measured as biomarkers before, during and/or after the administration of an opioid agonist, an opioid antagonist, or a combination of an opioid antagonist and opioid agonist. When cytokines are to be employed as biomarkers for a subject, one or more cytokine levels for that subject are measured. Cytokines can be employed as biomarkers, for example, for monitoring, diagnosing, prognosing and/or treating the subject, including but not limited to selecting dose amounts and/or dosing regimens of an opioid antagonist alone or in combination with an opioid agonist.

Level(s) of one or more cytokines, for example, plasma levels, can be measured in a subject at risk for, or seeking, for example, diagnosis, prognosis, monitoring and/or treatment of, or reporting, one or more signs or symptoms of, an arthritic condition, inflammation associated with a chronic condition, and/or chronic pain, including pain from an arthritic condition or inflammation. For example, depending on the measured cytokine level(s), an appropriate treatment can be selected and administered. The measured cytokine level(s) can be used to determine whether and how much opioid agonist and/or opioid antagonist are administered. Furthermore, for example, the dose amount and/or dosing regimen of an opioid agonist, an opioid antagonist, or a combination of an opioid antagonist and opioid agonist can be selected based upon the measured cytokine level(s). For example, if one or more of the measured cytokine levels is above a value, a physician can choose to treat a subject by administering an opioid agonist, an opioid antagonist, or a combination of opioid antagonist and opioid agonist. The value can be a predetermined value or a value determined at the time of or after measurement of the cytokine level(s). As another example, a physician can select a higher or lower amount of opioid agonist and/or a higher or lower amount of antagonist for administration. As yet another example, a more frequent or less frequent dosing regimen can be selected based on the measured cytokine level(s). For example, if the level of cytokines are higher than desired, an opioid antagonist can be dosed more frequently, or if the level of cytokines are lower than desired, an opioid antagonist can be dosed less frequently.

Level(s) of one or more cytokines, for example, plasma levels, can be measured for a subject who has already received or who is receiving treatment for an arthritic conditions, inflammation associated with a chronic condition, and/or chronic pain, including pain from an arthritic condition or inflammation. The measured cytokine level(s) can be used to determine whether appropriate amounts and regimens have been or are being employed for treating the subject. For example, level(s) of one or more cytokines can be measured in a subject receiving treatment for an arthritic condition, inflammation associated with a chronic condition, and/or chronic pain, including pain from an arthritic condition or inflammation. If the one or more of the measured cytokine levels is above a value, the treatment can be adjusted by administering a greater or lesser amount of an opioid agonist, an opioid antagonist, or a combination of opioid antagonist and opioid agonist and/or by altering the dosing regimen. The value can be a predetermined value or a value determined at the time of or after measurement of the cytokine level(s).

Concentrations of cytokines, for example, plasma concentrations, can be used as biomarkers in adjusting the administration of an opioid antagonist to a subject. A single cytokine concentration can be selected to evaluate whether a subject is in need of treatment. As an example, if a subject has a plasma concentration of TNFα which is higher than about 0.08 ng/ml, alternatively higher than 0.2 ng/ml, the subject is administered more opioid antagonist and/or more opioid agonist, by administering higher dose amounts and/or by administering on a more frequent dosing regimen. As another example, if the subject has a plasma concentration of TNFαwhich is about 0.08 ng/ml or lower, alternatively lower than 0.2 ng/ml, either the administration of opioid antagonist is not changed, or the subject is administered less opioid antagonist and/or less opioid agonist, by administering lower dose amounts and/or by administering on a less frequent dosing regimen. As another example, if a subject has a plasma concentration of IL4 which is higher than about 0.23 ng/ml, the subject is administered more opioid antagonist and/or more opioid agonist, by administering higher dose amounts and/or by administering on a more frequent dosing regimen. As another example, if the subject has a plasma concentration of IL4 which is about 0.23 ng/ml or lower, either the administration of opioid antagonist is not changed, or the subject is administered less opioid antagonist and/or less opioid agonist, by administering lower dose amounts and/or by administering on a less frequent dosing regimen. As another example, if a subject has a plasma concentration of IL6 which is higher than about 0.18 ng/ml, the subject is administered more opioid antagonist and/or more opioid agonist, by administering higher dose amounts and/or by administering on a more frequent dosing regimen. As another example, if the subject has a plasma concentration of IL6 which is about 0.18 ng/ml or lower, either the administration of opioid antagonist is not changed, or the subject is administered less opioid antagonist and/or less opioid agonist, by administering lower dose amounts and/or by administering on a less frequent dosing regimen.

One or more cytokine concentrations can be used as biomarkers in adjusting the administration of an opioid antagonist to a subject. For example, one or more of the concentrations of IL1α, IL1β, IL2, IL4, IL5, IL6, IL10, IL13, GM-CSF, interferon-γ and TNFα can be used to determine or adjust the treatment of an arthritic conditions, inflammation associated with a chronic condition, and/or chronic pain, including pain from an arthritic condition or inflammation.

The plasma concentration-effect relationship of low dose of an opioid antagonist when administered with an opioid agonist has been represented for the first time by the Emax composite model:
E[Emax1(Cpⁿ¹)/EC51ⁿ¹+Cpⁿ¹]+[Emax2(Cpⁿ²)/EC52ⁿ²+Cpⁿ²]
where the respective Emax values represent maximum effect for a given drug; EC51 and EC52 represent the potencies, for the drug notated as either 1 or 2, respectively (in other words, EC51 is not the concentration having 51% of the maximal effect, but rather EC51 is the concentration having a particular potency (e.g. 50% of the maximal effect for Effect No. 1); the respective values for C are the concentrations of drugs notated as 1 or 2, and the values of n₁ and n₂ that correspond to the sigmoidicity factors that are associated with particular EC values. In the Emax composite model, “+” is used to indicate absolute values; sometimes it is shown as a “−” which reflects a negative second term.

The Emax composite model is a recognized composite model for PK/PD data analysis set forth, for example, in Gabrielsson et al., PHARMACOKINETIC/PHARMACODYNAMIC DATA ANALYSIS: CONCEPTS AND APPLICATIONS, pp. 191-193 and 801-808 (2000), and the computer command files provided with the reference and described, including with examples of the computer printouts on pages 801-808, all of which is incorporated by reference herein. However, it is believed that the Emax composite model has not previously been utilized for the analysis of PK data from administering low doses of opioid antagonists such as naltrexone for enhancing the potency of opioid agonists such as oxycodone, as described herein. From the plasma concentration-effect data obtained and described in Example 3, it is contemplated that the opioid antagonist, at lower plasma concentrations, is impacting the total effect (percent change in pain intensity), primarily as described by the terms of the equation denoted with a 2.

The recognition of the applicability and utility of a composite model as shown above enables the selection of preferred and/or suitable ranges for the combined use of an opioid antagonist with an opioid agonist as described herein. The composite model provides the relative contribution of an opioid antagonist with respect to enhancing pain relief, for example, as measured by a reduction in pain intensity. The effective percentage decrease in pain intensity, E, has been found to be described by a relatively wide scope of preferred plasma concentrations by the Emax composite model, as described in Example 3 and as shown in the data and Figures described herein.

An effective amount to alleviate (e.g., ameliorate, attenuate, reduce, diminish, block, inhibit or prevent) symptom or sign of an arthritic condition or inflammation associated with chronic conditions refers to an amount of opioid antagonist or combination of opioid agonist and antagonist with or without one or more additional therapeutic agents which elicits alleviation (e.g., amelioration, attenuation, reduction, diminishment, blockage, inhibition or prevention) of at least one symptom or sign of an arthritic condition or inflammation associated with chronic conditions (e.g., pain) upon administration to a subject (e.g., patient) in need thereof. The amount of the opioid agonist, the opioid antagonist, or another therapeutic agent can refer to the weight of the salt or the weight of the free base of such agonist, antagonist or agent.

An amount of opioid antagonist that enhances the potency to alleviate a sign or symptom, such as the potency to alleviate pain intensity, stiffness, or difficulty physical function, of opioid agonist is the amount that when added to an analgesic or subanalgesic amount of agonist results upon administration in a greater alleviation (e.g., amelioration, attenuation, reduction, diminishment, blockage, inhibition or prevention) of at least one sign or symptom, such as pain, stiffness, or difficulty in physical function, than the alleviation of that sign or symptom resulting from administration of that agonist alone (i.e., without that amount of antagonist).

An amount of opioid antagonist that enhances the potency of an endogenous opioid agonist is the amount that when administered alone or with opioid agonist or another therapeutic agent, results in a greater alleviation (e.g., amelioration, attenuation, reduction, diminishment, blockage, inhibition or prevention) of at least one sign or symptom of pain than the alleviation of that sign or symptom without that amount of antagonist.

Opioids refer to compounds or compositions, including metabolites of the compounds or compositions, that bind to specific opioid receptors and have agonist (activation) or antagonist (inactivation) effects at the opioid receptors.

Inhibitory opioid receptors refer to opioid receptors that mediate inhibitory opioid receptor functions, such as analgesia.

Opioid receptor agonist or opioid agonist refers to an opioid compound or composition, including any active metabolite of such compound or composition, that binds to and activates opioid receptors on neurons that mediate pain.

An opioid receptor antagonist or opioid antagonist refers to an opioid compound or composition, including any active metabolite of such compound or composition, that binds to and blocks opioid receptors on neurons that mediate pain. An opioid antagonist attenuates (e.g., blocks, inhibits, prevents, or competes with) the action of an opioid agonist.

Pharmaceutically acceptable refers to those compounds, materials, compositions, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

Pharmaceutically acceptable salts refer to derivatives of the disclosed compounds wherein the compounds are modified by making at least one acid or base salt thereof, and includes inorganic and organic salts.

An analgesic amount refers of opioid agonist to an amount of the opioid agonist which causes analgesia in a patient administered the opioid receptor agonist alone, and includes standard doses of the agonist which are typically administered to cause analgesia (e.g. mg doses).

A subanalgesic amount of opioid agonist refers to an amount which does not cause analgesia in a patient administered the opioid receptor agonist alone, but when used in combination with a potentiating or enhancing amount of opioid antagonist, results in analgesia.

An effective antagonistic amount of opioid agonist refers to an amount that effectively attenuates (e.g. ameliorates, reduces, diminishes, blocks, inhibits, prevents, or competes with) the analgesic activity of an opioid agonist.

A therapeutically effective amount of a composition refers to an amount that elicits alleviation (e.g., amelioration, attenuation, reduction, diminishment, blockage, inhibition or prevention) of at least one sign or symptom of an arthritic condition, inflammation associated with a chronic condition, or chronic pain upon administration to a patient in need thereof.

Potency may refer to the strength of a drug or drug treatment in producing desired effects, for example, improved pain relief, improved pain control, reduced stiffness, and/or improved physical function. Potency also may refer to the effectiveness or efficacy of a drug treatment in eliciting desired effects, for example, improved pain relief, improved pain control, reduced stiffness, and/or improved physical function. For example, enhanced potency may refer to the lowering of a dose in achieving desired effects or to an increased therapeutic benefit including that not previously seen. In therapeutics, for example, potency may refer to the relative pharmacological activity of a compound or a composition.

The antagonist in the present compositions may be present in its original form or in the form of a pharmaceutically acceptable salt. The antagonists in the present compositions include: naltrexone, naloxone, nalmefene, methylnaltrexone, methiodide, nalorphine, naloxonazine, nalide, nalmexone, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, levorphanol, meptazinol, dezocine, or their pharmacologically effective esters or salts. Preferred opioid antagonists include naltrexone, nalmefene, naloxone, or mixtures thereof. Particularly preferred is nalmefene or naltrexone.

In general, for compositions, dosage forms, kits and methods according to the present invention, an opioid antagonist is provided in an amount from about 1 fg to about 1.0 mg or from about 1 fg to about 1 μg, including where the amount is provided by administration 1, 2, 3, or 4 times per day. Alternatively, the opioid antagonist is provided in an amount from at least about 0.000001 mg to about or less than about 0.5 or 1.0 mg, 0.00001 mg to about or less than about 0.5 or 1.0 mg, 0.0001 mg to about or less than about 0.5 or 1.0 mg, or at least about 0.001 mg to about or less than about 0.5 or 1.0 mg, or at least about 0.01 mg to about or less than about 0.5 or 1.0 mg, or at least about 0.1 mg to about or less than about 0.5 or 1.0 mg. Preferred ranges of opioid antagonists also include: from about 0.000001 mg to less than 0.2 mg; from about 0.00001 mg to less than 0.2 mg; from about 0.0001 mg to less than 0.2 mg; from about 0.001 mg to less than 0.2 mg; from about 0.01 mg to less than 0.2 mg; or from about 0.1 mg to less than 0.2 mg. Additional preferred ranges of opioid antagonists include: from about 0.0001 mg to about 0.1 mg; from about 0.001 mg to about 0.1 mg; from about 0.01 mg. to about 0.1 mg; from about 0.001 mg to about 0.1 mg; from about 0.001 mg to about 0.01 mg; or from about 0.01 mg to about 0.1 mg.

In a preferred dosage form, the maximum amount of antagonist is 1 mg, alternatively less than 1 mg, alternatively 0.99 mg, alternatively 0.98 mg, alternatively 0.97 mg, alternatively 0.96 mg, alternatively 0.95 mg, alternatively 0.94 mg, alternatively 0.93 mg, alternatively 0.92 mg, alternatively 0.91 mg, alternatively 0.90 mg, alternatively 0.89 mg, alternatively 0.88 mg, alternatively 0.87 mg, alternatively 0.86 mg, alternatively 0.85 mg, alternatively 0.84 mg, alternatively 0.83 mg, alternatively 0.82 mg, alternatively 0.81 mg, alternatively 0.80 mg, alternatively 0.79 mg, alternatively 0.78 mg, alternatively 0.77 mg, alternatively 0.76 mg, alternatively 0.75 mg, alternatively 0.74 mg, alternatively 0.73 mg, alternatively 0.72 mg, alternatively 0.71 mg, alternatively 0.70 mg, alternatively 0.69 mg, alternatively 0.68 mg, alternatively 0.67 mg, alternatively 0.66 mg, alternatively 0.65 mg, alternatively 0.64 mg, alternatively 0.63 mg, alternatively 0.62 mg, alternatively 0.61 mg, alternatively 0.60 mg, alternatively 0.59 mg, alternatively 0.58 mg, alternatively 0.57 mg, alternatively 0.56 mg, alternatively 0.55 mg, alternatively 0.54 mg, alternatively 0.53 mg, alternatively 0.52 mg, alternatively 0.51 mg, alternatively 0.50 mg.

Additionally, the maximum amount of antagonist in the dosage form is less than 0.5 mg, alternatively 0.49 mg, alternatively 0.48 mg, alternatively 0.47 mg, alternatively 0.46 mg, alternatively 0.45 mg, alternatively 0.44 mg, alternatively 0.43 mg, alternatively 0.42 mg, alternatively 0.41 mg, alternatively 0.40 mg, alternatively 0.39 mg, alternatively 0.38 mg, alternatively 0.37 mg, alternatively 0.36 mg, alternatively 0.35 mg, alternatively 0.34 mg, alternatively 0.33 mg, alternatively 0.32 mg, alternatively 0.31 mg, alternatively 0.30 mg, alternatively 0.29 mg, alternatively 0.28 mg, alternatively 0.27 mg, alternatively 0.26 mg, alternatively 0.25 mg, alternatively 0.24 mg, alternatively 0.23 mg, alternatively 0.22 mg, alternatively 0.21 mg, alternatively 0.20 mg, alternatively 0.19 mg, alternatively 0.18 mg, alternatively 0.17 mg, alternatively 0.16 mg, alternatively 0.15 mg, alternatively 0.14 mg, alternatively 0.13 mg, alternatively 0.12 mg, alternatively 0.11 mg, alternatively 0.10 mg, alternatively 0.09 mg, alternatively 0.08 mg, alternatively 0.07 mg, alternatively 0.06 mg, alternatively 0.05 mg, alternatively 0.04 mg, alternatively 0.03 mg, alternatively 0.02 mg, alternatively 0.01 mg, alternatively 0.009 mg, alternatively 0.008 mg, alternatively 0.007 mg, alternatively 0.006 mg, alternatively 0.005 mg, alternatively 0.004 mg, alternatively 0.003 mg, alternatively 0.002 mg, alternatively 0.001 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg.

The minimum amount of antagonist in the dosage form is 0.0001 mg, alternatively 0.0002 mg, alternatively 0.0003 mg, alternatively 0.0004 mg, alternatively 0.0005 mg, 0.0006 mg, alternatively 0.0007 mg, alternatively 0.0008 mg, alternatively 0.0009 mg, alternatively 0.001 mg, alternatively 0.002 mg, alternatively 0.003 mg, alternatively 0.004 mg, alternatively 0.005 mg, alternatively 0.006 mg, alternatively 0.007 mg, alternatively 0.008 mg, alternatively 0.009 mg, alternatively 0.01 mg, alternatively 0.011 mg, alternatively 0.012 mg, alternatively 0.013 mg, alternatively 0.014 mg, alternatively 0.015 mg, alternatively 0.016 mg, alternatively 0.017 mg, alternatively 0.018 mg, alternatively 0.019 mg, alternatively 0.02 mg, alternatively 0.021 mg, alternatively 0.022 mg, alternatively 0.023 mg, alternatively 0.024 mg, alternatively 0.025 mg, alternatively 0.026 mg, alternatively 0.027 mg, alternatively 0.028 mg, alternatively 0.029 mg, alternatively 0.03 mg, alternatively 0.031 mg, alternatively 0.032 mg, alternatively 0.033 mg, alternatively 0.034 mg, alternatively 0.035 mg, alternatively 0.036 mg, alternatively 0.037 mg, alternatively 0.038 mg, alternatively 0.039 mg, alternatively 0.04 mg, alternatively 0.041 mg, alternatively 0.042 mg, alternatively 0.043 mg, alternatively 0.044 mg, alternatively 0.045 mg, alternatively 0.046 mg, alternatively 0.047 mg, alternatively 0.048 mg, alternatively 0.049 mg, alternatively 0.05 mg, alternatively 0.051 mg, alternatively 0.052 mg, alternatively 0.053 mg, alternatively 0.054 mg, alternatively 0.055 mg, alternatively 0.056 mg, alternatively 0.057 mg, alternatively 0.058 mg, alternatively 0.059 mg, alternatively 0.06 mg, alternatively 0.061 mg, alternatively 0.062 mg, alternatively 0.063 mg, alternatively 0.064 mg, alternatively 0.065 mg, alternatively 0.066 mg, alternatively 0.067 mg, alternatively 0.068 mg, alternatively 0.069 mg, alternatively 0.07 mg, alternatively 0.071 mg, alternatively 0.072 mg, alternatively 0.073 mg, alternatively 0.074 mg, alternatively 0.075 mg, alternatively 0.076 mg, alternatively 0.077 mg, alternatively 0.078 mg, alternatively 0.079 mg, alternatively 0.08 mg, alternatively 0.081 mg, alternatively 0.082 mg, alternatively 0.083 mg, alternatively 0.084 mg, alternatively 0.085 mg, alternatively 0.086 mg, alternatively 0.087 mg, alternatively 0.088 mg, alternatively 0.089 mg, alternatively 0.09 mg, alternatively 0.091 mg, alternatively 0.092 mg, alternatively 0.093 mg, alternatively 0.094 mg, alternatively 0.095 mg, alternatively 0.096 mg, alternatively 0.097 mg, alternatively 0.098 mg, alternatively 0.099 mg, alternatively 0.1 mg, alternatively 0.11 mg, alternatively 0.12 mg, alternatively 0.13 mg, alternatively 0.14 mg, 0.15 mg, alternatively 0.16 mg, alternatively 0.17 mg, alternatively 0.18 mg, alternatively 0.19 mg, alternatively 0.2 mg, alternatively 0.21 mg, alternatively 0.22 mg, alternatively 0.23 mg, alternatively 0.24 mg, alternatively 0.25 mg, alternatively 0.26 mg, alternatively 0.27 mg, alternatively 0.28 mg, alternatively 0.29 mg, alternatively 0.3 mg, alternatively 0.31 mg, alternatively 0.32 mg, alternatively 0.33 mg, alternatively 0.34 mg, alternatively 0.35 mg, alternatively 0.36 mg, alternatively 0.37 mg, alternatively 0.38 mg, alternatively 0.39 mg alternatively 0.40 mg, alternatively 0.41 mg, alternatively 0.42 mg, alternatively 0.43 mg, alternatively 0.44 mg, alternatively 0.45 mg, alternatively 0.46 mg, alternatively 0.47 mg, alternatively 0.48 mg, alternatively 0.49 mg, alternatively 0.5 mg, alternatively 0.51 mg, alternatively 0.52 mg, alternatively 0.53 mg, alternatively 0.54 mg, alternatively 0.55 mg, alternatively 0.56 mg, alternatively 0.57 mg, alternatively 0.58 mg, alternatively 0.59 mg, alternatively 0.6 mg, alternatively 0.61 mg, alternatively 0.62 mg, alternatively 0.63 mg, alternatively 0.64 mg, alternatively 0.65 mg, alternatively 0.66 mg, alternatively 0.67 mg, alternatively 0.68 mg, alternatively 0.69 mg, alternatively 0.7 mg, alternatively 0.71 mg, alternatively 0.72 mg, alternatively 0.73 mg, alternatively 0.74 mg, alternatively 0.75 mg, alternatively 0.76 mg, alternatively 0.77 mg, alternatively 0.78 mg, alternatively 0.79 mg, alternatively 0.8 mg, alternatively 0.81 mg, alternatively 0.82 mg, alternatively 0.83 mg, alternatively 0.84 mg, alternatively 0.85 mg, alternatively 0.86 mg, alternatively 0.87 mg, alternatively 0.88 mg, alternatively 0.89 mg, alternatively 0.9 mg, alternatively 0.91 mg, alternatively 0.92 mg, alternatively 0.93 mg, alternatively 0.94 mg, alternatively 0.95 mg, alternatively 0.96 mg, alternatively 0.97 mg, alternatively 0.98 mg, alternatively 0.99 mg.

In a more preferred dosage form, the maximum amount of antagonist is less than 0.0020 mg, alternatively 0.0019 mg, alternatively 0.0018 mg, alternatively 0.0017 mg, alternatively 0.0016 mg, alternatively 0.0015 mg, alternatively 0.0014 mg, alternatively 0.0013 mg, alternatively 0.0012 mg, alternatively 0.0011 mg, alternatively 0.0010 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg, alternatively 0.0001 mg.

In a more preferred dosage form, the minimum amount of antagonist in the preferred dosage form is 0.0001 mg, alternatively 0.0002 mg, alternatively 0.0003 mg, alternatively 0.0004 mg, alternatively 0.0005 mg, alternatively 0.0006 mg, alternatively 0.0007 mg, alternatively 0.0008 mg, alternatively 0.0009 mg, alternatively 0.0010 mg, alternatively 0.0011 mg, alternatively 0.0012 mg, alternatively 0.0013 mg, alternatively 0.0014 mg, alternatively 0.0015 mg, alternatively 0.0016 mg, alternatively 0.0017 mg, alternatively 0.0018 mg, alternatively 0.0019 mg, alternatively 0.002 mg.

Any minimum amount and any maximum amount of antagonist in the dosage form, as specified above, may be combined to define a range of amounts, providing that the minimum selected is equal to or less than the maximum selected.

The amount of an opioid antagonist in the compositions for use in methods according to the present invention effective to enhance the potency of an opioid agonist can be less than an effective antagonistic amount. The effective amount of an opioid antagonist in the present compositions can be about 0.002 mg. The effective amount of an opioid antagonist in the present compositions can be less than 0.002 mg. The effective amount of an opioid antagonist in the present compositions can be about 0.001 mg. The effective amount of an opioid antagonist in the present compositions can be less than 0.001 mg. The effective amount of an opioid antagonist in the present compositions can be more than 0.0001 mg. The effective amount of an opioid antagonist in the present compositions can be about 0.0001 mg. The effective amount of an opioid antagonist in the present compositions can be about 0.00001 mg. The effective amount of an opioid antagonist in the present compositions can be less than 0.00001 mg. The effective amount of an opioid antagonist in the present compositions can be more than 0.00001 mg. The effective amount of an opioid antagonist in the present compositions can be about 0.000001 mg. The effective amount of an opioid antagonist in the present compositions can be less than 0.000001 mg. The effective amount of an opioid antagonist in the present compositions can be more than 0.000001 mg.

Any of the foregoing effective amounts may be administered one time per day, alternatively two times per day, alternatively three times per day, alternatively four times per day. Alternatively any of the following effective amounts may be divided over a series of dosages within one day or other relevant time period. For example, the effective amount may be divided into one, two, three or four doses administered over the day or other time period. Preferred effective amounts of an opioid antagonist include a total daily dose from about 0.00002 mg to about 0.002 mg, wherein the total daily dose is divided into 1, 2, 3, or 4 doses. For example, where the dose is administered two times per day, the opioid antagonist in preferably in an amount from about 0.00001 mg to about 0.001 mg in each of the two doses. Alternatively, where the dose is administered one time per day, the opioid antagonist in an amount from about 0.00002 mg to about 0.002 mg in the dose. Alternatively, where the dose is administered four times per day, the opioid antagonist in an amount from about 0.000005 mg to about 0.0005 mg in each of the four doses.

In the compositions for use in methods according to the present invention, the agonist may be present in its original form or in the form of a pharmaceutically acceptable salt. The agonists for use in methods according to the present invention include: alfentanil, allylprodine, alphaprodine, anileridine, apomorphine, apocodeine, benzylnorphine, bezitramide, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, narceine, nicomorphine, norlevorphanol, normethadone, nalorphine, normorphine, norpipanone, ohmefentanyl, opium, oxycodone, oxymorphone, papaveretum, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, salts thereof, mixtures of any of the foregoing, mixed mu-agonists/antagonists, mu-antagonist combinations, or others known to those skilled in the art. Preferred agonists for use in methods according to the present invention are morphine, hydrocodone, oxycodone, codeine, fentanyl (and its relatives), hydromorphone, meperidine, methadone, oxymorphone, propoxyphene or tramadol, or mixtures thereof. Particularly preferred contemplated agonists are morphine, hydrocodone, oxycodone or tramadol. Opioid agonists include exogenous or endogenous opioids. Endogenous opioid agonists include endorphin, beta-endorphin, enkephalin, met-enkephalin, dynorphin, orphanin FQ, neuropeptide FF, nociceptin, endomorphin, endormorphin-1, endormorphin-2.

The agonist may be present in an amount that is analgesic or subanalgesic (e.g., non-analgesic) in the human subject. The agonist is administered in dosage forms containing from about 0.1 to about 300 mg of agonist, alternatively from about 2.5 to about 160 mg of agonist. The agonist, in conjunction with antagonist, is included in the dosage form in an amount sufficient to produce the desired effect upon the process or condition of pain, including inflammatory pain, such as alleviation (e.g., amelioration, attenuation, reduction, diminishment, blockage, inhibition or prevention) of at least one symptom of pain, including inflammatory pain. Symptoms and signs include, for example, pain (including chronic pain), stiffness or difficulty in physical function.

Preferred combinations of an opioid antagonist and opioid agonist in the present compositions are naltrexone and oxycodone; naltrexone and oxymorphone; naltrexone and hydrocodone; naltrexone and hydromorphone; naltrexone and morphine; nalmefene and oxycodone; nalmefene and oxymorphone; nalmefene and hydrocodone; nalmefene and hydromorphone; nalmefene and morphine; naloxone and oxycodone; naloxone and oxymorphone; naloxone and hydrocodone; naloxone and hydromorphone; and naloxone and morphine, respectively.

The more preferred combinations of an opioid antagonist and opioid agonist in the present compositions are naltrexone and oxycodone; naltrexone and oxymorphone; naltrexone and hydrocodone; naltrexone and hydromorphone; naltrexone and morphine; nalmefene and oxycodone; nalmefene and oxymorphone; nalmefene and hydrocodone; nalmefene and hydromorphone; and nalmefene and morphine, respectively.

The most preferred combinations of an opioid antagonist and opioid agonist in the present compositions are naltrexone and oxycodone; naltrexone and oxymorphone; naltrexone and hydrocodone; naltrexone and hydromorphone; and naltrexone and morphine, respectively.

In an embodiment of the invention, the amount of antagonist in the dosage form is less than an effective amount to antagonize an exogenous or endogenous agonist, but such an amount is effective to enhance the pain-enhancing potency, including the inflammatory pain-enhancing potency, of the agonist and optionally but preferably is effective to attenuate an adverse effect of the agonist, for example, tolerance, withdrawal, dependence and/or addiction. In another aspect of the invention, the method further comprises administering the opioid agonist, in either a combined dosage form with the antagonist or in a separate dosage form. Still another aspect of the invention provides an immediate release solid oral dosage form comprising one or more pharmaceutical excipients, a dose of an opioid agonist and a low dose of an opioid antagonist, wherein the opioid agonist and opioid antagonist are release concurrently when placed in an aqueous environment. The opioid antagonist and opioid agonist can be formulated as immediate release, (IR), controlled release (CR) and/or sustained released (SR) formulations. Formulations can have components that are combinations of IR and/or CR and/or SR components.

The combination dosage forms of the present compositions can be formulated to provide a concurrent release of the opioid antagonist in combination with opioid agonist and/or other therapeutic agent generally throughout at least a majority of the delivery profile for the formulation. As used herein, the terms “concurrent release” and “released concurrently” mean that the agonist and antagonist are released in in vitro dissolution assays in an overlapping manner. The respective beginnings of release of each agent can but need not necessarily be simultaneous. Concurrent release will occur when the majority of the release of the first agent overlap a majority of release of the second agent. A desired portion of each active pharmaceutical ingredient may be released within a desired time. The desired portions may be, for example, 5%, 50% or 90%, or some other percentage between 1% and 100%. The desired time may be in minutes or hours, for example, 10 minutes, 20 minutes, 30 minutes, or 45 minutes, or some other time. The desired portion and the desired time may be varied by the inclusion of formulants for the controlled release or sustained release of any therapeutic agent(s).

The optimum amounts of the opioid antagonist administered in combination with an opioid agonist or other therapeutic agent will of course depend upon the particular antagonist and agonist or other agent used, the excipient chosen, the route of administration, and/or the pharmacokinetic properties of the patient being treated. Effective administration levels of antagonist and agonist or other agent will vary upon the state and circumstances of the patient being treated. As those skilled in the art will recognize, many factors that modify the action of an active ingredient will be taken into account by a treating physician, such as the age, body weight, sex, diet, and condition of the patient, the lapse of time between the condition or injury and the administration of the present compositions, and the administration technique. A person of ordinary skill in the art will be able to ascertain the optimal dosage for a given set of conditions in view of the disclosure herein.

The opioid agonist and/or antagonist can be present in the present compositions as an acid, base, pharmaceutically acceptable salt, or a combination thereof. The pharmaceutically acceptable salt embraces inorganic or organic salts. Examples of pharmaceutically acceptable salts include, but are not limited to, mineral or organic acid salts. The pharmaceutically acceptable salts include the conventional non-toxic salts made, for example, from non-toxic inorganic or organic acids. For example, such conventional non-toxic salts include those derived from inorganic acids such as hydrochloric, hydrobromic, sulfuric, sulfonic, sulfamic, phosphoric, nitric and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucuronic, and other acids. Other pharmaceutically acceptable salts and variants include mucates, phosphate (dibasic), phosphate (monobasic), acetate trihydrate, bi(heptafluorobutyrate), bi(methylcarbamate), bi(pentafluoropropionate), mesylate, bi(pyridine-3-carboxylate), bi(trifluoroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are all incorporated herein by reference. Additional representative salts include hydrobromide, hydrochloride, mucate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate, bi(pyridine-3-carboxylate), bi(trifluoroacetate), bitartrate, chlorhydrate, fumarate, and sulfate pentahydrate.

The methods may further comprise administering to the subject another therapeutic agent, for example, non-steroidal anti-inflammatory drug agents or local anesthetic and/or analgesic agents, TNF-α antagonists, corticosteroids, disease-modifying anti-rheumatic drugs (DMARDs), anticonvulsant agents, tricyclic antidepressant agents, anti-dynorphin agents, glutamate receptor antagonist agents. In particularly, it is specifically completed that, in addition to the opioid agonist and the opioid antagonist, the subject may be administered TNF-α antagonists, P38 inhibitors, and cytokines inhibitors (including but not limited to IL-2, IL-6, IL-8, and GM-CSF). The opioid agonist, the opioid antagonist, and other therapeutic agent may be administered to the subject in a combined dosage form.

An NSAID refers to a non-steroidal anti-inflammatory drug and includes anti-inflammatory drugs such as aspirin, members of the cycloxgenease I, II and III inhibitors, and includes naproxen sodium, diclofenac and misoprostol, valdecoxib, diclofenac, celecoxib, sulindac, oxaprozin, diflunisal, piroxicam, indomethacin, meloxicam, ibuprofen, naproxen, mefenamic acid, nabumetone, ketorolac, choline or magnesium salicylates, rofecoxib, tolmetin sodium, phenylbutazone, oxyphenbutzone, meclofenamate sodium or diflusenal.

In an embodiment, the present compositions further comprise at least one non-narcotic analgesic, such as a nonsteroidal anti-inflammatory agent (NSAID). Representative nonsteroidal anti-inflammatory agents include aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxican, sulindac, tolmetin, and zomepirac. Currently marketed NSAIDs include Celebrex®, Vioxx®, Anaprox®, Arthrotec®, Bextra®, Cataflam®, Clinoril®, DayPro®, Dolobid®, Feldene®, Indocin®, Mobic®, Motrin®, Negprelen®, Naprosyn®, Ponstel®, Relafen®, Toradol®.

In an embodiment, the present compositions may further comprise an analgesic, antipyretic, and/or anti-inflammatory therapeutic agent. For example, the composition may further comprise one or more of aspirin, sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, refecoxib, celecoxib, etodolac, and nimesulide.

With regard to dosage levels, the non-narcotic analgesic is present in a inflammatory pain-alleviating amount or an amount that is not pain-alleviating alone but is pain-alleviating in combination with an opioid agonist and opioid antagonist according to the invention. This amount is at a level corresponding to the generally recommended adult human dosages for a particular non-narcotic analgesic. The effective inflammatory pain-alleviating amount of the opioid antagonist and the opioid agonist can be present at a level that potentiates the inflammatory pain-alleviating effectiveness of the non-narcotic analgesic. Specific dosage levels for the non-narcotic analgesic that can be used herein as given, inter alia, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of all are incorporated herein by reference. As is well known to one of ordinary skill in the art, there can be a wide variation in the dosage level of the non-narcotic analgesic, wherein the dosage level depends to a large extent on the specific non-narcotic analgesic being administered. These amounts can be determined for a particular drug combination in accordance with this invention by employing routine experimental testing.

In an embodiment, the present compositions further comprise at least one inhibitor of TNF-α. Inhibitors of TNF-α may also be designated TNF-α antagonists. TNF-α antagonists are compounds which are capable of, directly or indirectly, counteracting, reducing or inhibiting the biological activity of TNF-α, or the activation of receptors therefore. Tumor necrosis factor (TNF) is a key proinflammatory cytokine released by a number of cell types, particularly activated macrophages and monocytes. Two forms of TNF are released—TNF-α and TNF-beta. TNF-α is a soluble homotrimer of 17 kD protein subunits (Smith et al., J. Biol. Chem. 262:6951-6954 (1987). A membrane-bound 26 kD precursor form of TNF also exists as a pro-protein and must be cleaved to produce the 17 kD TNF. (Kriegler, et al., Cell, 53 :45-53 (1988). Without limitation, the TNF-α antagonist can be a compound that affects the synthesis of TNF-α, or one that affects the maturation of TNF-α, or one that inhibits the binding of TNF-α with a receptor specific for TNF-α, or one that interferes with intracellular signaling triggered by TNFα binding with a receptor. Additional details regarding the manufacture and use of TNF-α antagonists are available in U.S. Patent Application Publication No. U.S. 2003/0157061 A1, which is incorporated herein by reference.

Preferred TNF-α antagonists for the present invention include ENBREL® (etanercept) from Wyeth-Ayerst Laboratories/Immunex; REMICADE®, infiximab, which is an anti-TNF chimeric Mab (Centocor; Johnson & Johnson); anti-TNF-α, D2E7 human Mab (Cambridge antibody Technology); CDP-870, which is a PEGylated antibody fragment (Celltech); CDP-571; Humicade, which is a humanized Mab described in U.S. Pat. No. 5,994,510 (Celltech); PEGylated soluble TNF-α Receptor-1 (Amgen); TBP-1, which is a TNF binding protein (Ares Serono); PASSTNF-alpha®, which is an anti-TNF-α polyclonal antibody (Verigen); AGT-1, which is a mixture of three anti-cytokine antibodies to IFN-alpha, IFN-gamma, and TNF (Advanced Biotherapy Concepts); TENEFUSE®, ienercept, which is a TNFR-Ig fusion protein (Roche); CytoTAB® (Protherics); TACE, which is a small molecule TNF-α converting enzyme inhibitor (immunex); small molecule TNF mRNA synthesis inhibitor (Nereus); PEGylated p75TNFR Fc mutein (Immunex); and TNF-α antisense inhibitor.

With regard to dosage levels, the TNF-α antagonist is present at an amount effective to inhibit progression or reduce damage from an arthritic condition or a chronic condition associated with inflammation. Alternatively, the TNF-α antagonist is present in an amount that is not effective to inhibit progression or reduce damage alone but is effective to inhibit progression or reduce damage in combination with an opioid agonist and opioid antagonist according to the invention. This amount is at a level corresponding to the generally recommended adult human dosages for a particular TNF-α antagonist. The effective pain-alleviating amount of the opioid antagonist and the opioid agonist can be present at a level that potentiates the effectiveness of a TNF-α antagonist. Specific dosage levels for TNF-α antagonists that can be used herein as given, inter alia, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosure of all are incorporated herein by reference. As is well known to one of ordinary skill in the art, there can be a wide variation in the dosage level of the TNF-α antagonist, wherein the dosage level depends to a large extent on the specific TNF-α antagonist being administered. These amounts can be determined for a particular drug combination, in accordance with this invention, by employing routine experimental testing.

In an embodiment, the present compositions further comprise at least one anti-rheumatic drug. Anti-rheumatic drugs include those referred to as Disease-modifying antirheumatic drugs (DMARDs). Anti-rheumatic drugs include methotrexate (RHEUMATREX, TREXALL), leflunomide (ARAVA), D-Penicillamine, sulfasalazine, gold therapy, minocycline, azathioprine, hydroxychloroquine (PLAQUENIL) and other antimalarials, cyclosporine and biologic agents. Biologic response modifiers, often referred to as biologic agents or simply biologics, are designed to either inhibit or supplement immune system components called cytokines. Cytokines play a role in either fueling or suppressing the inflammation that causes damage in RA and some other diseases. The four biologics currently approved for RA all work by inhibiting inflammatory cytokines. Adalimumab (HUMIRA), etanercept (ENBREL) and infliximab (REMICADE) work to inhibit a cytokine called tumor necrosis factor (TNF). Anakinra (KINERET) blocks the action of the cytokine interleukin-1 (IL-1).

With regard to dosage levels, the anti-rheumatic drug is present at an amount that attenuates a symptom or sign of rheumatism or an amount that does not attenuate such a symptom or sign alone but does attenuate such a symptom or sign in combination with an opioid agonist and opioid antagonist according to the invention. This amount is at a level corresponding to the generally recommended adult human dosages for a particular anti-rheumatic drug. The effective amount of the opioid antagonist and the opioid agonist can be present at a level that potentiates the effectiveness of the anti-rheumatic drug. Specific dosage levels for anti-rheumatic drugs that can be used herein as given, inter alia, are included, for example, in the “Physicians' Desk Reference”, 2003 Edition (Medical Economics Data Production Company, Montvale, N.J.) as well as in other reference works.

In an embodiment, the present compositions further comprise at least one anticonvulsant or anti-epileptic agent. Any therapeutically effective anticonvulsant may be used according to the invention. For extensive listings of anticonvulsants, see, e.g., Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083 (the disclosures of which are incorporated herein by reference). Representative anticonvulsants that can be used herein include lamotrigine, gabapentin, valproic acid, topiramate, famotodine, phenobarbital, diphenylhydantoin, phenyloin, mephenyloin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenyloin sodium, valproate sodium, clobazam, sulthiame, dilantin, diphenylan and L-5-hydroxytryptophan. Currently marketed anticonvulant/anti-epileptic drugs include Keppra®, Lamictol®, Neurontin®, Tegretol®, Carbatrol®, Topiramate®, Trileptal®, and Zonegran®.

With regard to dosage levels, the anticonvulsant is present at a pain-alleviating amount or an amount that is not pain-alleviating alone but is pain-alleviating in combination with an opioid agonist and opioid antagonist according to the invention. This amount is at a level corresponding to the generally recommended adult human dosages for a particular anticonvulsant. The effective pain-alleviating amount of the opioid antagonist and the opioid agonist can be present at a level that potentiates the pain-alleviating effectiveness of the anticonvulsant. Specific dosage levels for anticonvulsants that can be used herein as given, inter alia, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosure of all are incorporated herein by reference. As is well known to one of ordinary skill in the art, there can be a wide variation in the dosage level of the anticonvulsant, wherein the dosage level depends to a large extent on the specific anticonvulsant being administered. These amounts can be determined for a particular drug combination, in accordance with this invention, by employing routine experimental testing.

The compositions presented herein may be compounded, for example, with the usual non-toxic, pharmaceutically acceptable excipients, carriers, diluents or other adjuvants. The choice of adjuvants will depend upon the active ingredients employed, the physical form of the composition, the route of administration, and other factors.

The excipients, binders, carriers, and diluents which can be used include water, glucose, lactose, natural sugars such as sucrose, glucose, or corn sweeteners, sorbitol, natural and synthetic gums such as gum acacia, tragacanth, sodium alginate, and gum arabic, gelatin, mannitol, starches such as starch paste, corn starch, or potato starch, magnesium trisilicate, talc, keratin, colloidal silica, urea, stearic acid, magnesium stearate, dibasic calcium phosphate, crystalline cellulose, methyl cellulose, carboxymethyl cellulose, polyethylene glycol, waxes, glycerin, and saline solution, among others.

Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The dosage forms can also comprise one or more acidifying agents, adsorbents, alkalizing agents, antiadherents, antioxidants, binders, buffering agents, colorants, complexing agents, diluents or fillers, direct compression excipients, disintegrants, flavorants, fragrances, glidants, lubricants, opaquants, plasticizers, polishing agents, preservatives, sweetening agents, or other ingredients known for use in pharmaceutical preparations.

Acidifying agents are a compound used to provide an acidic medium for product stability. Such compounds include, by way of example and without limitation, acetic acid, amino acid, citric acid, fumaric acid and other alpha hydroxy acids, hydrochloric acid, ascorbic acid, nitric acid, phosphoric acid, and others known to those skilled in the art.

Adsorbents are agents capable of holding other molecules onto their surface by physical or chemical (chemisorption) means. Such compounds include, by way of example and without limitation, powdered and activated charcoal, zeolites, and other materials known to one of ordinary skill in the art.

Alkalizing agent are compounds used to provide an alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those skilled in the art.

Antiadherent are agents that prevents the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

Antioxidants are agents which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

Binders are substances used to cause adhesion of powder particles in solid dosage formulations. Such compounds include, by way of example and without limitation, acacia, alginic acid, carboxymethylcellulose sodium, poly(vinylpyrrolidone), compressible sugar (e.g., NuTab), ethylcellulose, hydroxypropyl methylcellulose, gelatin, liquid glucose, methylcellulose, povidone and pregelatinized starch and other materials known to one of ordinary skill in the art.

When needed, binders may also be included in the dosage forms. Exemplary binders include acacia, tragacanth, gelatin, starch, cellulose materials such as methyl cellulose, HPMC, HPC, HEC and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, gelatin, cellulosics in nonaqueous solvents, combinations thereof and others known to those skilled in the art. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

Buffering agents are compounds used to resist changes in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

Sweetening agents are compounds used to impart sweetness to a preparation. Such compounds include, by way of example and without limitation, aspartame, dextrose, glycerin, mannitol, saccharin sodium, sorbitol, sucrose, and other materials known to one of ordinary skill in the art.

Diluents or fillers are inert substances used to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, and starch and other materials known to one of ordinary skill in the art.

Direct compression excipients are compounds used in compressed solid dosage forms. Such compounds include, by way of example and without limitation, dibasic calcium phosphate (e.g., Ditab) and other materials known to one of ordinary skill in the art.

Disintegrants are compounds used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays such as bentonite, low substituted hydroxypropyl cellulose, microcrystalline cellulose (e.g., Avicel), methyl cellulose, carboxymethylcellulose calcium, sodium carboxymethylcellulose, alginic acid, sodium alginate, cellulose polyacrilin potassium (e.g., Amberlite), alginates, sodium starch glycolate, gums, agar, guar, locust bean, karaya, xanthan, pectin, tragacanth, agar, bentonite, and other materials known to one of ordinary skill in the art.

Glidants are agents used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art.

Lubricants are substances used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art.

Opaquants are compounds used to render a coating opaque. An opaquant may be used alone or in combination with a colorant. Such compounds include, by way of example and without limitation, titanium dioxide, talc and other materials known to one of ordinary skill in the art.

Polishing agents are compounds used to impart an attractive sheen to solid dosage forms. Such compounds include, by way of example and without limitation, carnauba wax, white wax and other materials known to one of ordinary skill in the art.

Colorants are compounds used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, ferric oxide, other FD&C dyes and natural coloring agents such as grape skin extract, beet red powder, beta-carotene, annato, carmine, turmeric, paprika, and other materials known to one of ordinary skill in the art. The amount of coloring agent used will vary as desired.

Flavorants are compounds used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those skilled in the art. Particularly contemplated flavors are the grape and cherry flavors and citrus flavors such as orange.

Complexing agents include for example EDTA disodium or its other salts and other agents known to one of ordinary skill in the art.

Exemplary fragrances include those generally accepted as FD&C grade.

Exemplary preservatives include materials that inhibit bacterial growth, such as Nipagin, Nipasol, alcohol, antimicrobial agents, benzoic acid, sodium benzoate, benzyl alcohol, sorbic acid, parabens, isopropyl alcohol and others known to one of ordinary skill in the art.

Solid dosage forms of the invention can also employ one or more surface active agents or cosolvents that improve wetting or disintegration of the core and/or layer of the solid dosage form.

Plasticizers can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. The PEG based plasticizers are available commercially or can be made by a variety of methods, such as disclosed in Poly(ethylene glycol) Chemistry: Biotechnical and Biomedical Applications (J.M. Harris, Ed.; Plenum Press, NY) the disclosure of which is hereby incorporated by reference.

Solid dosage forms of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for the solid pharmaceutical compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl beta-aminopropionates and 2-alkylimidazoline quaternary ammonium salts; and others known to one of ordinary skill in the art; and mixtures thereof.

A water soluble coat or layer can be formed to surround a solid dosage form or a portion thereof. The water soluble coat or layer can either be inert or drug-containing. Such a coat or layer will generally comprise an inert and non-toxic material which is at least partially, and optionally substantially completely, soluble or erodible in an environment of use. Selection of suitable materials will depend upon the desired behavior of the dosage form. A rapidly dissolving coat or layer will be soluble in the buccal cavity and/or upper GI tract, such as the stomach, duodenum, jejunum or upper small intestines. Exemplary materials are disclosed in U.S. Pat. No. 4,576,604 to Guittard et al. and No. 4,673,405 to Guittard et al., and No. 6,004,582 to Faour et al. and the text Pharmaceutical Dosage Forms: Tablets Volume I, 2^{nd} Edition. (A. Lieberman. ed. 1989, Marcel Dekker, Inc.), the disclosures of which are hereby incorporated by reference. In some embodiments, the rapidly dissolving coat or layer will be soluble in saliva, gastric juices, or acidic fluids.

Materials which are suitable for making the water soluble coat or layer include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those skilled in the art. Other lamina-forming materials that can be used for this purpose include poly(vinyl alcohol), poly(ethylene oxide), gelatin, glucose and saccharides. The water soluble coating can comprise other pharmaceutical excipients that may or may not alter the way in which the water soluble coating behaves. The artisan of ordinary skill will recognize that the above-noted materials include film-forming polymers.

A water soluble coat or layer can also comprise hydroxypropyl methylcellulose, which is supplied by Dow under its Methocel E-15 trademark. The materials can be prepared in solutions having different concentrations of polymer according to the desired solution viscosity. For example, a 2% W/V aqueous solution of Methocel™ E-15 has a viscosity of about 13-18 cps at 20° C.

For transcutaneous or transdermal administration, the compounds may be combined with skin penetration enhancers such as propylene glycol, polyethylene glycol, isopropanol, ethanol, oleic acid, N-methylpyrrolidone, or others known to those skilled in the art, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, or others known to those skilled in the art, to provide the composition in gel form, which can be dissolved in solvent such as methylene chloride, evaporated to the desired viscosity, and then applied to backing material to provide a patch.

For intravenous, intramuscular, subcutaneous, intrathecal, epidural, perineural or intradermal administration, the active ingredients may be combined with a sterile aqueous solution. The solution may be isotonic with the blood of the recipient. Such formulations may be prepared by dissolving one or more solid active ingredients in water containing physiologically compatible substances such as sodium chloride, glycine, or others known to those skilled in the art, and/or having a buffered pH compatible with physiological conditions to produce an aqueous solution, and/or rendering the solution sterile. The formulations may be present in unit dose containers such as sealed ampoules or vials.

For topical (e.g., dermal or subdermal) or depot administration, the active ingredients may be formulated with oils such as cottonseed, hydrogenated castor oil and mineral oil; short chain alcohols as chlorobutanol and benzyl alcohol; also including polyethylene glycols, polysorbates; polymers such as sucrose acetate isobutyrate, caboxymethocellusose and acrylates; buffers such as dihydrogen phosphate; salts such as sodium chloride and calcium phosphate; and other ingredients included but not exclusive to povidone, lactose monohydrate, magnesium stearate, myristyo-gamma-picolinium; and water.

A solid dosage form of the invention can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

Various other components, in some cases not otherwise listed above, can be added to the present formulation for optimization of a desired active agent release profile including, by way of example and without limitation, glycerylmonostearate, nylon, cellulose acetate butyrate, d,l-poly(lactic acid), 1,6-hexanediamine, diethylenetriamine, starches, derivatized starches, acetylated monoglycerides, gelatin coacervates, poly (styrene—maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

The compositions for use in the methods of the present invention can be formulated in capsules, tablets, caplets, or pills. Such capsules, tablets, caplets, or pills of the present inflammatory pain-alleviating compositions can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Similarly, the carrier or diluent may include any sustained release material known in the art, such as glyceryl monostearate or glyceryl distearate, alone or mixed with a wax. The formulations of the invention may be formulated so as to provide quick, sustained, or delayed release of the active ingredient after administration to the patient by employing procedures well known in the art.

Controlled release or sustained-release dosage forms, as well as immediate release dosage forms are specifically contemplated. Controlled release or sustained release as well as immediate release compositions in liquid forms in which a therapeutic agent may be incorporated for administration orally or by injection are also contemplated.

The pharmaceutical compositions or dosage forms of this invention may be used in the form of a pharmaceutical preparation which contains one or more opioid antagonists in combination with one or more opioid agonists.

It has been previously discovered that some opioid antagonists undesirably bind significantly to certain pharmaceutical excipients. Those pharmaceutical excipients generally cause an incomplete amount of the opioid antagonist to be released from a dosage form, within a particular time allotted for release. For example, when naltrexone hydrochloride in solution was mixed with croscarmellose sodium in suspension, the croscarmellose sodium bound more than 90% of the naltrexone hydrochloride. Accordingly, opioid antagonists must be tested with pharmaceutical excipients, so as to ensure that the excipient does not bind the opioid antagonist to a significant degree. Excipients, for example, binders, disintegrants, glidants, lubricants, or acidifiers, as well as process conditions, such as pH, should be selected with this in mind.

The compositions present herein for alleviating the symptoms or signs of arthritic conditions, chronic conditions associated with inflammation or chronic pain can be administered from about one time daily to about six times daily, two times daily to about four times daily, or one time daily to about two times daily.

Pain-alleviating compositions, including inflammatory pain-alleviating compositions, presented herein preferably comprise at least one colloidal dispersion system, additive or preservative, diluent, binder, plasticizer, or slow release agent.

It should be understood that compounds used in the art of pharmaceutical formulation generally serve a variety of functions or purposes. Thus, whether a compound named herein is mentioned only once or is used to define more than one term herein, its purpose or function should not be construed as being limited solely to the named purpose(s) or function(s).

The present pain-alleviating compositions, including inflammatory pain-alleviating compositions, may be in admixture with an organic or inorganic carrier or excipient suitable for administration in enteral or parenteral applications, such as orally, topically, transdermally, by inhalation spray, rectally, by subcutaneous, intravenous, intramuscular, subcutaneous, intrathecal, epidural, perineural, intradermal, intraocular injection or infusion techniques. Preferably, such compositions are in the form of a topical, intravenous, intrathecal, epidural, perineural, or oral formulation. More preferably, such compositions are in the form of an intrathecal, epidural or perineural formulation. Even more preferably, such compositions are in the form of an intravenous formulation. Most preferably, such compositions are in the form of an oral formulation.

The present invention is additionally advantageous because it can be used to enhance (e.g., increase) analgesic potency of the opioid agonists without substantially increasing the adverse side effects in humans associated with that dose of agonist. For example, the present methods and compositions may be employed in human subjects without significant increases in incidents of eye disorders, gastrointestinal disorders (such as upper abdominal pain, constipation, diarrhea, nausea, and vomiting), general disorders and conditions (such as lethargy), nervous system disorders (such as dizziness, headache, sedation, and sommolence), psychiatric disorders (such as euphoric mood), and skin and subcutaneous tissue disorders (such as pruritus). For compositions and methods of the invention that enhance analgesic potency of the opioid agonist, it is advantageous that adverse side effects are not increased with that enhanced (e.g., increased) potency.

The following examples are provided for illustrative purposes and are not to be construed to limit the scope of the claims in any manner whatsoever.

### EXAMPLE 1

A.

In a clinical study, the effects of an exemplary opioid agonist oxycodone in combination with an exemplary opioid antagonist naltrexone were evaluated in subjects with moderate to severe chronic pain due to an exemplary arthritic condition osteoarthritis of the hip or knee.

A clinical study was designed as follows: (1) to evaluate the efficacy and safety of combinations of oxycodone (oxy) and naltrexone (ntx) administered twice daily and four times daily relative to oxycodone administered four times daily while maintaining the same total daily oxycodone dose, and (2) to evaluate the frequency and severity of opioid withdrawal in patients who received combinations of oxycodone and naltrexone compared to those patients who received oxycodone.

A multicenter, randomized, double-blind, active- and placebo-controlled, dose escalation, clinical study was designed and conducted. The study evaluated the efficacy and safety of an oral formulation of oxycodone and naltrexone relative to oxycodone over a 3-week period in patients with chronic pain due to osteoarthritis of the hip or knee. A total of 360 patients were enrolled into four treatment groups: two groups for combinations of oxycodone and naltrexone, a group for oxycodone alone, and a group for placebo. During a 4- to 7-day washout period, patients stopped taking all of their pain medication other than acetaminophen (500 mg every 4-6 hours PRN (a maximum of 5 caplets per day)).

A daily diary was to be utilized to record overall pain intensity (PI) and other signs and symptoms. The patient was enrolled in the study if: (1) the mean value of the diary PI over the last 2 days of the 4- to 7-day baseline period was ≧5; (2) the confirmatory PI obtained at the baseline clinic visit was also≧5; and, (3) the patient met all inclusion/exclusion criteria. Baseline functional assessments were conducted with the SF-12 Health Survey as shown in Table 1 and the Western Ontario and MacMaster Universities Osteoarthritis Index (WOMAC) as shown in Table 2 below before the initiation of study medication.

TABLE 1
The SF-12v2 ™ Health Survey

Instructions for Completing the Questionnaire
Please answer every question. Some questions may look like others, but each one is different. Please take the
time to read and answer each question carefully by filling in the bubble that best represents your response.
EXAMPLE
This is for your review. Do not answer this question. The questionnaire begins with the section Your Health in
General below.
For each question you will be asked to fill in a bubble in each line:

| | |
|---|---|
| 1. | How strongly do you agree or disagree with each of the following statements? |

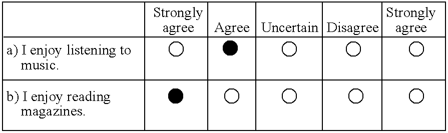

Please begin answering the questions now.

Your Health in General

| | |
|---|---|
| 1. | In general, would you say your health is: |

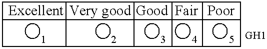

| | |
|---|---|
| 2. | The following questions are about activities you might do during a typical day. Does your health now |
| | limit you in these activities? If so, how much? |

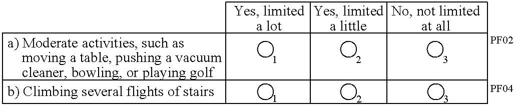

| | |
|---|---|
| 3. | During the past week, how much of the time have you had any of the following problems with your |
| | work or other regular daily activities as a result of your physical health? |

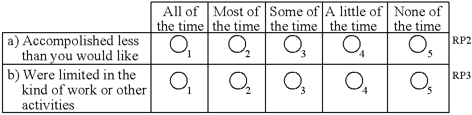

| | |
|---|---|
| 4. | During the past week, how much of the time have you had any of the following problems with your |
| | work or other regular daily activities as a result of any emotional problems (such as feeling |
| | depressed or anxious)? |

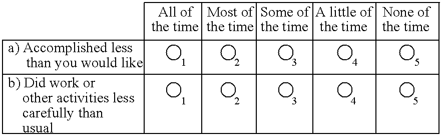

| | |
|---|---|
| 5. | During the past week, how much did pain interfere with your normal work (including both work |
| | outside the home and housework)? |

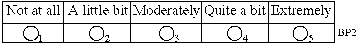

| | |
|---|---|
| 6. | These questions are about how you feel and how things have been with you during the past week. |
| | For each question, please give the one answer that comes closest to the way you have been feeling. |
| | How much of the time during the past week . . . |

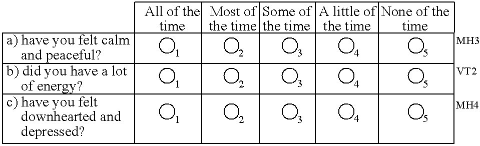

| | |
|---|---|
| 7. | During the past week, how much of the time has your physical health or emotional problems |
| | interfered with your social activities (like visiting friends, relatives, etc.)? |

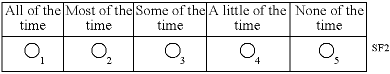

THANK YOU FOR COMPLETING THIS QUESTIONNAIRE!

TABLE 2
WOMAC OSTEOARTHRITIS INDEX

Directions: Please refer to the instructions provided to you for completion of the following
questions.

Section A
PAIN

Think about the pain you felt in your_{—————}(study joint) caused by your arthritis during
the last 48 hours. (Please mark you answers with an “x”).

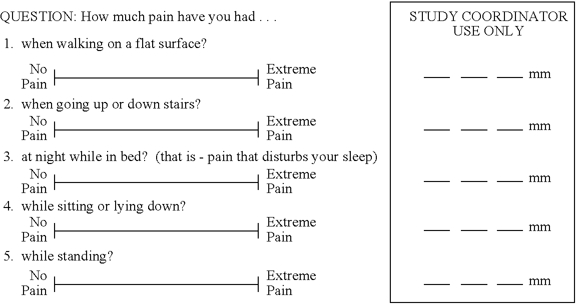

Section B
STIFFNESS

Think about the stiffness (not pain) felt in your_{—————}(study joint) caused by your
arthritis during the last 48 hours. Stiffness is a sensation of decreased ease in moving your joint.
(Please mark your answers with an “x”).

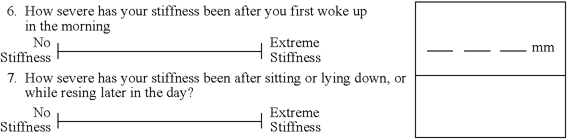

Section C
DIFFICULTY PERFORMING DAILY ACTIVITIES

Think about the difficulty you had in doing the following daily physical activities caused by your
arthritis in your_{—————}(study joint) during the last 48 hours. By this we mean your
ability to move around and take care of yourself. (Please mark you answers with an “x”).

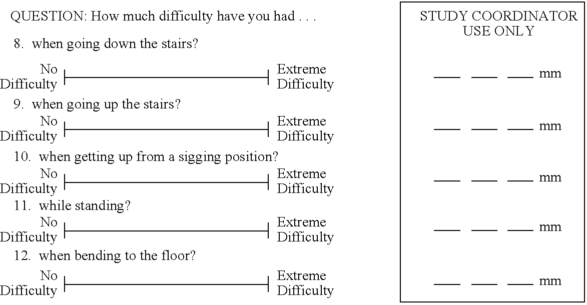

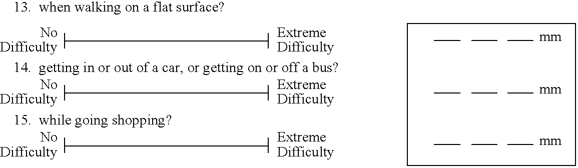

Think about the difficulty you had the following daily physical activities caused by your
arthritis in your_{—————}(study joint) during the last 48 hours. By this we mean your
ability to move around and take care of yourself. (Please mark your answers with an “x”).

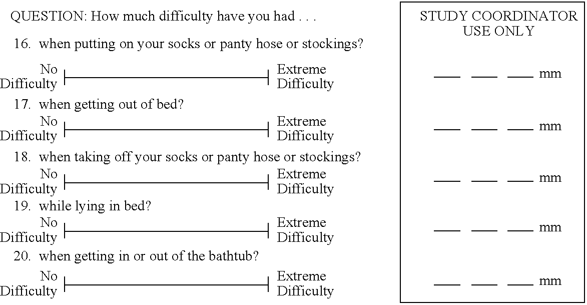

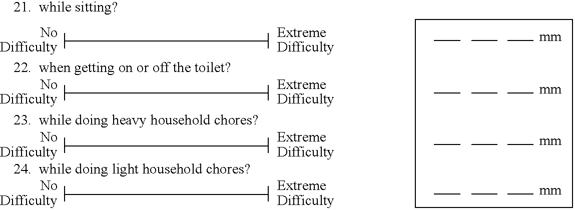

Patients were randomly assigned to one of the four treatment groups as shown in Table 3.

TABLE 3

| Treatment | Week 1 | | Week 2 | Week 3 |
|---|---|---|---|---|
| Group | (Days 1-3) | (Days 4-8) | (Days 1-8) | (Days 1-8) |
| A | (OXY 5 mg + NTX | (OXY 10 mg + NTX | (OXY 15 mg + NTX | (OXY 20 mg + NTX |
| | 0.001 mg) BID | 0.001 mg) BID | 0.001 mg) BID | 0.001 mg) |
| B | (OXY 2.5 mg + NTX | (OXY 5 mg + NTX | (OXY 7.5 mg + NTX | (OXY 10 mg + NTX |
| | 0.001 mg) QID | 0.001 mg) QID | 0.001 mg) QID | 0.001 mg) QID |
| C | OXY 2.5 mg QID | OXY 5 mg QID | OXY 7.5 mg QID | OXY 10 mg QID |
| D | Placebo QID | Placebo QID | Placebo QID | Placebo QID |

The demographics of the four groups was balanced across the groups as shown in Table 4.

TABLE 4

| | Pla- | Oxycodone | Oxycodone Plus | Oxycodone Plus |
|---|---|---|---|---|
| | cebo | QID | Naltrexone QID | Naltrexone BID |
| ITT | 51 | 102 | 104 | 103 |
| Population | | | | |
| Female | 69% | 70% | 69% | 69% |
| Male | 31% | 30% | 31% | 31% |
| Age | 56.0 | 53.5 | 53.6 | 55.1 |
| Weight | 90.7 | 93.3 | 96.0 | 94.6 |
| (kg) | | | | |

All treatment groups were scheduled for QID dosing to protect the double-blind study design as shown in Table 5.

TABLE 5

| Treatment | QID Daily Dosing Scheme* | | | |
|---|---|---|---|---|
| Group | Upon Waking | Noon | Afternoon | Bedtime |
| A | Oxycodone and | Placebo | Oxycodone and | Placebo |
| | Naltrexone | | Naltrexone | |
| B | Oxycodone and | Oxycodone | Oxycodone and | Oxycodone |
| | Naltrexone | and | Naltrexone | and |
| | | Naltrexone | | Naltrexone |
| C | Oxycodone | Oxycodone | Oxycodone | Oxycodone |
| D | Placebo | Placebo | Placebo | Placebo |

*Doses were to be taken 30-60 minutes before meals and at least 4 hours apart. On Day 1 (Week 1 only), patients were to receive three doses of study drug (noon, afternoon and bedtime).

During the 3-week treatment period, patients recorded their PI every 24 hours in their daily diary immediately before their bedtime dose. In addition, patients recorded adverse events and date/time of taking the study medication in the daily diary. Patients returned to the clinic on Week 2, Day 1; Week 3, Day 1 and for End of Treatment assessments (± one day) by the investigator. At each clinic visit, the investigator also collected, additional data, including quality of analgesia, pain control, the SF-12 Health Survey, the WOMAC Osteoarthritis Index and a global assessment of study medication. Patients were required to return for a post-treatment follow-up visit approximately one week after the final dose of study medication (± two days).

Safety was evaluated by vital signs (blood pressure, heart rate, respiratory rate and temperature), physical examinations, EKGs, clinical laboratory tests, adverse events, opioid toxicity assessments and the assessment of opiate withdrawal symptoms using the Short Opiate Withdrawal Scale (SOWS) as shown in Table 6 below.

TABLE 6
Short Opiate Withdrawal Scale
Please put a check mark in the appropriate box for
each of the following conditions in the last 24 hours:

| Description | None | Mild | Moderate | Severe |
|---|---|---|---|---|
| Feeling Sick | | | | |
| Stomach Cramps | | | | |
| Muscle Spasms/Twitching | | | | |
| Feelings of Coldness | | | | |
| Heart Pounding | | | | |
| Muscular Tension | | | | |
| Aches and Pains | | | | |
| Yawning | | | | |
| Runny Eyes | | | | |
| Insomnia/Problems Sleeping | | | | |

Note:
This table shows the 10 items of SOWS and the format in which it is administered.

The Study Population was three hundred sixty-two (360) patients with moderate to severe chronic pain due to osteoarthritis of the hip or knee. According to the study design described above, there were to be about 100 patients each in the oxycodone and naltrexone BID, oxycodone and naltrexone QID and oxycodone alone treatment groups; and about 50 patients in the placebo group.

Inclusion criteria were as follows:
(1) Males and females who were >18 and <70 years of age;
(2) Females who were postmenopausal, physically incapable of childbearing, or practicing an acceptable method of birth control. Acceptable methods of birth control included surgical sterilization, hormonal contraceptives, or double-barrier methods (condom or diaphragm with a spermicidal agent or IUD). If practicing an acceptable method of birth control, a negative urine pregnancy test result was obtained at the Screening Visit;
(3) Patient was ambulatory;
(4) Patient had moderate to severe pain in one or more hip or knee joint(s) caused by osteoarthritis for at least three months prior to the Screening Visit;
(5) Patient had moderate to severe pain in the hip or knee joint(s) while taking one or more oral analgesic medication(s) (e.g., NSAIDs, COX-2 inhibitors, tramadol, opioid) in the past one month prior to the Screening Visit;
(6) Patient had a pain intensity score of >5 on an 11-point numerical scale at the Screening Visit;
(7) Patient had a mean daily diary overall pain intensity (taken immediately before their bedtime dose of acetaminophen) of >5 on an 11-point numerical scale during the last two days of the 4- to 7-day washout period and a confirmatory pain intensity level of >5 on an 11-point numerical scale measured at the clinic at Visit 2;
(8) Patient was able to understand and cooperate with study procedures, and has signed a written informed consent prior to entering the study; and
(9) Patient agreed to refrain from taking any pain medications other than study drug during the 3-week treatment period. (Aspirin (up to 325 mg/day) was permitted for cardiovascular prophylaxis if at a stable dose one month prior to the Screening Visit.)

Exclusion criteria for subjects were as follows:
(1) Patient had received a daily opioid dose equivalent (if applicable) of oxycodone>20 mg for two or more days within four weeks prior to the Screening Visit (as calculated by the Drug Conversion Calculator Version 2.0, American Pain Study);
(2) Patient had received an opioid within 72 hours of the Screening Visit;
(3) Patient weighed more than 300 lbs or less than 100 lbs;
(4) Patient had major surgery within three months prior to the Screening Visit or had surgery planned for this joint during the proposed study period;
(5) Patient had received oral or parenteral corticosteroid therapy within one month prior to the Screening Visit;
(6) Patient had received an intraarticular injection of hyaluronic acid within nine months prior to the Screening Visit;
(7) Patient had received any epidural or intrathecal infusion of any analgesic medication(s) within one month prior to the Screening Visit;
(8) Patient was pregnant or breast-feeding;
(9) Patient had a history of severe hepatic or renal impairment;
(10) Patient had acute hepatitis;
(11) Patient had a known allergy or significant reaction to any of the study medications;
(12) Patient had severe impairment of pulmonary function, hypercarbia, hypoxia, significant chronic obstructive airways disease or cor pulmonale, acute or severe bronchial asthma, sleep apnea syndrome or respiratory depression;
(13) Patient had or is suspected of having paralytic ileus, acute abdomen (serious abdominal pain requiring emergency surgery), or delayed gastric emptying;
(14) Patient had chronic biliary tract disease, chronic pancreatitis, or inflammatory bowel disorders;
(15) Patient had untreated myxedema, untreated hypothyroidism, elevated intracranial pressure, severe anemia, adrenocortical insufficiency, hypotension or hypovolemia;
(16) Patient had other diseases significant enough, in the opinion of the Investigator, to pose a risk for the administration of study drug or that will interfere with pain assessments;
(17) Patient had started or stopped monoamine oxidase inhibitors, tricyclic antidepressant drugs, serotonin reuptake inhibitors, glucosamine/chondroitin, or St. John's Wort within four weeks prior to receiving study medication. A constant dose for longer than four weeks is acceptable;
(18) Patient was receiving high doses of sedatives, hypnotics or tranquilizers;
(19) Patient was receiving phenothiazines or other agents that compromise vasomotor tone;
(20) Patient had a history of alcohol or drug abuse;
(21) Patient had previously received the investigational study drug of oxycodone and naltrexone;
(22) Patient had participated in another investigational drug trial or therapeutic trial within 30 days of the Screening Visit; or
(23) Patient had taken analgesic medication (other than acetaminophen—up to 5 caplets per day) during the 4- to 7-day washout period prior to randomization.

The physical descriptions of the drugs used for the study were as follows. For the 4- to 7-day washout period, a container of acetaminophen (500 mg caplets) was dispensed at the Screening Visit in a sufficient quantity for dosing up to five caplets per day. The investigational drug supplies were in tablet dosage forms containing oxycodone HCl and naltrexone HCl, oxycodone HCl or placebo. All of the tablet dosage forms were indistinguishable from one another to facilitate blinding. The tablets were round (approximately 7 mm diameter), biconvex and had a pale yellow color coating. The investigational drug supplies were dispensed in these weekly kits.

The study procedures were as follows. Prior to any study-related activities, written informed consent was signed and dated by the patient. Clinical examinations were performed that comprised the standard-of-care evaluations routinely performed as part of ongoing care for patients with moderate to severe chronic pain due to osteoarthritis of the hip or knee. Pain assessments were performed by assessing: (1) Pain Intensity, (2) Quality of Analgesia, (3) Pain Control, and (4) Global Assessment of Study Medication.

Pain Intensity was assessed by prompting the patient with the question, “How would you rate your overall pain intensity at this time?”, and the PI score was recorded in the clinic. Pain Intensity was also assessed by prompting the patient with the question, “How would you rate your overall pain intensity during the past 24 hours?”, and a daily PI diary score was recorded by the patient at bedtime. For both Pain Intensity prompts, the response was scored on an 11-point numerical scale (0=no pain and 10=severe pain).

Quality of Analgesia was assessed weekly at clinic visits. The patient was prompted with the question, “How would you rate the quality of your pain relief at this time?”, and responses were selected from poor, fair, good, very good, and excellent.

Pain Control was also assessed weekly at clinic visits. The patient was prompted with the question, “During the past week, how would you describe your pain control during the course of each day?” Responses were selected from: Pain was controlled for (1) a few hours or less each day; (2) several hours each day; (3) most of each day; and (4) throughout each day.

Global Assessment of Study Medication was also assessed weekly at clinic visits. The patient was prompted with the question, “How would you rate the study medication you received this past week? (Please consider the quality of your pain relief, your side effects, your activity level, your mood and sense of well-being, etc. in this evaluation.)”. Responses were selected from poor, fair, good, very good, and excellent.

Additionally, functional assessments were conducted with the SF-12 Health Survey (see Table 1) and the WOMAC Osteoarthritis Index (see Table 2).

Safety procedures included vital signs (blood pressure, respiratory rate, heart rate and temperature), physical examinations, EKGs, clinical laboratory tests, adverse events, opioid toxicity assessments and the assessment of opiate withdrawal symptoms using the SOWS (see Table 6). The opioid toxicity assessment included: (A) CNS review by assessing for (1) confusion, altered mental state, (2) excessive drowsiness, lethargy, stupor, (3) slurred speech (new onset), (4) respiratory, (5) hypoventilation, shortness of breath, apnea, (6) hypoxia, hypercarbia; and (b) cardiac review by assessing for bradycardia, hypotension, and shock. If patients experienced any of these or other symptoms that, in the principal investigator's opinion, would pose a significant risk if additional opioid doses were administered, doses were not escalated on Week 2, Day 1 or Week 3, Day 1.

At the first visit, pre-enrollment screening was performed. The following assessments were conducted at Visit 1, four to seven days prior to enrollment in the study: (1) written informed consent, (2) clinic PI, (3) review inclusion and exclusion criteria, (4) detailed medical history including concomitant medications taken one month prior to the screening visit, (5) complete physical examination including height, weight and vital signs, (6) EKG (QTc interval only), (7) blood samples for clinical laboratory tests (hematology and chemistry), (8) urine sample for clinical laboratory tests, (drug screening and urinalysis), (9) urine pregnancy test for all women of childbearing potential, and (10) dispense acetaminophen, take-home diary and provide an appointment card for the next visit. The study nurse thoroughly reviewed each section of the diary with the patient. The diary issued at Visit 1 was to be used by the patient to record the following information at bedtime immediately before the patient's dose of acetaminophen was taken: (a) overall PI in the past 24 hours, (b) signs and symptoms, and (c) date/time of each acetaminophen dose.

The second visit was on the first day of the first treatment week of the study. The patients returned to the study center four to seven days after the Screening Visit for completion of the pre-dose assessments. This visit included (1) reviewing the take-home diary from the past four to seven days; (2) collecting the bottle of acetaminophen and performing accountability; (3) a baseline clinic PI rating, (4) reviewing inclusion and exclusion criteria. This assessment also included verifying that (a) the mean daily overall pain intensity score collected in the diary over the last two days of the 4- to 7-day washout period was ≧5 (on a scale of 0 to 10) while off all analgesic medications (except acetaminophen as directed); (b) the clinic PI at this visit measured≧5 (on a scale of 0 to 10); and (c) checking that the clinical laboratory tests results from the screening visit were without significant clinical abnormalities, that the urine pregnancy test was negative (if required), and that the urine drug screen was negative.

Patients meeting the study entry criteria were randomly assigned to one of the four treatment groups, and were assigned a randomization number and study medication kit number. The following assessments were then conducted: (1) a brief (interim) medical history; (2) vital signs; (3) blood sample for PK analysis (procedures for collection, storage and shipping of PK samples were provided under separate cover); (4) review and record concomitant medications; (5) SF-12 Health Survey; and (6) WOMAC Osteoarthritis Index.

Once these assessments and procedures were completed, the study medication kit was dispensed for Week 1 (Study Days 1-8). Patients were instructed to take up to three doses of study medication on this day (noon, afternoon and at bedtime). In addition, patients were instructed to take their Day 8 ‘waking’ dose from this medication kit. The patients received their take-home daily diaries and were provided with an appointment card for the next visit. The study nurse thoroughly reviewed each section of the diary with the patient. The daily diary issued at Visit 2 was used to record the following information at Bedtime immediately prior to dosing: (1) overall PI in the past 24 hours; (2) Date and time of each dose of study medication taken; and (3) adverse events.

Patients were contacted by telephone on the evenings of Days 3 and 4 of Treatment Week 1. On Day 3, patients were contacted to determine whether the dose should be escalated on the morning of Day 4. On Day 4, patients were contacted to determine whether patients were tolerating the higher dose. The telephone visits were also used to check for adverse events, compliance and concomitant medications and to remind the patients to complete the daily diary and bring it to the next visit.

Patients returned to the study center for their third visit on Week 2, Day 1 (±one day) for the following:
(1) opioid toxicity assessment;
(2) review take-home diary;
(3) record new/changed adverse events and concomitant medications;
(4) collect study medication from Week 1 (Study Days 1-8) and account for used/unused supplies;
(5) vital signs;
(6) blood sample for PK analysis;
(7) quality of analgesia;
(8) pain control;
(9) global assessment of study medication;
(10) SF-12 Health Survey;
(11) WOMAC Osteoarthritis Index; and
(12) dispense take-home daily diary and study medication kit for Week 2 (Study Days 1-8).

Patients were instructed to take up to three doses of study medication on this day (noon, afternoon and at bedtime). In addition, patients were instructed to take their Day 8 ‘waking’ dose from this medication kit. At the conclusion of this visit, the patient was given an appointment card for the next study visit.

Patients were contacted by telephone on the evening of Day 1 of Treatment Week 2 to determine whether they are tolerating the higher dose; to check for adverse events, compliance and concomitant medications; and to remind them to complete the daily diary and bring it to the next visit.

Patients returned to the study center for their fourth visit on Week 3, Day 1 (±one day) for the following:
(1) opioid toxicity assessment;
(2) review take-home diary;
(3) record new/changed adverse events and concomitant medications;
(4) collect study medication from Week 2 (Study Days 1-8) and account for used/unused supplies;
(5) vital signs;
(6) blood sample for pk analysis;
(7) quality of analgesia;
(8) pain control;
(9) global assessment of study medication;
(10) SF-12 Health Survey;
(11) WOMAC Osteoarthritis Index; and
(12) dispense take-home daily diary and study medication for Week 3 (Study Days 1-8).

Patients were instructed to take up to three doses of study medication on this day (noon, afternoon and at bedtime). In addition, patients were instructed to take their Day 8 ‘waking’ dose from this medication kit. At the conclusion of this visit, the patient was given an appointment card for the next study visit.

Patients were contacted by telephone on the evening of Day 1 of Treatment Week 3 to determine whether they are tolerating the higher dose; to check for adverse events, compliance and concomitant medications; and to remind them to complete the daily diary and bring it to the next visit.

Patients returned to the study center for their fifth (End of Treatment) visit on Week 3, Day 8 (±one day) for the following:
(1) review take-home diary;
(2) record new/changed adverse events and concomitant medications;
(3) collect study medication from Week 3 (Study Days 1-8) and account for used/unused supplies;
(4) complete physical examination and vital signs;
(5) EKG (QTc interval only);
(5) blood samples for clinical laboratory tests (hematology and chemistry);
(6) urine sample for clinical laboratory tests (urinalysis);
(7) blood sample for PK analysis;
(8) quality of analgesia;
(9) pain control;
(10) global assessment of study medication;
(11) SF-12 Health Survey;
(12) WOMAC Osteoarthritis Index;
(13) SOWS; and
(14) dispense take-home daily diary (SOWS and adverse event log) for follow-up period.

Blood samples that were taken during the study at various patient visits were used for a variety of analyses including clinical laboratory tests, PK analysis (see, e.g., Example 3) and cytokine analysis (see, e.g., Example 4).

At the conclusion of this visit, prior to departing the center, the patient was given an appointment card for the next study visit. Patients were instructed to contact the study center immediately if they experienced severe signs and symptoms of opioid withdrawal.

The study center contacted patients before noon once daily (for four days after the last dose of study medication) to monitor for symptoms of opioid withdrawal. On each telephone call, the study center verified that the SOWS have been completed each day (in the morning) by the patients. In addition, there was a check for adverse events and concomitant medications. If necessary, a clinic visit was required for those patients with clinically significant withdrawal symptoms.

Patients returned to the study center approximately one week (±two days) after the last dose of study medication for a post-treatment follow-up visit. At this visit, the following assessments were completed:
(1) review take-home diary; and
(2) record new/changed adverse events and concomitant medications.

Patients could choose to discontinue study drug or study participation at any time, for any reason, specified or unspecified, and without prejudice. If a patient chose to discontinue study drug early, the investigator requested that the patient return to the clinic within 24 hours of stopping the study medication and complete the end-of-treatment assessments described above, as well as the opioid withdrawal monitoring period described above. The investigator also requested that the patient remain in the study for the post-treatment follow-up visit.

For randomization and blinding of the study, the randomization was stratified on patient sex; it was not stratified on investigator. The randomization schedule was generated using a permuted blocks algorithm. The study randomization was unblinded only after all study patients completed therapy and the database was finalized and locked.

The primary analysis population for both efficacy and safety included all patients who took study medication. In the event that a patient was randomized incorrectly or was otherwise administered the incorrect study drug, the patient was to be analyzed according to the study drug actually received.

For the efficacy analysis, endpoints were represented and analyzed by week. Missing efficacy data was imputed across weeks using the last-observation-carried-forward (LOCF) method. Thus, the primary procedure for the analysis of efficacy data was based on a LOCF approach.

The daily pain intensity ratings were summarized as follows. For each week, the pain intensity recorded on the last two full days of dosing within the week, restricted to Day 5 or later, was averaged. If only a single observation was available, it was used; otherwise, the endpoint was not defined. The pain intensity averages were represented as both (1) a change from baseline and (2) a percent change from baseline. The baseline value was defined as the average pain intensity over the last two values recorded during the baseline period; if necessary, a single value was used.

The global assessment, quality of analgesia, and pain control, recorded at the end of each week, were summarized in terms of category proportions.

The SF-12 evaluations, recorded at baseline and at the end of each week, were scored as described in Ware et al., “SF-12: How to score the SF-12 physical and mental health summary scales.” QualityMetric Inc., Lincoln, R.I., and the Health Assessment Lab, Boston, Mass. (3d Ed. 1998), which is incorporated by reference herein. The summarization and analysis of the WOMAC Osteoarthritis Index were specified in the Statistical Analysis Plan per the WOMAC User Guide, which is obtainable at the WOMAC organization website www.womac.org/contact/index.cfm and incorporated by reference herein.

For primary analysis of data, the primary efficacy endpoint was the percent change from baseline in pain intensity at Week 3. Percent change in pain intensity was analyzed using ANOVA methods. The ANOVA model included factors for treatment, sex, and their interaction. Additional covariates could be added to the model for exploratory purposes. Pairwise treatment group comparisons were made using contrasts within the ANOVA framework. Testing employed Type III sums of squares. If the assumptions of the parametric tests were not valid, non-parametric tests were used.

For secondary analysis of data, pain intensity changes, SF-12, and WOMAC were analyzed with the ANOVA methods as described above. Within treatment arms, weeks were compared in pairwise fashion using paired-sample methods. The global assessments, quality of analgesia ratings, and pain control was analyzed at each week, globally and in pairwise fashion, using the Cochran-Mantel-Haenszel row mean scores (CMH-RMS) test, using equally spaced scores. An “observed data” analysis, without any data imputation, was conducted on pain intensity changes, global assessments, quality of analgesia ratings, pain control, SF-12, and WOMAC using the same analysis methods described previously.

Adverse events reported were mapped to preferred terms and organ systems using the MedDRA mapping system. Adverse events were associated with weeks according to their onset date. The number and percentage of patients reporting each event were summarized by treatment group and week.

Treatment groups were examined for differences in the incidence and severity of selected opioid-associated adverse events, including constipation, dizziness, somnolence, headache, pruritus, nausea, vomiting, urinary retention, and bradypnoea. The homogeneity of response between males and females was investigated descriptively.

Each of the SOWS assessments (Gossop, “The Development of a Short Opiate withdrawal Scale (SOWS).” Addictive Behaviors, Vol. 15, p. 487-490, 1990 (incorporated by reference herein)) on Days 1 through 4 of opioid withdrawal monitoring was reduced to an average symptom score and was summarized in terms of changes from baseline, which was defined as the in-clinic assessment at the end of treatment visit (Week 3, Day 8).

The study's sensitivity was broadly assessed by calculating the power of the Wilcoxon test to detect a pairwise treatment difference in an underlying normally distributed endpoint where the two treatment group means differ by one-half a standard deviation. Under these assumption, the statistical power of a 2-sided Wilcoxon test was:

| Analyzable Sample | Power of a Pairwise Treatment Comparison | |
|---|---|---|
| Size per Group | 0.05-level | 0.025-level |
| 100 | 92% | 87% |
| 90 | 89% | 83% |
| 80 | 85% | 78% |
| 70 | 80% | 72% |

Results were obtained using nQuery Advisor®, version 4.0 (Statistical Solutions Ltd., Boston, Mass.).

One efficacy endpoint for this study was percent change in pain intensity from baseline to Week 3. In general, a dose response relationship was observed. That is, greater reductions in means PI occurred as the dose increased in all active treatment groups. The greatest reduction occurred in the oxycodone plus naltrexone BID combination treatment group. The mean percent change in pain intensity from baseline to Week 3 was 39.2% for this BID group. This was both clinically and statistically significant when compared to the other treatment groups. Tables 7A, 7B, and 7C show averages for actual values for Pain Intensity at each of Weeks 1, 2 and 3, based on the Intent to Treat Population using the LOCF method and Table 7D shows the baseline values.

TABLE 7A
PAIN INTENSITY - BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 1 | | | | | |
| N | 46 | 93 | 96 | 84 | 319 |
| MEAN | 6.5 | 6.1 | 6.3 | 5.5 | 6.1 |
| STANDARD DEVIATION | 2.10 | 2.17 | 2.14 | 2.11 | 2.16 |
| MINIMUM | 2.5 | 1.0 | 1.0 | 0.0 | 0.0 |
| MEDIAN | 6.5 | 6.0 | 6.8 | 5.5 | 6.0 |
| MAXIMUM | 10.0 | 10.0 | 10.0 | 10.0 | 10.0 |
| MODEL P-VALUES² | | | | | |
| TREATMENT | 0.053 | | | | |
| SEX | 0.727 | | | | |
| TREATMENT + SEX | 0.860 | | | | |
| PAIRWISE COMPARISON P-VALUES² | | | | | |
| PLACEBO QID | | 0.225 | 0.456 | 0.013 | |
| OXY QID | | | 0.561 | 0.108 | |
| OXY + NTX QID | | | | 0.030 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 7B
PAIN INTENSITY - BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 2 | | | | | |
| N | 46 | 93 | 96 | 84 | 319 |
| MEAN | 6.2 | 5.8 | 6.0 | 5.0 | 5.7 |
| STANDARD DEVIATION | 2.47 | 2.25 | 2.20 | 2.21 | 2.29 |
| MINIMUM | 1.0 | 0.0 | 1.0 | 0.0 | 0.0 |
| MEDIAN | 7.0 | 6.0 | 6.0 | 5.0 | 6.0 |
| MAXIMUM | 10.0 | 10.0 | 10.0 | 10.0 | 10.0 |
| MODEL P-VALUES² | | | | | |
| TREATMENT | 0.007 | | | | |
| SEX | 0.972 | | | | |
| TREATMENT + SEX | 0.795 | | | | |
| PAIRWISE COMPARISON P-VALUES² | | | | | |
| PLACEBO QID | | 0.148 | 0.463 | 0.002 | |
| OXY QID | | | 0.375 | 0.046 | |
| OXY + NTX QID | | | | 0.004 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 7C
PAIN INTENSITY-BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 3 | | | | | |
| N | 46 | 93 | 96 | 84 | 319 |
| MEAN | 6.1 | 5.6 | 5.7 | 4.5 | 5.4 |
| STANDARD DEVIATION | 2.79 | 2.33 | 2.43 | 2.44 | 2.51 |
| MINIMUM | 0.0 | 0.0 | 0.0 | 0.0 | 0.0 |
| MEDIAN | 7.0 | 5.5 | 6.0 | 4.3 | 5.5 |
| MAXIMUM | 10.0 | 10.0 | 10.0 | 10.0 | 10.0 |
| MODEL P-VALUES² | | | | | |
| TREATMENT | <0.001 | | | | |
| SEX | 0.416 | | | | |
| TREATMENT + SEX | 0.348 | | | | |
| PAIRWISE COMPARISON P-VALUES² | | | | | |
| PLACEBO QID | | 0.092 | 0.193 | <0.001 | |
| OXY QID | | | 0.630 | 0.009 | |
| OXY + NTX QID | | | | 0.002 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 7D
PAIN INTENSITY - BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| BASELINE | | | | | |
| N | 50 | 102 | 102 | 101 | 355 |
| MEAN | 7.7 | 7.4 | 7.7 | 7.6 | 7.6 |
| STANDARD DEVIATION | 1.29 | 1.30 | 1.41 | 1.37 | 1.35 |
| MINIMUM | 5.0 | 5.0 | 5.0 | 4.5 | 4.5 |
| MEDIAN | 8.0 | 7.0 | 8.0 | 7.5 | 7.5 |
| MAXIMUM | 10.0 | 10.0 | 10.0 | 10.0 | 10.0 |
| MODEL P-VALUES² | | | | | |
| TREATMENT | 0.482 | | | | |
| SEX | 0.018 | | | | |
| TREATMENT + SEX | 0.876 | | | | |
| PAIRWISE | | | | | |
| COMPARISON P-VALUES² | | | | | |
| PLACEBO QID | | 0.220 | 0.915 | 0.568 | |
| OXY QID | | | 0.165 | 0.415 | |
| OXY + NTX QID | | | | 0.564 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

Tables 8A, 8B, and 8C for males and 8E, 8F and 8G for females show averages for actual values for Pain Intensity at Weeks 1, 2 and 3, respectively. Tables 8D and 8H show baseline values for males and females, respectively.

TABLE 8A
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| MALE | (N = 15) | (N = 31) | (N = 32) | (N = 32) | (N = 110) |
| ACTUAL VALUE AT WEEK 1 | | | | | |
| N | 14 | 29 | 29 | 25 | 97 |
| MEAN | 6.7 | 5.9 | 6.1 | 5.5 | 6.0 |
| STANDARD DEVIATION | 1.81 | 2.18 | 2.07 | 1.68 | 1.98 |
| MINIMUM | 3.0 | 1.5 | 1.0 | 2.0 | 1.0 |
| MEDIAN | 7.3 | 6.0 | 6.5 | 5.5 | 6.0 |
| MAXIMUM | 9.0 | 10.0 | 10.0 | 8.0 | 10.0 |
| MODEL P-VALUES² | | | | | |
| TREATMENT | 0.325 | | | | |
| PAIRWISE COMPARISON P-VALUES² | | | | | |
| PLACEBO QID | | 0.217 | 0.332 | 0.069 | |
| OXY QID | | | 0.741 | 0.445 | |
| OXY + NTX QID | | | | 0.280 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8B
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| MALE | (N = 15) | (N = 31) | (N = 32) | (N = 32) | (N = 110) |
| ACTUAL VALUE AT WEEK 2 | | | | | |
| N | 14 | 29 | 29 | 25 | 97 |
| MEAN | 6.6 | 5.5 | 6.0 | 4.9 | 5.7 |
| STANDARD DEVIATION | 2.04 | 2.36 | 2.00 | 1.97 | 2.16 |
| MINIMUM | 3.0 | 0.0 | 1.0 | 1.0 | 0.0 |
| MEDIAN | 7.3 | 5.5 | 6.0 | 5.0 | 6.0 |
| MAXIMUM | 9.0 | 10.0 | 10.0 | 9.0 | 10.0 |
| MODEL P-VALUES² | | | | | |
| TREATMENT | 0.084 | | | | |
| PAIRWISE COMPARISON P-VALUES² | | | | | |
| PLACEBO QID | | 0.117 | 0.366 | 0.017 | |
| OXY QID | | | 0.404 | 0.287 | |
| OXY + NTX QID | | | | 0.064 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8C
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| MALE | (N = 15) | (N = 31) | (N = 32) | (N = 32) | (N = 110) |
| ACTUAL VALUE AT WEEK 3 | | | | | |
| N | 14 | 29 | 29 | 25 | 97 |
| MEAN | 6.6 | 5.0 | 5.6 | 4.0 | 5.2 |
| STANDARD DEVIATION | 2.30 | 2.45 | 2.05 | 2.40 | 2.42 |
| MINIMUM | 2.5 | 0.0 | 1.0 | 0.5 | 0.0 |
| MEDIAN | 7.3 | 4.5 | 6.0 | 4.0 | 5.0 |
| MAXIMUM | 9.5 | 10.0 | 10.0 | 8.5 | 10.0 |
| MODEL P-VALUES² | | | | | |
| TREATMENT | 0.007 | | | | |
| PAIRWISE COMPARISON P-VALUES² | | | | | |
| PLACEBO QID | | 0.034 | 0.149 | <0.001 | |
| OXY QID | | | 0.395 | 0.110 | |
| OXY + NTX QID | | | | 0.017 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8D
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| MALE | (N = 15) | (N = 31) | (N = 32) | (N = 32) | (N = 110) |
| BASELINE | | | | | |
| N | 15 | 33 | 32 | 32 | 110 |
| MEAN | 7.4 | 7.2 | 7.3 | 7.3 | 7.3 |
| STANDARD DEVIATION | 1.03 | 1.11 | 1.35 | 1.19 | 1.18 |
| MINIMUM | 5.5 | 5.0 | 5.0 | 5.0 | 5.0 |
| MEDIAN | 7.5 | 7.0 | 7.5 | 7.0 | 7.0 |
| MAXIMUM | 9.0 | 10.0 | 10.0 | 10.0 | 10.0 |
| MODEL P-VALUES² | | | | | |
| TREATMENT | 0.952 | | | | |
| PAIRWISE | | | | | |
| COMPARISON P-VALUES² | | | | | |
| PLACEBO QID | | 0.583 | 0.812 | 0.780 | |
| OXY QID | | | 0.697 | 0.736 | |
| OXY + NTX QID | | | | 0.959 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8E
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| FEMALE | (N = 35) | (N = 71) | (N = 70) | (N = 70) | (N = 246) |
| ACTUAL VALUE AT WEEK 1 | | | | | |
| N | 32 | 64 | 67 | 59 | 222 |
| MEAN | 6.4 | 6.2 | 6.4 | 5.5 | 6.1 |
| STANDARD DEVIATION | 2.24 | 2.18 | 2.18 | 2.28 | 2.23 |
| MINIMUM | 2.5 | 1.0 | 1.0 | 0.0 | 0.0 |
| MEDIAN | 6.5 | 6.3 | 7.0 | 5.5 | 6.0 |
| MAXIMUM | 10.0 | 10.0 | 10.0 | 10.0 | 10.0 |
| MODEL P-VALUES² | | | | | |
| TREATMENT | 0.081 | | | | |
| PAIRWISE COMPARISON P-VALUES² | | | | | |
| PLACEBO QID | | 0.649 | 0.995 | 0.055 | |
| OXY QID | | | 0.567 | 0.073 | |
| OXY + NTX QID | | | | 0.018 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8F
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| FEMALE | (N = 35) | (N = 71) | (N = 70) | (N = 70) | (N = 246) |
| ACTUAL VALUE AT WEEK 2 | | | | | |
| N | 32 | 64 | 67 | 59 | 222 |
| MEAN | 6.1 | 5.9 | 6.0 | 5.0 | 5.7 |
| STANDARD DEVIATION | 2.64 | 2.20 | 2.29 | 2.32 | 2.35 |
| MINIMUM | 1.0 | 0.0 | 1.5 | 0.0 | 0.0 |
| MEDIAN | 6.5 | 6.3 | 6.0 | 5.0 | 5.8 |
| MAXIMUM | 10.0 | 10.0 | 10.0 | 10.0 | 10.0 |
| MODEL P-VALUES² | | | | | |
| TREATMENT | 0.052 | | | | |
| PAIRWISE COMPARISON P-VALUES² | | | | | |
| PLACEBO QID | | 0.699 | 0.960 | 0.039 | |
| OXY QID | | | 0.676 | 0.040 | |
| OXY + NTX QID | | | | 0.013 | |

NOTE:\
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8G
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| FEMALE | (N = 35) | (N = 71) | (N = 70) | (N = 70) | (N = 246) |
| ACTUAL VALUE AT WEEK 3 | | | | | |
| N | 32 | 64 | 67 | 59 | 222 |
| MEAN | 5.9 | 5.9 | 5.7 | 4.8 | 5.5 |
| STANDARD DEVIATION | 2.98 | 2.25 | 2.59 | 2.44 | 2.54 |
| MINIMUM | 0.0 | 0.0 | 0.0 | 0.0 | 0.0 |
| MEDIAN | 6.0 | 6.0 | 6.0 | 5.0 | 5.5 |
| MAXIMUM | 10.0 | 10.0 | 10.0 | 10.0 | 10.0 |
| MODEL P-VALUES² | | | | | |
| TREATMENT | 0.057 | | | | |
| PAIRWISE COMPARISON P-VALUES² | | | | | |
| PLACEBO QID | | 0.977 | 0.769 | 0.044 | |
| OXY QID | | | 0.744 | 0.016 | |
| OXY + NTX QID | | | | 0.034 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8H
PAIN INTENSITY - BY WEEK¹ AND SEX
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| FEMALE | (N = 35) | (N = 71) | (N = 70) | (N = 70) | (N = 246) |
| BASELINE | | | | | |
| N | 35 | 71 | 70 | 69 | 245 |
| MEAN | 7.9 | 7.4 | 7.9 | 7.7 | 7.7 |
| STANDARD DEVIATION | 1.38 | 1.38 | 1.41 | 1.43 | 1.41 |
| MINIMUM | 5.0 | 5.0 | 5.0 | 4.5 | 4.5 |
| MEDIAN | 8.0 | 7.5 | 8.0 | 7.5 | 8.0 |
| MAXIMUM | 10.0 | 10.0 | 10.0 | 10.0 | 10.0 |
| MODEL P-VALUES² | | | | | |
| TREATMENT | 0.248 | | | | |
| PAIRWISE | | | | | |
| COMPARISON P-VALUES² | | | | | |
| PLACEBO QID | | 0.155 | 0.902 | 0.530 | |
| OXY QID | | | 0.058 | 0.332 | |
| OXY + NTX QID | | | | 0.358 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

Tables 9A, 9B and 9C show the percent change from baseline PI scores at Weeks 1, 2 and 3, respectively.

TABLE 9A
PAIN INTENSITY - BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| PERCENT CHANGE FROM | | | | | |
| BASELINE TO WEEK 1 | | | | | |
| N | 46 | 93 | 96 | 83 | 318 |
| MEAN | −16.6 | −18.4 | −17.8 | −26.7 | −20.1 |
| STANDARD DEVIATION | 21.86 | 25.09 | 26.27 | 25.80 | 25.40 |
| MINIMUM | −75.0 | −83.3 | −83.3 | −100.0 | −100.0 |
| MEDIAN | −12.9 | −16.7 | −15.5 | −25.0 | −17.6 |
| MAXIMUM | 33.3 | 50.0 | 66.7 | 16.7 | 66.7 |
| MODEL P-VALUES² | | | | | |
| TREATMENT | 0.142 | | | | |
| SEX | 0.224 | | | | |
| TREATMENT + SEX | 0.751 | | | | |
| PAIRWISE COMPARISON P-VALUES² | | | | | |
| PLACEBO QID | | 0.496 | 0.612 | 0.044 | |
| OXY QID | | | 0.827 | 0.099 | |
| OXY + NTX QID | | | | 0.062 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 9B
PAIN INTENSITY - BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| PERCENT CHANGE FROM | | | | | |
| BASELINE TO WEEK 2 | | | | | |
| N | 46 | 93 | 96 | 83 | 318 |
| MEAN | −20.4 | −22.9 | −21.2 | −33.9 | −24.9 |
| STANDARD DEVIATION | 28.55 | 27.52 | 28.17 | 27.24 | 28.19 |
| MINIMUM | −84.6 | −100.0 | −83.3 | −100.0 | −100.0 |
| MEDIAN | −13.8 | −20.0 | −20.0 | −33.3 | −21.4 |
| MAXIMUM | 58.3 | 42.9 | 66.7 | 12.5 | 66.7 |
| MODEL P-VALUES² | | | | | |
| TREATMENT | 0.013 | | | | |
| SEX | 0.232 | | | | |
| TREATMENT + SEX | 0.622 | | | | |
| PAIRWISE COMPARISON P-VALUES² | | | | | |
| PLACEBO QID | | 0.341 | 0.718 | 0.007 | |
| OXY QID | | | 0.462 | 0.031 | |
| OXY + NTX QID | | | | 0.004 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 9C
PAIN INTENSITY - BY WEEK¹
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| PERCENT CHANGE FROM | | | | | |
| BASELINE TO WEEK 3 | | | | | |
| N | 46 | 93 | 96 | 83 | 318 |
| MEAN | −21.5 | −24.6 | −26.0 | −39.2 | −28.4 |
| STANDARD DEVIATION | 31.66 | 29.22 | 31.16 | 32.51 | 31.89 |
| MINIMUM | −100.0 | −100.0 | −100.0 | −100.0 | −100.0 |
| MEDIAN | −17.6 | −20.0 | −23.5 | −40.0 | −25.0 |
| MAXIMUM | 58.3 | 42.9 | 66.7 | 55.6 | 66.7 |
| MODEL P-VALUES² | | | | | |
| TREATMENT | 0.002 | | | | |
| SEX | 0.855 | | | | |
| TREATMENT + SEX | 0.221 | | | | |
| PAIRWISE COMPARISON P-VALUES² | | | | | |
| PLACEBO QID | | 0.219 | 0.282 | <0.001 | |
| OXY QID | | | 0.848 | 0.006 | |
| OXY + NTX QID | | | | 0.003 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

Another efficacy endpoint for this study was assessments of quality of analgesia and the results are shown in Tables 10A, 10B and 10C for Weeks 1, 2 and 3, respectively. The oxycodone plus naltrexone BID treatment group show a consistent and greater improvement in the quality of analgesia at each of Weeks 1 and 2 (see Tables 10A and 10B). At Week 3, oxycodone plus naltrexone QID and oxycodone plus naltrexone BID were significantly better than placebo as shown in Table 10C. A quality of analgesia assessment at Week 3 of very good or excellent was reported by 12.0% of patients treated with placebo, 19.6% of patients treated with oxycodone alone QID, 10.6% of patients with oxycodone plus naltrexone QID, and 33.3% of patients treated with oxycodone plus naltrexone BID.

TABLE 10A
QUALITY OF ANALGESIA - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 1 | | | | | | | | | | |
| EXCELLENT (4) | 1 | (2.0%) | 3 | (2.9%) | 3 | (2.9%) | 3 | (2.9%) | 10 | (2.8%) |
| VERY GOOD (3) | 5 | (10.0%) | 8 | (7.8%) | 13 | (12.7%) | 14 | (13.7%) | 40 | (11.2%) |
| GOOD (2) | 13 | (26.0%) | 18 | (37.3%) | 26 | (25.5%) | 29 | (28.4%) | 106 | (29.8%) |
| FAIR (1) | 13 | (26.0%) | 31 | (30.4%) | 33 | (32.4%) | 34 | (33.3%) | 111 | (31.2%) |
| POOR (0) | 17 | (34.0%) | 18 | (17.6%) | 25 | (24.5%) | 21 | (20.6%) | 81 | (22.8%) |
| MISSING | 1 | (2.0%) | 4 | (3.9%) | 2 | (2.0%) | 1 | (1.0%) | 8 | (2.2%) |
| OVERALL P-VALUE¹ | 0.446 | | | | | | | | | |
| PAIRWISE | | | | | | | | | | |
| COMPARISON P-VALUES² | | | | | | | | | | |
| PLACEBO QID | | | 0.125 | | 0.353 | | 0.162 | | | |
| OXY QID | | | | | 0.501 | | 0.925 | | | |
| OXY + NTX QID | | | | | | | 0.572 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 10B
QUALITY OF ANALGESIA - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 2 | | | | | | | | | | |
| EXCELLENT (4) | 2 | (4.0%) | 4 | (3.9%) | 4 | (3.9%) | 6 | (5.9%) | 16 | (4.5%) |
| VERY GOOD (3) | 8 | (16.0%) | 16 | (15.7%) | 13 | (12.7%) | 28 | (27.5%) | 65 | (18.3%) |
| GOOD (2) | 15 | (30.0%) | 30 | (29.4%) | 37 | (36.3%) | 21 | (20.6%) | 103 | (28.9%) |
| FAIR (1) | 6 | (12.0%) | 29 | (28.4%) | 24 | (23.5%) | 28 | (27.5%) | 87 | (24.4%) |
| POOR (0) | 18 | (36.0%) | 19 | (18.6%) | 22 | (21.6%) | 18 | (17.6%) | 77 | (21.6%) |
| MISSING | 1 | (2.0%) | 4 | (3.9%) | 2 | (2.0%) | 1 | (1.0%) | 8 | (2.2%) |
| OVERALL P-VALUE¹ | 0.254 | | | | | | | | | |
| PAIRWISE | | | | | | | | | | |
| COMPARISON P-VALUES² | | | | | | | | | | |
| PLACEBO QID | | | 0.391 | | 0.478 | | 0.081 | | | |
| OXY QID | | | | | 0.841 | | 0.221 | | | |
| OXY + NTX QID | | | | | | | 0.155 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 10C
QUALITY OF ANALGESIA - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 3 | | | | | | | | | | |
| EXCELLENT (4) | 0 | (0.0%) | 5 | (4.9%) | 3 | (2.9%) | 8 | (7.8%) | 16 | (4.5%) |
| VERY GOOD (3) | 6 | (12.0%) | 15 | (14.7%) | 17 | (16.7%) | 26 | (25.5%) | 64 | (18.0%) |
| GOOD (2) | 14 | (28.0%) | 32 | (31.4%) | 34 | (33.3%) | 22 | (21.6%) | 102 | (28.7%) |
| FAIR (1) | 11 | (22.0%) | 23 | (22.5%) | 27 | (26.5%) | 28 | (27.5%) | 89 | (25.0%) |
| POOR (0) | 18 | (36.0%) | 23 | (22.5%) | 19 | (18.6%) | 17 | (16.7%) | 77 | (21.6%) |
| MISSING | 1 | (2.0%) | 4 | (3.9%) | 2 | (2.0%) | 1 | (1.0%) | 8 | (2.2%) |
| OVERALL P-VALUE¹ | 0.017 | | | | | | | | | |
| PAIRWISE | | | | | | | | | | |
| COMPARISON P-VALUES² | | | | | | | | | | |
| PLACEBO QID | | | 0.052 | | 0.028 | | 0.002 | | | |
| OXY QID | | | | | 0.855 | | 0.139 | | | |
| OXY + NTX QID | | | | | | | 0.173 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

Another efficacy endpoint for this study was a global assessment and the results are shown in Tables 11A, 11B and 11C for Weeks 1, 2 and 3, respectively. At Week 3, oxycodone plus naltrexone QID and oxycodone plus naltrexone BID were statistically significantly better than placebo (in pairwise comparisons) as shown in Table 1° C. A global assessment of very good or excellent at Week 3 was reported by 16.0% of patients treated with oxycodone plus naltrexone QID, 19.6% of patients treated with oxycodone alone QID, 22.5% of patients with oxycodone plus naltrexone QID, and 30.4% of patients treated with oxycodone plus naltrexone BID. Tables 11A, 11B and 11C also show the p value vs. placebo calculated for the scores from the global assessment for Weeks 1, 2 and 3, respectively, which were determined using the Cochran-Mantel-Haenszel row mean scores (CMH-RMS) test, using equally spaced scores. Thus, the results in Table 11C generally show a population shift from patient responses of poor and fair toward patient responses of very good and excellent when comparing the placebo group to the oxycodone alone QID, oxycodone plus naltrexone QID and oxycodone plus naltrexone BID treatment groups. Larger percentages of patients in the oxycodone plus naltrexone BID treatment group gave responses of very good or excellent.

TABLE 11A
GLOBAL ASSESSMENT - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 002) | | (N = 102) | | (N = 356) | |
| WEEK 1 | | | | | | | | | | |
| EXCELLENT (4) | 0 | (0.0%) | 3 | (2.9%) | 5 | (4.9%) | 2 | (2.0%) | 10 | (2.8%) |
| VERY GOOD (3) | 8 | (16.0%) | 12 | (11.8%) | 17 | (16.7%) | 18 | (17.6%) | 55 | (15.4%) |
| GOOD (2) | 11 | (22.0%) | 30 | (29.4%) | 27 | (26.5%) | 31 | (30.4%) | 99 | (27.8%) |
| FAIR (1) | 14 | (28.0%) | 37 | (36.3%) | 25 | (24.5%) | 26 | (25.5%) | 102 | (28.7%) |
| POOR (0) | 16 | (32.0%) | 16 | (15.7%) | 26 | (25.5%) | 24 | (23.5%) | 82 | (23.0%) |
| MISSING | 1 | (2.0%) | 4 | (3.9%) | 2 | (2.0%) | 1 | (1.0%) | 8 | (2.2%) |
| OVERALL P-VALUE¹ | 0.487 | | | | | | | | | |
| PAIRWISE | | | | | | | | | | |
| COMPARISON P-VALUES² | | | | | | | | | | |
| PLACEBO QID | | | 0.160 | | 0.174 | | 0.173 | | | |
| OXY QID | | | | | 0.896 | | 0.970 | | | |
| OXY + NTX QID | | | | | | | 0.927 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 11B
GLOBAL ASSESSMENT - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 2 | | | | | | | | | | |
| EXCELLENT (4) | 0 | (0.0%) | 6 | (5.9%) | 4 | (3.9%) | 6 | (5.9%) | 16 | (4.5%) |
| VERY GOOD (3) | 10 | (20.0%) | 14 | (33.7%) | 14 | (13.7%) | 20 | (19.6%) | 58 | (16.3%) |
| GOOD (2) | 13 | (26.0%) | 13 | (32.4%) | 38 | (37.3%) | 28 | (27.5%) | 112 | (31.5%) |
| FAIR(l) | 7 | (14.0%) | 29 | (28.4%) | 22 | (21.6%) | 23 | (22.5%) | 81 | (22.8%) |
| POOR (0) | 19 | (38.0%) | 16 | (15.7%) | 22 | (21.6%) | 24 | (23.5%) | 81 | (22.8%) |
| MISSING | 1 | (2.0%) | 4 | (3.9%) | 2 | (2.0%) | 1 | (1.0%) | 9 | (2.2%) |
| OVERALL P-VALUE¹ | 0.319 | | | | | | | | | |
| PAIRWISE | | | | | | | | | | |
| COMPARISON P-VALUES² | | | | | | | | | | |
| PLACEBO QID | | | 0.074 | | 0.166 | | 0.120 | | | |
| OXY QID | | | | | 0.597 | | 0.860 | | | |
| OXY + NTX QID | | | | | | | 0.742 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 11C
GLOBAL ASSESSMENT - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 3 | | | | | | | | | | |
| EXCELLENT (4) | 1 | (2.0%) | 6 | (5.9%) | 5 | (4.9%) | 9 | (8.8%) | 21 | (5.9%) |
| VERY GOOD (3) | 7 | (14.0%) | 14 | (13.7%) | 18 | (17.6%) | 22 | (21.6%) | 61 | (17.1%) |
| GOOD (2) | 13 | (26.0%) | 36 | (35.3%) | 34 | (33.3%) | 23 | (22.5%) | 106 | (29.8%) |
| FAIR (1) | 9 | (18.0%) | 18 | (17.6%) | 21 | (20.6%) | 22 | (21.6%) | 70 | (19.7%) |
| POOR (0) | 19 | (38.0%) | 24 | (23.5%) | 22 | (21.6%) | 25 | (24.5%) | 90 | (25.3%) |
| MISSING | 1 | (2.0%) | 4 | (3.9%) | 2 | (2.0%) | 1 | (1.0%) | 8 | (2.2%) |
| OVERALL P-VALUE¹ | 0.165 | | | | | | | | | |
| PAIRWISE | | | | | | | | | | |
| COMPARISON P-VALUES² | | | | | | | | | | |
| PLACEBO QID | | | 0.078 | | 0.048 | | 0.039 | | | |
| OXY QID | | | | | 0.818 | | 0.604 | | | |
| OXY + NTX QID | | | | | | | 0.760 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

Another efficacy endpoint for this study was an assessment of pain control and the results are shown in Tables 12A, 12B and 12C for Weeks 1, 2 and 3, respectively. From Week 1 to Weeks 2 and 3, there were significant changes within treatment group (indicating better control throughout the day) in the pain control assessments for the oxycodone plus naltrexone BID treatment group. There were also statistically significant changes for oxycodone plus naltrexone QID from Week 1 to Week 3 and for placebo from Week 1 to Week 3. As shown in Tables 12A, 12B and 12C, patients in the oxycodone plus naltrexone BID treatment group showed improved pain control when compared to placebo at each week of treatment. Tables 12A, 12B and 12C also show the p value vs. placebo calculated for the scores from Pain Control, which were determined using the Cochran-Mantel-Haenszel row mean scores (CMH-RMS) test, using equally spaced scores.

TABLE 12A
PAIN CONTROL - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 1 | | | | | | | | | | |
| PAIN CONTROLLED | 2 | (4.0%) | 10 | (9.8%) | 5 | (4.9%) | 5 | (4.9%) | 22 | (6.2%) |
| THROUGHOUT EACH DAY (4) | | | | | | | | | | |
| PAIN CONTROLLED | 8 | (16.0%) | 17 | (16.7%) | 20 | (19.6%) | 23 | (22.5%) | 68 | (19.1%) |
| MOST OF EACH DAY (3) | | | | | | | | | | |
| PAIN CONTROLLED | 8 | (16.0%) | 29 | (28.4%) | 31 | (30.4%) | 29 | (28.4%) | 97 | (27.2%) |
| SEVERAL HRS EACH DAY (2) | | | | | | | | | | |
| PAIN CONTROLLED A FEW | 31 | (62.0%) | 42 | (41.2%) | 44 | (43.1%) | 44 | (43.1%) | 161 | (45.2%) |
| HRS/LESS EACH DAY (1) | | | | | | | | | | |
| MISSING | 1 | (2.0%) | 4 | (3.9%) | 2 | (2.0%) | 1 | (1.0%) | 8 | (2.2%) |
| OVERALL P-VALUE¹ | | 0.227 | | | | | | | | |
| PAIRWISE | | | | | | | | | | |
| COMPARISON P-VALUES² | | | | | | | | | | |
| PLACEBO QID | | | 0.051 | | 0.120 | | 0.084 | | | |
| OXY QID | | | | | 0.514 | | 0.673 | | | |
| OXY + NTX QID | | | | | | | 0.810 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 12B
PAIN CONTROL - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 2 | | | | | | | | | | |
| PAIN CONTROLLED | 2 | (4.0%) | 10 | (9.8%) | 6 | (5.9%) | 9 | (8.8%) | 27 | (7.6%) |
| THROUGHOUT EACH DAY (4) | | | | | | | | | | |
| PAIN CONTROLLED | 11 | (22.0%) | 26 | (25.5%) | 29 | (28.4%) | 33 | (32.4%) | 99 | (27.8%) |
| MOST OF EACH DAY (3) | | | | | | | | | | |
| PAIN CONTROLLED | 11 | (22.0%) | 26 | (25.5%) | 26 | (25.5%) | 18 | (17.6%) | 81 | (22.8%) |
| SEVERAL HRS EACH DAY (2) | | | | | | | | | | |
| PAIN CONTROLLED A FEW | 25 | (50.0%) | 36 | (35.3%) | 39 | (38.2%) | 41 | (40.2%) | 141 | (39.6%) |
| HRS/LESS EACH DAY (1) | | | | | | | | | | |
| MISSING | 1 | (2.0%) | 4 | (3.9%) | 2 | (2.0%) | 1 | (1.0%) | 8 | (2.2%) |
| OVERALL P-VALUE¹ | 0.299 | | | | | | | | | |
| PAIRWISE | | | | | | | | | | |
| COMPARISON P-VALUES² | | | | | | | | | | |
| PLACEBO QID | | | 0.080 | | 0.179 | | 0.087 | | | |
| OXY QID | | | | | 0.560 | | 0.983 | | | |
| OXY + NTX QID | | | | | | | 0.577 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 12C
PAIN CONTROL - BY WEEK
ANALYSIS POPULATION: INTENT TO TREAT POPULATION

| | PLACEBO | | OXY | | OXY + NTX | | OXY + NTX | | | |
|---|---|---|---|---|---|---|---|---|---|---|
| | QID | | QID | | QID | | BID | | TOTAL | |
| | (N = 50) | | (N = 102) | | (N = 102) | | (N = 102) | | (N = 356) | |
| WEEK 3 | | | | | | | | | | |
| PAIN CONTROLLED | 3 | (6.0%) | 8 | (7.8%) | 7 | (6.9%) | 12 | (21.8%) | 30 | (8.4%) |
| THROUGHOUT EACH DAY (4) | | | | | | | | | | |
| PAIN CONTROLLED | 10 | (20.0%) | 27 | (26.5%) | 31 | (30.4%) | 32 | (31.4%) | 100 | (28.1%) |
| MOST OF EACH DAY (3) | | | | | | | | | | |
| PAIN CONTROLLED | 11 | (22.0%) | 28 | (27.5%) | 22 | (21.6%) | 16 | (17.6%) | 79 | (22.2%) |
| SEVERAL HRS EACH DAY (2) | | | | | | | | | | |
| PAIN CONTROLLED A FEW | 25 | (50.0%) | 35 | (34.3%) | 40 | (39.2%) | 39 | (38.2%) | 139 | (39.0%) |
| HRS/LESS EACH DAY (1) | | | | | | | | | | |
| MISSING | 1 | (2.0%) | 4 | (3.9%) | 2 | (2.0%) | 1 | (1.0%) | 8 | (2.2%) |
| OVERALL P-VALUE¹ | 0.259 | | | | | | | | | |
| PAIRWISE | | | | | | | | | | |
| COMPARISON P-VALUES² | | | | | | | | | | |
| PLACEBO QID | | | 0.123 | | 0.177 | | 0.055 | | | |
| OXY QID | | | | | 0.822 | | 0.553 | | | |
| OXY + NTX QID | | | | | | | 0.419 | | | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

Another efficacy endpoint for this study was a functional assessment using WOMAC, including its three subscales for pain, stiffness and physical function. The actual values from the WOMAC pain subscale are shown in Tables 13A, 13B and 13C for Weeks 1, 2 and 3, respectively and Table 13D shows the baseline values. Greater improvements (% change from baseline to Week 3) were observed with BID administration of oxycodone plus naltrexone in all categories (pain, stiffness, or physical function). Oxycodone plus naltrexone BID was statistically significantly better than placebo at Weeks 2 and 3 as measured by the WOMAC pain subscale, stiffness subscale, physical function subscale and total score, as shown below in Tables 13A, 13B and 13C (pain), 14A, 14B and 14C (stiffness), 15A, 15B and 15C (physical function) and 16A, 16B and 16C (total score). In each case, the A, B and C tables show the values at Weeks 1, 2 and 3, respectively and the D tables show the baseline values.

TABLE 13A
WOMAC OSTEOARTHRITIS PAIN SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 1 | | | | | |
| N | 50 | 98 | 100 | 100 | 348 |
| MEAN | 311.1 | 263.2 | 292.5 | 271.9 | 281.0 |
| STANDARD DEVIATION | 121.29 | 124.90 | 107.04 | 119.53 | 118.60 |
| MINIMUM | 37.0 | 3.0 | 28.0 | 0.0 | 0.0 |
| MEDIAN | 320.0 | 279.0 | 312.0 | 274.0 | 297.0 |
| MAXIMUM | 500.0 | 483.0 | 492.0 | 488.0 | 500.0 |
| MODEL P-VALUES¹ | | | | | |
| TREATMENT | 0.079 | | | | |
| SEX | 0.380 | | | | |
| TREATMENT + SEX | 0.856 | | | | |
| PAIRWISE | | | | | |
| COMPARISON P-VALUES¹ | | | | | |
| PLACEBO QID | — | 0.029 | 0.531 | 0.088 | |
| OXY QID | — | — | 0.053 | 0.544 | |
| OXY + NTX QID | — | — | — | 0.179 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 13B
WOMAC OSTEOARTHRITIS PAIN SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 101 | 349 |
| MEAN | 285.7 | 245.0 | 257.2 | 237.7 | 252.2 |
| STANDARD DEVIATION | 144.01 | 131.13 | 120.08 | 133.58 | 131.10 |
| MINIMUM | 14.0 | 0.0 | 22.0 | 0.0 | 0.0 |
| MEDIAN | 295.5 | 227.5 | 272.0 | 226.0 | 257.5 |
| MAXIMUM | 500.0 | 482.0 | 492.0 | 494.0 | 500.0 |
| MODEL P-VALUES¹ | | | | | |
| TREATMENT | 0.107 | | | | |
| SEX | 0.559 | | | | |
| TREATMENT + SEX | 0.631 | | | | |
| PAIRWISE | | | | | |
| COMPARISON P-VALUES¹ | | | | | |
| PLACEBO QID | — | 0.064 | 0.230 | 0.020 | |
| OXY QID | — | — | 0.415 | 0.568 | |
| OXY + NTX QID | — | — | — | 0.161 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 13C
WOMAC OSTEOARTHRITIS PAIN SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 101 | 349 |
| MEAN | 269.1 | 238.7 | 245.3 | 225.7 | 241.2 |
| STANDARD DEVIATION | 160.58 | 135.11 | 131.06 | 139.11 | 139.12 |
| MINIMUM | 4.0 | 0.0 | 0.0 | 0.0 | 0.0 |
| MEDIAN | 316.5 | 225.0 | 280.0 | 211.0 | 244.0 |
| MAXIMUM | 500.0 | 488.0 | 492.0 | 495.0 | 500.0 |
| MODEL P-VALUES¹ | | | | | |
| TREATMENT | 0.172 | | | | |
| SEX | 0.155 | | | | |
| TREATMENT + SEX | 0.439 | | | | |
| PAIRWISE | | | | | |
| COMPARISON P-VALUES¹ | | | | | |
| PLACEBO QID | — | 0.154 | 0.354 | 0.035 | |
| OXY QID | — | — | 0.531 | 0.409 | |
| OXY + NTX QID | — | — | — | 0.142 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 13D
WOMAC OSTEOARTHRITIS PAIN SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| BASELINE | | | | | |
| N | 50 | 102 | 102 | 101 | 355 |
| MEAN | 355.1 | 327.9 | 342.9 | 342.7 | 340.3 |
| STANDARD DEVIATION | 80.68 | 90.62 | 71.40 | 80.97 | 81.56 |
| MINIMUM | 134.0 | 25.0 | 148.0 | 86.0 | 25.0 |
| MEDIAN | 363.5 | 338.0 | 340.0 | 350.0 | 347.0 |
| MAXIMUM | 489.0 | 500.0 | 482.0 | 490.0 | 500.0 |
| MODEL P-VALUES¹ | | | | | |
| TREATMENT | 0.371 | | | | |
| SEX | 0.021 | | | | |
| TREATMENT + SEX | 0.925 | | | | |
| PAIRWISE | | | | | |
| COMPARISON P-VALUES¹ | | | | | |
| PLACEBO QID | — | 0.110 | 0.586 | 0.481 | |
| OXY QID | — | — | 0.190 | 0.267 | |
| OXY + NTX QID | — | — | — | 0.842 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14A
WOMAC OSTEOARTHRITIS STIFFNESS SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 1 | | | | | |
| N | 49 | 98 | 100 | 99 | 346 |
| MEAN | 131.0 | 120.3 | 127.4 | 119.0 | 123.5 |
| STANDARD DEVIATION | 50.41 | 51.28 | 44.29 | 51.32 | 49.24 |
| MINIMUM | 8.0 | 2.0 | 6.0 | 0.0 | 0.0 |
| MEDIAN | 139.0 | 129.5 | 131.0 | 128.0 | 130.5 |
| MAXIMUM | 199.0 | 200.0 | 197.0 | 200.0 | 200.0 |
| MODEL P-VALUES¹ | | | | | |
| TREATMENT | 0.274 | | | | |
| SEX | 0.290 | | | | |
| TREATMENT + SEX | 0.656 | | | | |
| PAIRWISE | | | | | |
| COMPARISON P-VALUES¹ | | | | | |
| PLACEBO QID | — | 0.192 | 0.858 | 0.174 | |
| OXY QID | — | — | 0.163 | 0.954 | |
| OXY + NTX QID | — | — | — | 0.142 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14B
WOMAC OSTEOARTHRITIS STIFFNESS SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 100 | 348 |
| MEAN | 128.3 | 111.4 | 115.8 | 106.5 | 113.7 |
| STANDARD DEVIATION | 55.95 | 56.29 | 48.60 | 55.44 | 54.10 |
| MINIMUM | 8.0 | 0.0 | 8.0 | 0.0 | 0.0 |
| MEDIAN | 142.5 | 119.5 | 125.0 | 113.5 | 121.0 |
| MAXIMUM | 200.0 | 199.0 | 197.0 | 200.0 | 200.0 |
| MODEL P-VALUES¹ | | | | | |
| TREATMENT | 0.098 | | | | |
| SEX | 0.249 | | | | |
| TREATMENT + SEX | 0.745 | | | | |
| PAIRWISE | | | | | |
| COMPARISON P-VALUES¹ | | | | | |
| PLACEBO QID | — | 0.061 | 0.238 | 0.019 | |
| OXY QID | — | — | 0.383 | 0.569 | |
| OXY + NTX QID | — | — | — | 0.145 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14C
WOMAC OSTEOARTHRITIS STIFFNESS SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 100 | 348 |
| MEAN | 119.8 | 106.3 | 110.7 | 101.1 | 208.0 |
| STANDARD DEVIATION | 64.12 | 57.99 | 53.42 | 57.66 | 57.62 |
| MINIMUM | 4.0 | 0.0 | 0.0 | 0.0 | 0.0 |
| MEDIAN | 139.5 | 109.0 | 117.5 | 113.5 | 117.5 |
| MAXIMUM | 200.0 | 200.0 | 197.0 | 200.0 | 200.0 |
| MODEL P-VALUES¹ | | | | | |
| TREATMENT | 0.148 | | | | |
| SEX | 0.068 | | | | |
| TREATMENT + SEX | 0.396 | | | | |
| PAIRWISE | | | | | |
| COMPARISON P-VALUES¹ | | | | | |
| PLACEBO QID | — | 0.143 | 0.496 | 0.042 | |
| OXY QID | — | — | 0.328 | 0.491 | |
| OXY + NTX QID | — | — | — | 0.092 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14D
WOMAC OSTEOARTHRITIS STIFFNESS SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| BASELINE | | | | | |
| N | 50 | 102 | 102 | 101 | 355 |
| MEAN | 149.7 | 141.8 | 149.3 | 141.4 | 145.0 |
| STANDARD DEVIATION | 41.75 | 37.28 | 33.50 | 39.35 | 37.56 |
| MINIMUM | 13.0 | 32.0 | 41.0 | 10.0 | 10.0 |
| MEDIAN | 158.5 | 145.5 | 152.0 | 147.0 | 148.0 |
| MAXIMUM | 200.0 | 200.0 | 197.0 | 200.0 | 200.0 |
| MODEL P-VALUES¹ | | | | | |
| TREATMENT | 0.221 | | | | |
| SEX | 0.075 | | | | |
| TREATMENT + SEX | 0.688 | | | | |
| PAIRWISE | | | | | |
| COMPARISON P-VALUES¹ | | | | | |
| PLACEBO QID | — | 0.266 | 0.993 | 0.149 | |
| OXY QID | — | — | 0.170 | 0.686 | |
| OXY + NTX QID | — | — | — | 0.075 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15A
WOMAC OSTEOARTHRITIS PHYSICAL FUNCTION SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 1 | | | | | |
| N | 50 | 98 | 100 | 100 | 348 |
| MEAN | 1034.4 | 891.8 | 998.7 | 932.9 | 954.8 |
| STANDARD DEVIATION | 431.11 | 423.35 | 378.56 | 413.37 | 410.74 |
| MINIMUM | 123.0 | 15.0 | 149.0 | 52.0 | 15.0 |
| MEDIAN | 1098.0 | 930.5 | 1098.5 | 955.5 | 1018.5 |
| MAXIMUM | 1700.0 | 1669.0 | 1658.0 | 1642.0 | 1700.0 |
| MODEL P-VALUES [1] | | | | | |
| TREATMENT | 0.085 | | | | |
| SEX | 0.622 | | | | |
| TREATMENT + SEX | 0.771 | | | | |
| PAIRWISE | | | | | |
| COMPARISON P-VALUES¹ | | | | | |
| PLACEBO QID | — | 0.034 | 0.640 | 0.136 | |
| OXY QID | — | — | 0.040 | 0.424 | |
| OXY + NTX QID | — | — | — | 0.204 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15B
WOMAC OSTEOARTHRITIS PHYSICAL FUNCTION SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 101 | 349 |
| MEAN | 985.0 | 847.6 | 910.6 | 834.4 | 881.5 |
| STANDARD DEVIATION | 461.89 | 451.42 | 410.69 | 439.16 | 439.31 |
| MINIMUM | 57.4 | 16.0 | 54.0 | 17.0 | 16.0 |
| MEDIAN | 1038.0 | 880.0 | 979.5 | 799.0 | 888.0 |
| MAXIMUM | 1700.0 | 1669.0 | 1658.0 | 1646.0 | 1700.0 |
| MODEL P-VALUES¹ | | | | | |
| TREATMENT | 0.099 | | | | |
| SEX | 0.457 | | | | |
| TREATMENT + SEX | 0.455 | | | | |
| PAIRWISE | | | | | |
| COMPARISON P-VALUES¹ | | | | | |
| PLACEBO QID | — | 0.070 | 0.471 | 0.036 | |
| OXY QID | — | — | 0.176 | 0.738 | |
| OXY + NTX QID | — | — | — | 0.088 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15C
WOMAC OSTEOARTHRITIS PHYSICAL FUNCTION SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 101 | 349 |
| MEAN | 939.9 | 820.0 | 869.0 | 801.9 | 846.0 |
| STANDARD DEVIATION | 534.35 | 468.67 | 452.25 | 458.64 | 471.41 |
| MINIMUM | 9.6 | 5.0 | 0.0 | 17.0 | 0.0 |
| MEDIAN | 1004.0 | 867.0 | 969.5 | 776.0 | 890.0 |
| MAXIMUM | 1700.0 | 1680.0 | 1662.0 | 1671.0 | 1700.0 |
| MODEL P-VALUES¹ | | | | | |
| TREATMENT | 0.176 | | | | |
| SEX | 0.238 | | | | |
| TREATMENT + SEX | 0.380 | | | | |
| PAIRWISE | | | | | |
| COMPARISON P-VALUES¹ | | | | | |
| PLACEBO QID | — | 0.131 | 0.489 | 0.052 | |
| OXY QID | — | — | 0.307 | 0.604 | |
| OXY + NTX QID | — | — | — | 0.120 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15D
WOMAC OSTEOARTHRITIS PHYSICAL FUNCTION SUBSCALE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| BASELINE | | | | | |
| N | 49 | 101 | 102 | 101 | 353 |
| MEAN | 1146.7 | 1101.7 | 1147.8 | 1142.9 | 1133.1 |
| STANDARD DEVIATION | 341.34 | 324.42 | 302.25 | 304.85 | 314.33 |
| MINIMUM | 208.0 | 208.0 | 199.0 | 273.0 | 199.0 |
| MEDIAN | 1180.0 | 1151.0 | 1187.5 | 1210.0 | 1187.0 |
| MAXIMUM | 1688.0 | 1700.0 | 1659.0 | 1650.0 | 1700.0 |
| MODEL P-VALUES¹ | | | | | |
| TREATMENT | 0.837 | | | | |
| SEX | 0.020 | | | | |
| TREATMENT + SEX | 0.936 | | | | |
| PAIRWISE | | | | | |
| COMPARISON P-VALUES¹ | | | | | |
| PLACEBO QID | — | 0.474 | 0.929 | 0.864 | |
| OXY QID | — | — | 0.435 | 0.498 | |
| OXY + NTX QID | — | — | — | 0.918 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16A
WOMAC OSTEOARTHRITIS TOTAL SCORE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 1 | | | | | |
| N | 49 | 98 | 100 | 99 | 346 |
| MEAN | 1468.6 | 1275.3 | 1418.5 | 1316.8 | 1355.9 |
| STANDARD DEVIATION | 592.38 | 582.28 | 511.30 | 565.78 | 561.54 |
| MINIMUM | 214.0 | 20.0 | 227.0 | 55.0 | 20.0 |
| MEDIAN | 1539.0 | 1312.5 | 1552.5 | 1323.0 | 1428.0 |
| MAXIMUM | 2398.0 | 2347.0 | 2340.0 | 2328.0 | 2398.0 |
| MODEL P-VALUES¹ | | | | | |
| TREATMENT | 0.085 | | | | |
| SEX | 0.576 | | | | |
| TREATMENT + SEX | 0.782 | | | | |
| PAIRWISE | | | | | |
| COMPARISON P-VALUES¹ | | | | | |
| PLACEBO QID | — | 0.039 | 0.664 | 0.119 | |
| OXY QID | — | — | 0.042 | 0.516 | |
| OXY + NTX QID | — | — | — | 0.162 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16B
WOMAC OSTEOARTHRITIS TOTAL SCORE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 100 | 348 |
| MEAN | 1399.0 | 1204.0 | 1283.0 | 1170.3 | 1245.0 |
| STANDARD DEVIATION | 649.08 | 626.06 | 557.71 | 611.83 | 608.70 |
| MINIMUM | 81.4 | 20.0 | 99.0 | 55.0 | 20.0 |
| MEDIAN | 1483.0 | 1222.5 | 1309.0 | 1137.5 | 1257.0 |
| MAXIMUM | 2400.0 | 2347.0 | 2346.0 | 2338.0 | 2400.0 |
| MODEL P-VALUES¹ | | | | | |
| TREATMENT | 0.082 | | | | |
| SEX | 0.463 | | | | |
| TREATMENT + SEX | 0.546 | | | | |
| PAIRWISE | | | | | |
| COMPARISON P-VALUES¹ | | | | | |
| PLACEBO QID | — | 0.061 | 0.372 | 0.023 | |
| OXY QID | — | — | 0.222 | 0.635 | |
| OXY + NTX QID | — | — | — | 0.087 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16C
WOMAC OSTEOARTHRITIS TOTAL SCORE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 100 | 348 |
| MEAN | 1328.8 | 1164.9 | 1225.0 | 1121.0 | 1193.1 |
| STANDARD DEVIATION | 750.67 | 651.16 | 624.27 | 638.73 | 656.01 |
| MINIMUM | 17.6 | 6.0 | 0.0 | 55.0 | 0.0 |
| MEDIAN | 1468.0 | 1191.0 | 1312.0 | 1143.0 | 1231.5 |
| MAXIMUM | 2400.0 | 2368.0 | 2348.0 | 2364.0 | 2400.0 |
| MODEL P-VALUES¹ | | | | | |
| TREATMENT | 0.149 | | | | |
| SEX | 0.201 | | | | |
| TREATMENT + SEX | 0.410 | | | | |
| PAIRWISE | | | | | |
| COMPARISON P-VALUES¹ | | | | | |
| PLACEBO QID | — | 0.129 | 0.451 | 0.039 | |
| OXY QID | — | — | 0.341 | 0.511 | |
| OXY + NTX QID | — | — | — | 0.204 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16D
WOMAC OSTEOARTHRITIS TOTAL SCORE - BY WEEK
ANALYSIS POPULATION: INTENT TO TREAT POPULATION

| | PLACEBO | OXY | OXY + NTX | OXY + NTX | |
|---|---|---|---|---|---|
| | QID | QID | QID | BID | TOTAL |
| | (N = 50) | (N = 102) | (N = 102) | (N = 102) | (N = 356) |
| BASELINE | | | | | |
| N | 49 | 101 | 102 | 101 | 353 |
| MEAN | 1650.2 | 1569.6 | 1640.1 | 1627.0 | 1617.6 |
| STANDARD DEVIATION | 445.37 | 432.36 | 382.02 | 397.20 | 409.67 |
| MINIMUM | 421.0 | 328.0 | 570.4 | 397.0 | 328.0 |
| MEDIAN | 1677.0 | 1640.0 | 1659.0 | 1703.0 | 1662.0 |
| MAXIMUM | 2377.0 | 2400.0 | 2330.0 | 2273.0 | 2400.0 |
| MODEL P-VALUES¹ | | | | | |
| TREATMENT | 0.704 | | | | |
| SEX | 0.017 | | | | |
| TREATMENT + SEX | 0.949 | | | | |
| PAIRWISE | | | | | |
| COMPARISON P-VALUES¹ | | | | | |
| PLACEBO QID | — | 0.330 | 0.867 | 0.694 | |
| OXY QID | — | — | 0.315 | 0.469 | |
| OXY + NTX QID | — | — | — | 0.778 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

The overall incidences of adverse events in all three active treatment groups were generally comparable, and the numerical differences observed are shown in the following tables. The most frequent adverse events (AEs) reported were those commonly associated with opioid medications: dizziness, constipation, dry mouth, nausea, vomiting, somnolence, and pruritis. Table 17 shows adverse events experienced by ≧5% of the patients during Weeks 1, 2, or 3 of treatment, based on the Intent To Treat Population.

TABLE 17
Adverse Events
Number (%) of Patients

| | | | | | Oxycodone Plus | | Oxycodone Plus | |
|---|---|---|---|---|---|---|---|---|
| | | | Oxycodone QID | | Naltrexone QID | | Naltrexone BID | |
| Adverse Event | Placebo | | Days 1-3: 10 mg OXY/d | | Days 1-3: 10 mg OXY/d | | Days 1-3: 10 mg OXY/d | |
| Week 1, | Placebo | | Days 4-8: 20 mg OXY/d | | Days 4-8: 20 mg OXY/d | | Days 4-8: 20 mg OXY/d | |
| Dose N | QID 51 | | 102 | | 104 | | 103 | |
| Constipation | 2 | (3.9) | 13 | (12.7) | 5 | (4.8) | 11 | (10.7) |
| Dry Mouth | 0 | (0.0) | 6 | (5.9) | 6 | (5.8) | 7 | (6.8) |
| Nausea | 4 | (7.8) | 24 | (23.5) | 12 | (11.5) | 28 | (27.2) |
| Vomiting | 2 | (3.9) | 4 | (3.9) | 2 | (1.9) | 8 | (7.8) |
| Fatigue | 2 | (3.9) | 3 | (2.9) | 7 | (6.7) | 3 | (2.9) |
| Dizziness | 0 | (0.0) | 17 | (16.7) | 16 | (15.4) | 31 | (30.1) |
| Headache | 7 | (13.7) | 17 | (16.7) | 13 | (12.5) | 12 | (11.7) |
| Sommolence | 2 | (3.9) | 16 | (15.7) | 12 | (11.5) | 12 | (11.7) |
| Pruritus | 2 | (3.9) | 8 | (7.8) | 3 | (2.9) | 10 | (9.7) |

Number (%) of Patients

| | | | | |
|---|---|---|---|---|
| | | | Oxycodone Plus | Oxycodone Plus |
| Adverse Event | Placebo | Oxycodone QID | Naltrexone QID | Naltrexone BID |
| Week 2, | Placebo | 30 mg OXY/d | 30 mg OXY/d | 30 mg OXY/d |
| Dose N | QID 45 | 85 | 84 | 74 |

| | | | | | | | | |
|---|---|---|---|---|---|---|---|---|
| Constipation | 0 | (0.0) | 6 | (7.1) | 15 | (17.9) | 9 | (12.2) |
| Dry Mouth | 0 | (0.0) | 4 | (4.7) | 2 | (2.4) | 5 | (6.8) |
| Nausea | 1 | (2.2) | 12 | (14.1) | 3 | (3.6) | 10 | (13.5) |
| Vomiting | 0 | (0.0) | 3 | (3.5) | 1 | (1.2) | 4 | (5.4) |
| Fatigue | 3 | (6.7) | 0 | (0.0) | 1 | (1.2) | 1 | (1.4) |
| Dizziness | 0 | (0.0) | 10 | (11.8) | 10 | (11.9) | 7 | (9.5) |
| Headache | 3 | (6.7) | 3 | (3.5) | 5 | (6.0) | 3 | (4.1) |
| Somnolence | 1 | (2.2) | 6 | (7.1) | 6 | (7.1) | 5 | (6.8) |
| Pruritus | 0 | (0.0) | 5 | (5.9) | 3 | (3.6) | 5 | (6.8) |

| | | | | |
|---|---|---|---|---|
| Week 3, | Placebo | 40 mg OXY/d | 40 mg OXY/d | 40 mg OXY/d |
| Dose N | QID 42 | 75 | 74 | 61 |

| | | | | | | | | |
|---|---|---|---|---|---|---|---|---|
| Constipation | 1 | (2.4) | 2 | (2.7) | 9 | (12.2) | 3 | (4.9) |
| Dry Mouth | 0 | (0.0) | 0 | (0.0) | 3 | (4.1) | 4 | (6.6) |
| Nausea | 2 | (4.8) | 8 | (10.7) | 8 | (10.8) | 6 | (9.8) |
| Vomiting | 1 | (2.4) | 9 | (12.0) | 2 | (2.7) | 2 | (3.3) |
| Dizziness | 0 | (0.0) | 4 | (5.3) | 6 | (8.1) | 2 | (3.3) |
| Headache | 3 | (7.1) | 6 | (8.0) | 1 | (1.4) | 3 | (4.9) |
| Sommolence | 1 | (2.4) | 2 | (2.7) | 8 | (10.8) | 5 | (8.2) |
| Pruritus | 0 | (0.0) | 3 | (4.0) | 1 | (1.4) | 7 | (11.5) |

Seventy-nine of the 360 patients who received study drug-discontinued treatment because of adverse events (0 placebo; 29 oxycodone alone QID; 18 oxycodone plus naltrexone QID and 32 oxycodone plus naltrexone BID). Oxycodone plus naltrexone QID had the lowest AE discontinuation rate among the 3-active treatment group while oxycodone plus naltrexone BID and oxycodone alone QID were comparable. Adverse events that resulted in the discontinuation of treatment in two patients or more in any treatment group are shown in Table 18 below, based on the Intent to Treat Population. Most of the adverse events that resulted in treatment discontinuation are commonly associated with the use of opioid analgesics, e.g., nausea, vomiting, constipation, dizziness and somnolence.

TABLE 18
Selected Adverse Events
Number (%) of Patients

| | | | Oxycodone Plus | Oxycodone Plus |
|---|---|---|---|---|
| System Organ Class | Placebo | Oxycodone QID | Naltrexone QID | Naltrexone BID |
| Adverse Event | N = 51 | N = 102 | N = 104 | N = 103 |
| Any adverse event | 0 (0.0) | 29 (28.4) | 18 (17.3) | 32 (31.1) |
| Eye disorders | 0 (0.0) | 0 (0.0) | 2 (1.8) | 0 (0.0) |
| Gastrointestinal disorders | 0 (0.0) | 13 (12.7) | 7 (6.7) | 17 (16.5) |
| Upper abdominal pain | 0 (0.0) | 0 (0.0) | 0 (0.0) | 2 (1.9) |
| Constipation | 0 (0.0) | 1 (1.0) | 0 (0.0) | 2 (1.9) |
| Diarrhea | 0 (0.0) | 0 (0.0) | 2 (1.9) | 0 (0.0) |
| Nausea | 0 (0.0) | 7 (6.9) | 4 (3.8) | 12 (11.7) |
| Vomiting | 0 (0.0) | 4 (3.9) | 1 (1.0) | 3 (2.9) |
| General disorders and | 0 (0.0) | 2 (2.0) | 0 (0.0) | 2 (1.9) |
| administration site conditions | | | | |
| Lethargy | 0 (0.0) | 0 (0.0) | 0 (0.0) | 2 (1.9) |
| Nervous system disorders | 0 (0.0) | 8 (7.8) | 11 (10.6) | 14 (13.6) |
| Dizziness | 0 (0.0) | 1 (1.0) | 5 (4.8) | 9 (8.7) |
| Headache | 0 (0.0) | 2 (2.0) | 2 (1.9) | 0 (0.0) |
| Sedation | 0 (0.0) | 2 (2.0) | 0 (0.0) | 0 (0.0) |
| Sommolence | 0 (0.0) | 2 (2.0) | 5 (4.8) | 5 (4.9) |
| Psychiatric disorders | 0 (0.0) | 1 (1.0) | 3 (2.9) | 0 (0.0) |
| Euphoric mood | 0 (0.0) | 0 (0.0) | 2 (1.9) | 0 (0.0) |
| Skin and subcutaneous | 0 (0.0) | 3 (2.9) | 1 (1.0) | 2 (1.9) |
| tissue disorders | | | | |
| Pruritus | 0 (0.0) | 1 (1.0) | 1 (1.0) | 2 (1.9) |

Serious adverse events (SAES) were reported for five patients. All of the serious adverse events were unrelated to treatment with study medication.

The study was also designed to investigate potential opioid withdrawal effects when patients stopped study drug without tapering at the end of treatment. The Short Opioid Withdrawal Scale (SOWS) (see Table 6 above), originally used for collecting withdrawal data from heroin addicts, was used to assess opioid withdrawal in this study. Although there were statistically significant differences between treatment groups, the differences were considered clinically insignificant because both the mean SOWS changes and the differences of their changes were of small magnitude. The lack of clinically significant opioid withdrawal in this study is attributable to the relatively low opioid doses and short treatment duration. Opioid withdrawal was not reported as an adverse event in any of the treatment groups.

In summary, in this study oxycodone plus naltrexone BID was shown to be a safe and effective treatment for patients with chronic pain and with osteoarthritis of the hip or knee. Oxycodone plus naltrexone BID provided statistically and clinically significant reductions in pain intensity compared to oxycodone alone QID when the same total daily dose of oxycodone was administered. The overall incidence of opioid-related adverse events was comparable in the oxycodone plus naltrexone and oxycodone alone treatment groups and no clinically meaningful effects on vital signs, laboratory safety tests or QTc interval changes were noted in the oxycodone plus naltrexone or oxycodone alone treatment groups. Oxycodone plus naltrexone BID provided better daily pain control to that of oxycodone alone QID. Oxycodone plus naltrexone BID showed greater improvements in all categories of the WOMAC Osteoarthritis Index (pain, stiffness and physical function) when compared to the other active treatment groups.

B.

An additional clinical study was designed substantially the same as that described in Part A, with differences indicated below.

The clinical study was designed as follows: (1) to evaluate the efficacy and safety of combinations of oxycodone (oxy) and naltrexone (ntx) when compared to oxycodone, (2) to evaluate the efficacy and safety of combinations of oxycodone (oxy) and naltrexone (ntx) administered when compared to naltrexone, and (3) to compare the quality of life measures (WOMAC and SF-12) between treatment groups.

A multicenter, randomized, double-blind, active- and placebo-controlled, clinical study was designed and is conducted. The study evaluates the efficacy and safety of an oral formulation of oxycodone and naltrexone relative to oxycodone and to naltrexone over a 12-week fixed-dose period following one week of titration (instead of a three week period). A total of 750 patients (instead of 360 patents) with chronic pain due to osteoarthritis of the hip or knee are enrolled into six (instead of four) treatment groups: three groups for combinations of oxycodone and naltrexone, a group for oxycodone alone, a group for naltrexone alone and a group for placebo.

Patients are randomly assigned to one of the six treatment groups as shown in Table 19.

TABLE 19

| Treatment | | Weeks 1-12 |
|---|---|---|
| Group | Titration | (Fixed-Dose) |
| A | (OXY 10 mg + NTX | (OXY 20 mg + NTX |
| | 0.001 mg) BID | 0.001 mg) BID |
| B | (OXY 5 mg + NTX | (OXY 10 mg + NTX |
| | 0.001 mg) QID | 0.001 mg) QID |
| C | (OXY 10 mg + NTX | (OXY 10 mg + NTX |
| | 0.001 mg) BID | 0.001 mg) BID |
| D | OXY 5 mg QID | OXY 10 mg QID |
| E | NTX 0.001 mg BID | NTX 0.001 mg BID |
| F | Placebo QID | Placebo QID |

All treatment groups are scheduled for QID dosing to protect the double-blind study design as shown in Table 20.

TABLE 20

| Treatment | QID Daily Dosing Scheme* | | | |
|---|---|---|---|---|
| Group | Upon Waking | Noon | Afternoon | Bedtime |
| A | (OXY + NTX) | Placebo | (OXY + NTX) | Placebo |
| B | (OXY + NTX) | (OXY + | (OXY + NTX) | (OXY + |
| | | NTX) | | NTX) |
| C | (OXY + NTX) | Placebo | (OXY + NTX) | Placebo |
| D | OXY | OXY | OXY | OXY |
| E | NTX | Placebo | NTX | Placebo |
| F | Placebo | Placebo | Placebo | Placebo |

*Doses must be taken 30-60 minutes before meals and at least 4 hours apart.

Patients return to the clinic for weekly visits (±one day) for the first five weeks and then at 2-week (±two days) intervals for the remainder of the study (instead of the visit schedule described in Part A). At each clinic visit, quality of analgesia, pain control, and a global assessment of study medication are collected as described above. The SF-12 Health Survey and the WOMAC Osteoarthritis Index are collected monthly (instead of at each clinic visit).

Safety is evaluated as described in Part A.

The Study Population is seven hundred fifty (750) patients with moderate to severe chronic pain due to osteoarthritis of the hip or knee. According to the study design described above, there are 150 patients each in the oxycodone and naltrexone BID, oxycodone and naltrexone QID and oxycodone alone treatment groups; and 75 patients each in the naltrexone and placebo groups.

The inclusion criteria are essentially the same as described above in Part A, with the following exceptions:
Patient agrees to refrain from taking any pain medications other than study drug during the 13-week treatment period, rather than the shorter treatment period of the clinical study of Part A. (Aspirin [up to 325 mg/day] is permitted for cardiovascular prophylaxis if at a stable dose one month prior to the Screening Visit.); and
Patient is able to ambulate for a specified distance (at least 100 meters).

The exclusion criteria are essentially the same as described above in Part A, with the exceptions listed below. Additional exclusion criteria are as follows:
(a) Patient has a positive urine drug screen at the Baseline/Titration Visit NOT caused by any therapeutic medication permitted during the study;
(b) Patient has pain in the hip(s) or knee(s) caused by conditions other than osteoarthritis, e.g., malignancy, gout, inflammatory disease such as rheumatoid arthritis, trauma, fibromyalgia, bony fracture, or infection;
(c) Patient has a history of cardiac disease (such as coronary artery disease, cardiomyopathy, congestive heart disease, valvular disease, arrythmia, etc.), angina, myocardiac infarct (MI), cerebral aneurysm, cerebral vascular accident (CVA), transient ischemic event (TIA), inadequately controlled hypertension, or health condition(s) which poses significant health risk in the event of opioid withdrawal;
(d) Patient has started or stopped physical therapy, transcutaneous electrical nerve stimulation, chiropractic, osteopathic, acupuncture, or other complementary treatment within the past four weeks or is expected to undergo any changes in these therapies in the duration of the study;
(e) Patient has a psychiatric illness or medical illness/condition, and/or abnormal diagnostic finding, that, in the opinion of the investigator, would interfere with the completion of the study, confound the results of the study, or pose risk to the patient;
(f) Patient has a history of the following neoplastic disease: leukemia, lymphoma, or myeloproliferative disease, metastatic cancer. In patients with treated localized malignancies, the decision to exclude is made by the Sponsor on individual cases; and
(g) Patient has AST, ALT, GGT, or alkaline phosphatase>2 times the upper limit of normal; hematocrit<30%; or creatinine≧1.8 at the Screening Visit.

The following exclusion criteria in the clinical study describe of Part A were modified or omitted for the present clinical study:
(a) Acute hepatitis is not included in the exclusion factors;
(b) History of severe hepatic or renal impairment is not included in the exclusion factors;
(c) “Patient had other diseases significant enough, in the opinion of the Investigator, to pose a risk for the administration of study drug or that will interfere with pain assessments,” is not included in the exclusion factors;
(d) Patient has a history of gastric, biliary, or small intestine surgery, or any other diseases that cause clinical malabsorption is an exclusion factor (rather than “Patient had chronic biliary tract disease, chronic pancreatitis, or inflammatory bowel disorders”); and
(e) Patient's history of alcohol or drug abuse is within the past 5 years;

The physical descriptions of the drugs used for the study are as follows. For the washout period, acetaminophen is dispensed as described in Part A. The investigational drug supplies are in tablet dosage forms containing oxycodone HCl and naltrexone HCl, oxycodone HCl, naltrexone or placebo. All of the tablet dosage forms are indistinguishable from one another to facilitate blinding. Tablets are arranged on each blister card by Study Day and contain four doses per day. Each blister card also contains three days of extra study drug to allow for flexibility in planning return clinic visits. The extra study drug must remain intact within its original packaging so that it may be returned during each clinic visit. The investigational drug supplies are dispensed in these kits.

Safety procedures include those described in Part A. The opioid toxicity assessment includes: (A) CNS review by assessing for (1) confusion, altered mental state, (2) excessive drowsiness, lethargy, stupor, (3) slurred speech (new onset), (B) respiratory review by assessing for (1) hypoventilation, shortness of breath, apnea, (2) decreased respiratory rate (<8) or cyanosis; and (3) cardiac review by assessing for heart rate<60, hypotension. If patients must be terminated from the study, the Early Drug Termination assessments and opioid withdrawal monitoring (as needed) are performed as discussed below.

At the first visit, pre-enrollment screening is performed as described in Part A.

The second visit is on the first day of the first titration period of the study. The patients returned to the study center four to seven days after the Screening Visit for completion of the pre-dose assessments. This visit included (1) obtaining a urine sample for drug screening using a rapid drug screen kit (BioChek iCup™ Drug Screen). If positive for any drugs not caused by any therapeutic medication permitted during the study, no further assessments are made. Patient cannot continue in the study; (2) reviewing the take-home diary from the past four to seven days; (3) collect bottle of acetaminophen and perform accountability, (4) baseline clinic PI rating; and (5) reviewing inclusion and exclusion criteria. This assessment also includes verifying that (a) the mean daily overall pain intensity score collected in the diary over the last two days of the 4- to 7-day washout period is ≧5 (on a scale of 0 to 10) while off all analgesic medications (except acetaminophen as directed); (b) the clinic PI at this visit measures≧5 (on a scale of 0 to 10); and (c) checking that the clinical laboratory tests results from the screening visit are without significant clinical abnormalities, and that the urine pregnancy test is negative (if required).

Patients meeting the study entry criteria are randomly assigned to one of the six treatment groups, and are assigned a randomization number and study medication kit number. The following assessments are then conducted: (1) a brief (interim) medical history; (2) vital signs; (3) review and record concomitant medications; (4) SF-12 Health Survey; and (5) WOMAC Osteoarthritis Index.

Once these assessments and procedures are completed, the study medication kit is dispensed for the titration period. The patients received their take-home daily diaries and are provided with an appointment card for the next visit. The study nurse thoroughly reviewed each section of the diary with the patient. The daily diary issued at Visit 2 is used to record the following information at bedtime immediately prior to dosing: (1) overall PI in the past 24 hours; and (2) adverse events.

Patients return to the study center at the end of titration (±one day) for the following:
(1) opioid toxicity assessment;
(2) review take-home diary (including overall daily bedtime PI and opioid-related adverse events);
(3) record new/changed adverse events and concomitant medications;
(4) collect study medication from previous week and account for used/unused supplies;
(5) vital signs;
(6) quality of analgesia;
(7) pain control;
(8) global assessment of study medication;
(9) dispense take-home daily diary; and
(10) dispense one blister card of study medication (by telephoning IVRS).

At the conclusion of this visit, the patient is given an appointment card for the next study visit.

Patients return to the study center at weekly intervals (±one day) for 4 weeks (Visits 4-7) and at the end of Weeks 6, 8, and 10 (±two days) (Visits 8-10) for the assessments:
(1) opioid toxicity assessment;
(2) review take-home diary (including overall daily bedtime PI and opioid-related adverse events);
(3) record new/changed adverse events and concomitant medications;
(4) collect study medication from previous week and account for used/unused supplies;
(5) vital signs;
(6) quality of analgesia;
(7) pain control;
(8) global assessment of study medication;
(9) SF-12 Health Survey;
(10) WOMAC Osteoarthritis Index;
(11) dispense take-home daily diary;
(12) dispense one blister card of study medication (by telephoning IVRS). Two blister cards are dispensed at the End of Week 4 Visit.

At the conclusion of each visit, the patient is given an appointment card for the next study visit.

Patients return to the study center at either the end of Week 12 (±two days) or after early drug termination for the same End of Treatment assessments described above except that a blood sample for PK analysis is not taken, and SOWS is only performed if the subject is on the study drug≧4 weeks.

At the conclusion of this visit, prior to departing the center, the patient is given an appointment card for the next study visit. Patients are instructed to contact the study center immediately if they experience severe signs and symptoms of opioid withdrawal.

For four days after the last dose of study medication, the study center contacts patients as described in Part A to monitor for symptoms of opioid withdrawal.

Patients return to the study center approximately one week (±two days) after the last dose of study medication for a post-treatment follow-up visit (Visit 12). At this visit, the following assessments are completed:
(1) review take-home diary; and
(2) record new/changed adverse events and concomitant medications.

Patients may choose to discontinue study drug or study participation at any time, for any reason, specified or unspecified, and without prejudice. If a patient chooses to discontinue study drug early, the investigator must request that the patient return to the clinic within 24 hours of stopping the study medication and complete the end of study assessments described above. For patients who have been on study medication for ≧4 weeks, Day 1 of the opioid withdrawal monitoring period begins 24 hours after the last dose of study medication. The investigator can request that the patient remain in the study for the post-treatment follow-up visit. Study drug assigned to patients who discontinue early may not be reassigned.

The primary analysis population is the intent-to-treat (ITT) population. The ITT population will consist of all patients who take study medication and are used for both efficacy and safety analyses. In the event that a patient is randomized incorrectly or is administered the incorrect study medication, the patient is analyzed according to the study drug actually received. Additional analysis populations may be defined as appropriate based on the actual study experience.

Demographic variables and patient characteristics are summarized descriptively by treatment group and overall. Demographic variables will include age, weight, height, gender, and race/ethnicity. Baseline characteristics includes the PI score recorded in the clinic and baseline values of efficacy variables. Baseline and post-baseline patient characteristics includes study drug administration and prior and concomitant medications.

The following endpoints are summarized and analyzed:
(1) Daily diary PI score—The daily PI assessments are analyzed as weekly values as follows. For each post-baseline week, the PI recorded during the last two days of dosing within the week are averaged. Baseline PI is defined as the average PI recorded during the two days immediately prior to the Baseline/Titration visit;
(2) Quality of analgesia—assessed and analyzed by clinic visit (weekly for the first five weeks and biweekly thereafter);
(3) Pain control—assessed and analyzed by clinic visit (weekly for the first five weeks and biweekly thereafter);
(4) Global assessment of study medication—assessed and analyzed by clinic visit (weekly for the first five weeks and biweekly thereafter);
(5) SF-12 Health Survey—assessed and analyzed monthly; scored as described in the documentation (e.g., Ware et al., 1998); and
(6) WOMAC Osteoarthritis Index—assessed and analyzed monthly; calculated per the WOMAC User Guide.

Missing efficacy data is imputed using the last-observation-carried-forward (LOCF) approach. If the number of patients per center is small, centers may be pooled for analysis, or omitted from statistical models. Unless otherwise indicated, all testing of statistical hypotheses is two-sided, and a difference resulting in a p-value of less than or equal to 0.05 is considered statistically significant.

For primary analysis of data, the primary efficacy endpoint is the percent change from baseline to Visit 11 (Week 12 or early drug termination) in average daily PI. An analysis of covariance (ANCOVA) model is employed, as described below. The pairwise treatment comparison that is of primary interest is treatment group A ([OXY 20 mg+NTX 0.001 mg] during the fixed-dose period) vs. treatment group D (OXY 10 mg QID during the fixed-dose period).

For secondary analysis of data, the average daily PI, SF-12 Health Survey, and WOMAC Osteoarthritis Index is analyzed in terms of the values themselves as well as in terms of change and percent change from baseline. These variables are summarized descriptively by treatment group and by sex. Treatments are compared globally and in pairwise fashion at each time point using an analysis of covariance (ANCOVA) model that includes treatment, center, and sex as factors and baseline value as a covariate. Potential interactions are assessed by also fitting a model with the same main effects and with the treatment by center, treatment by sex, and treatment by baseline interaction terms. In addition, pairwise t-tests are used to compare each post-baseline time point to each prior time point, within treatment group, overall and by sex.

The quality of analgesia, pain control, and global assessment of study medication are summarized descriptively by treatment group, overall and by sex. Treatments are compared at each time point using the Cochran-Mantel-Haenszel row mean scores test, using equally spaced scores, stratified by sex. Cochran-Mantel-Haenszel row mean scores tests will also be used to compare each post-baseline time point to each prior time point, within treatment group, overall and by sex.

Sensitivity analyses are carried out to determine the extent to which the statistical analysis results are influenced by the choice to impute missing observations using LOCF. The primary analysis is repeated using one or more alternative imputation methods (e.g., imputing data following withdrawal depending on the reason for withdrawal) and using an appropriate longitudinal analysis technique such as repeated measures mixed-effects analysis of variance. In addition, an “observed data” analysis, without any data imputation, is conducted on selected endpoints using the same analysis methods described previously.

Adverse events are reported and examined as described in Part A. Change from baseline is summarized descriptively for vital signs and QTc interval. Laboratory data is summarized descriptively on the original scale, change from baseline, and in terms of the normal range.

### EXAMPLE 2

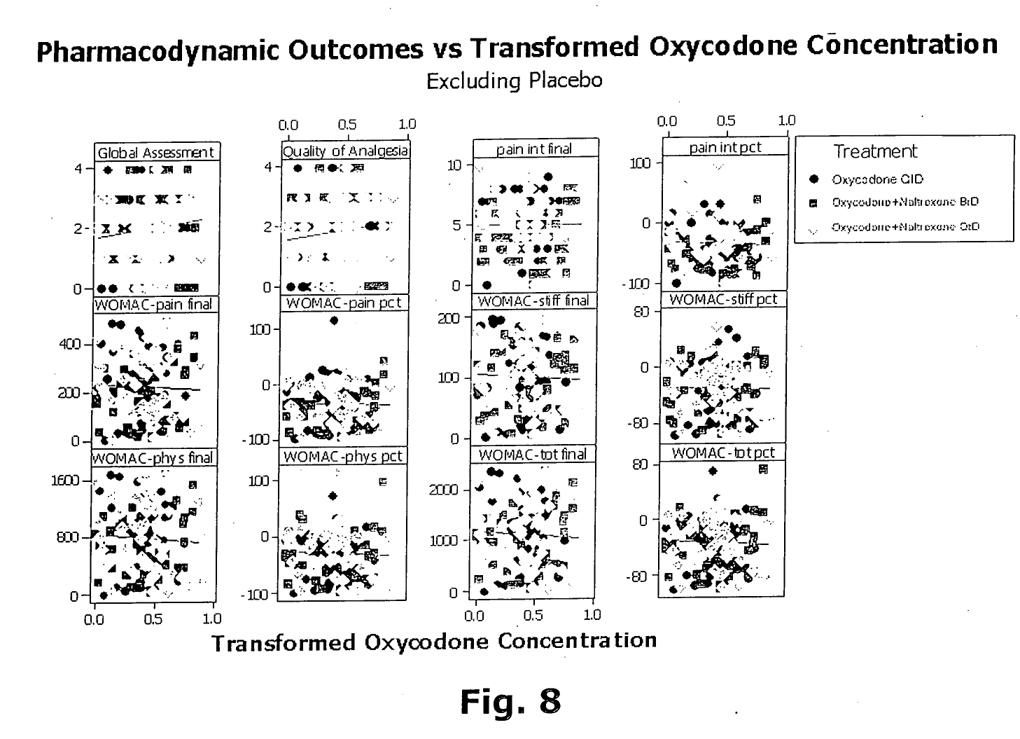

A clinical study was conducted as described in Example 1 wherein safety and analgesic effects of oxycodone or a combination of oxycodone and naltrexone were measured in patients with chronic pain as described in Example 1. Plasma concentrations of the administered drugs and their major metabolites were measured to determine: (1) oxycodone absorption from the combination drug of oxycodone and naltrexone; (2) dose proportionality of plasma concentrations of oxycodone and oxymorphone from the combination drug of oxycodone and naltrexone; (3) achievement of steady state of plasma concentrations of oxycodone, oxymorphone and 6β-naltrexol from the combination drug of oxycodone and naltrexone; and (4) consistency of the half-life and clearance of oxycodone over the course of the study. The relationships between clinical outcomes and the plasma concentrations of oxycodone, oxymorphone, and 6β-naltrexol were plotted for each treatment as shown in FIGS. 8 to 10.

Patients with moderate-to-severe pain due to osteoarthritis of the hip or knee were randomly assigned to one of four treatment groups as shown in Table 3 of Example 1. Plasma samples were obtained for each patient at the beginning of Weeks 1, 2, 3 and at the end of dosing during Week 3.

Patients for inclusion in the bioanalytical analyses (24/sex/treatment arm) were randomly selected from those who completed all three weeks of treatment in each of the three active treatment arms. Plasma samples randomly selected from each of those treatment arms were analyzed for oxycodone, oxymorphone, noroxycodone, naltrexone and 6β-naltrexol by validated coupled solid phase extraction LC-MS/MS methods.

For the analysis of linearity and dose proportionality, linear equation coefficients were obtained by averaging patient-specific slopes and intercepts obtained by within-patient least squares regression. This was done to account for the correlation among the repeated measurements due to the patient's contributing data at each dose level. The resulting slopes among treatment groups were compared by one-way analysis of variance (ANOVA), and a one-sample t-test assessed the common slope's difference from zero. A measure of deviation from linearity was constructed as the difference between the concentration at the middle dose versus the average of the concentrations at the lower and higher doses. Due to equal spacing of doses, this measure has expectation zero under the hypothesis of linearity. As above, ANOVA and t-tests were used to assess linearity.

The relationship between oxycodone plasma concentration and various outcome measures were assessed by Pearson correlation coefficients and associated p-values. For these analyses, the plasma concentration data were log-transformed in order to achieve approximately Gaussian distributions. Oxycodone plasma concentrations (ignoring time of blood draw) in active treatment arms were compared by one-way ANOVA. Regression analyses combined with F-tests on the extra sums of squares assessed whether profiles and correlations differed among active treatment arms. P-values were computed without adjustment for multiple testing. Similar analyses were conducted on the oxymorphone and 6β-naltrexol plasma concentrations.

The oxycodone and oxymorphone plasma concentration data showed a skewed distribution commonly seen in pharmacokinetic data. The base-10 log transformation reduced the skewness on the right tail (larger concentrations) but introduced skewness on the lower tail. To achieve symmetry, modified log transformations were used. Symmetry was achieved using the following modified log transformations:
Transformed oxycodone value=log(original value+10)−log(10)
Transformed oxymorphone value=log(original value+0.1)−log(0.1)
Transformed 6β-naltrexol value=log(original value).

The translations by −log(10) and −log(0.1) were used so that concentrations of zero on the original scale would be transformed to zero. The transformation for 6β-naltrexol did not require a translation to achieve an approximately Gaussian distribution.

In addition to summary statistics and graphs, box-and-whisker plots were used to summarize the distribution of variables. Those figures depict either the minimum value in the data or selected outliers at the lower end, the quartile (25th percentile), the median, the upper quartile (75th percentile) and the maximum or selected outliers at the upper end.

Statistical analyses except extra sum of squares analyses were performed using MINTAB®, release 14.1 (Minitab Inc., 2003). The extra sum of squares analyses were calculated using Microsoft Excel, with MINTAB® sum of squares input.

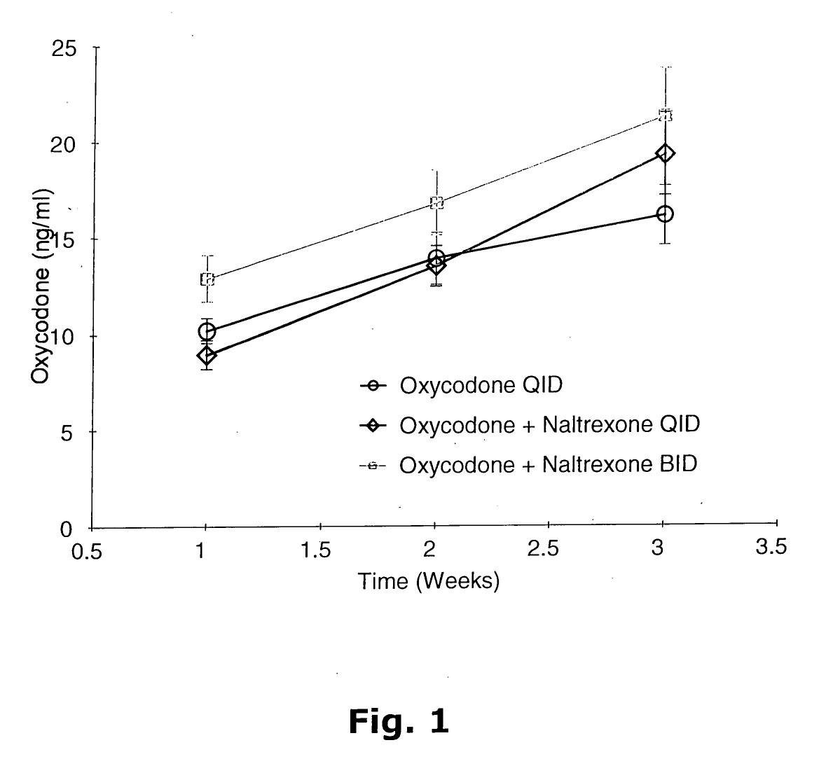

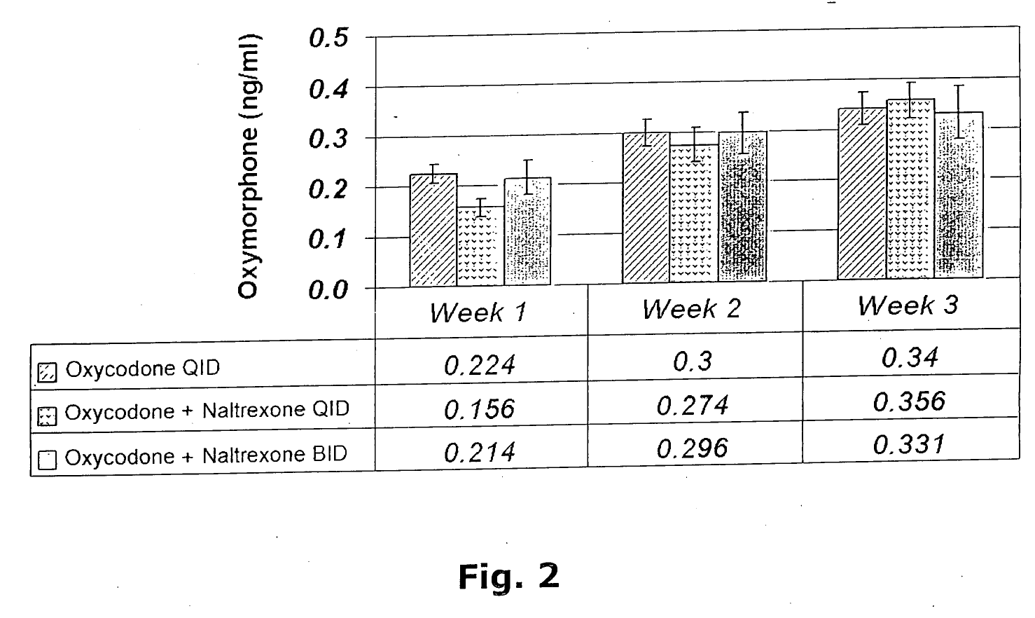

As shown in FIGS. 1 and 2, the mean plasma concentrations of oxycodone and oxymorphone from each drug increased linearly with increasing dose levels over the course of the study. Oxycodone dose levels increase during the course of the study; the oxycodone dose per dose of the combination drug of oxycodone and naltrexone BID was 5 mg (days 1-3), 10 mg (days 4-8), 15 mg (week 2) and 20 mg (week 3). The total daily dose of oxycodone was equal in active treatment arms; i.e., the oxycodone dose in individual doses of the combination drug of oxycodone and naltrexone QID or oxycodone QID was half that of the combination drug of oxycodone and naltrexone BID. The relationship between the oxycodone plasma concentrations and the amount of oxycodone in the dosage form was estimated as: plasma concentration of oxycodone (ng/mL)=2.28+0.4223(dose of oxycodone, mg/day). The slope was statistically significant (p<0.001, t-test) and slopes did not differ significantly among treatments (p=0.258, ANOVA). The test for deviation from linearity was not statistically significant (p=0.787, t-test). The relationship between the oxymorphone plasma concentrations and the amount of oxycodone in the dosage form was estimated to be: plasma concentration of oxymorphone (ng/mL)=0.0607+0.007153(dose of oxycodone, mg/day). The slope was statistically significant (p<0.001, t-test) and the slopes did not differ significantly among treatments (p=0.163, ANOVA). The outcome for the test for deviation from linearity was (p=0.056, t-test). The linear equation coefficients were obtained by averaging patient-specific slopes and intercepts obtained by within-patient least squares regression.

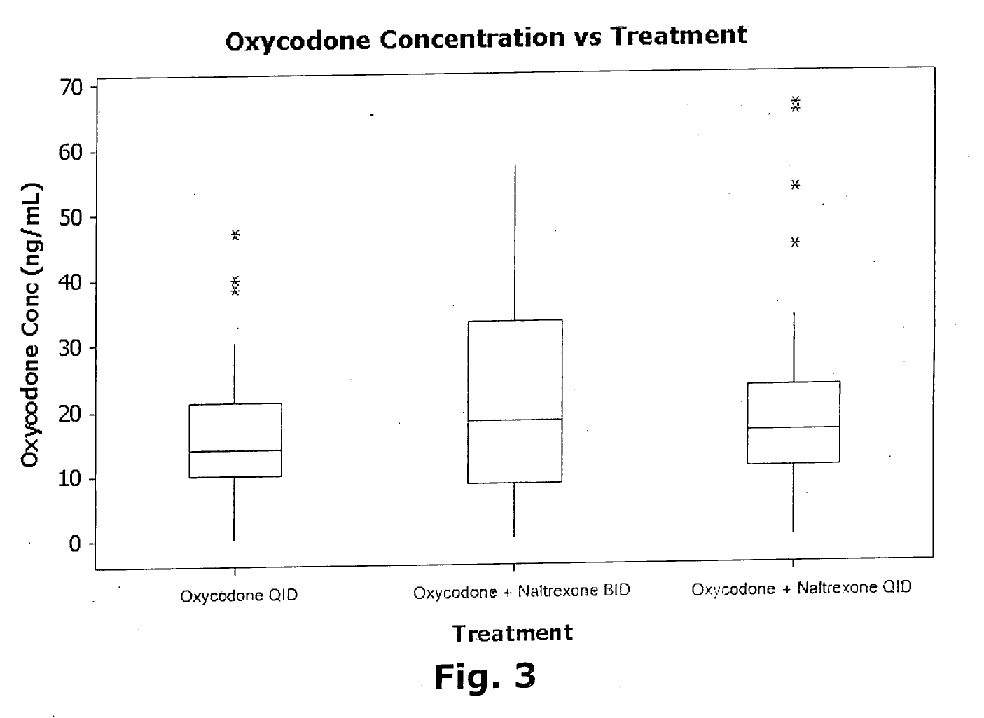

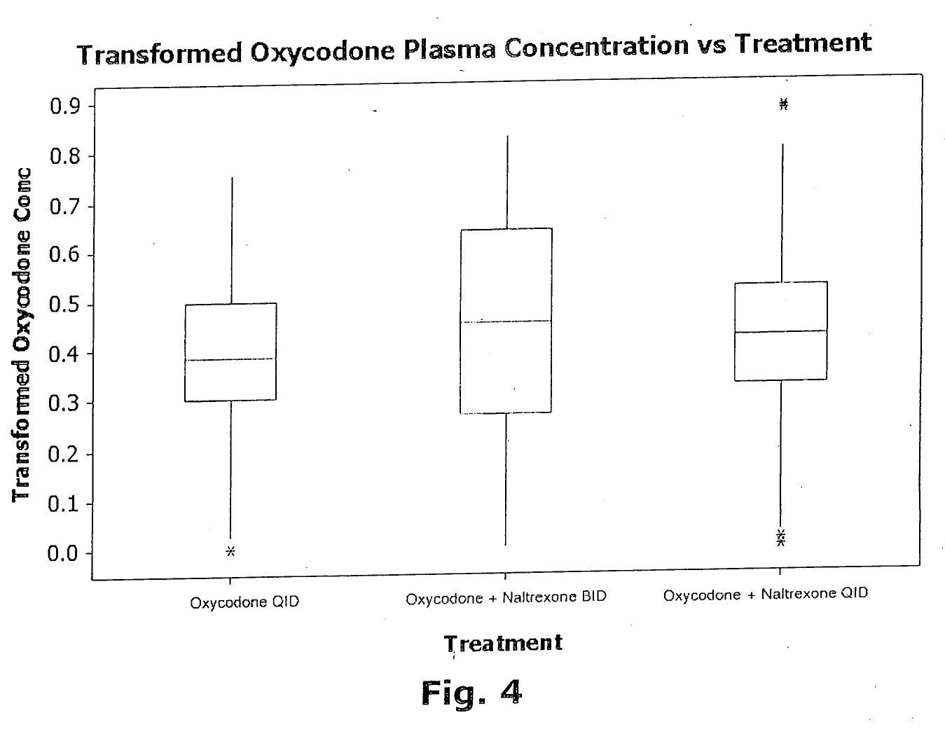

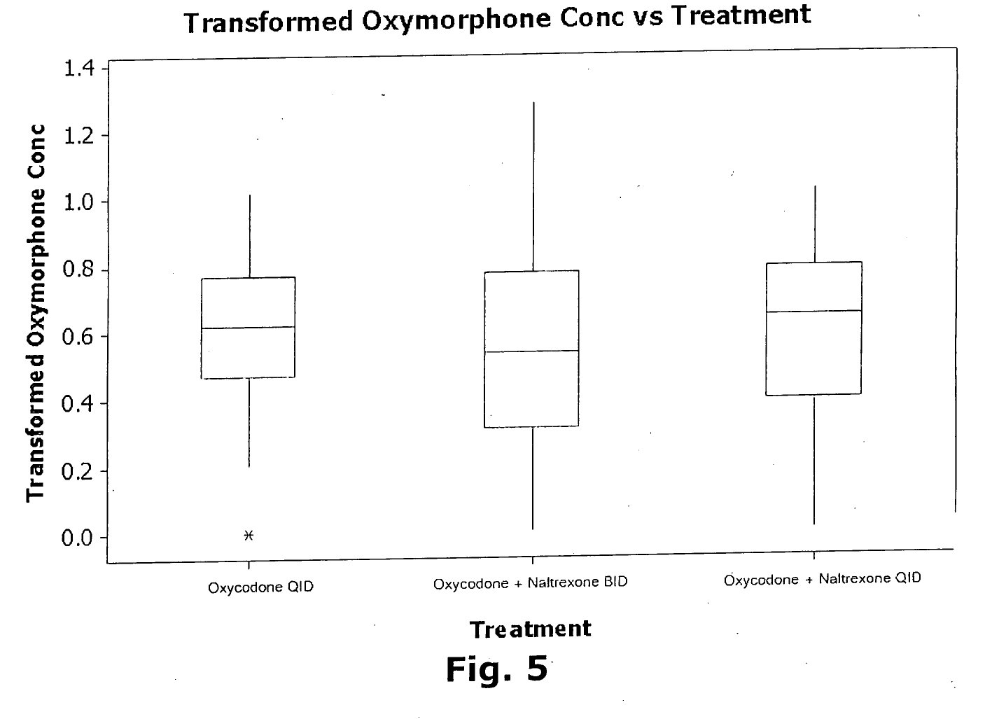

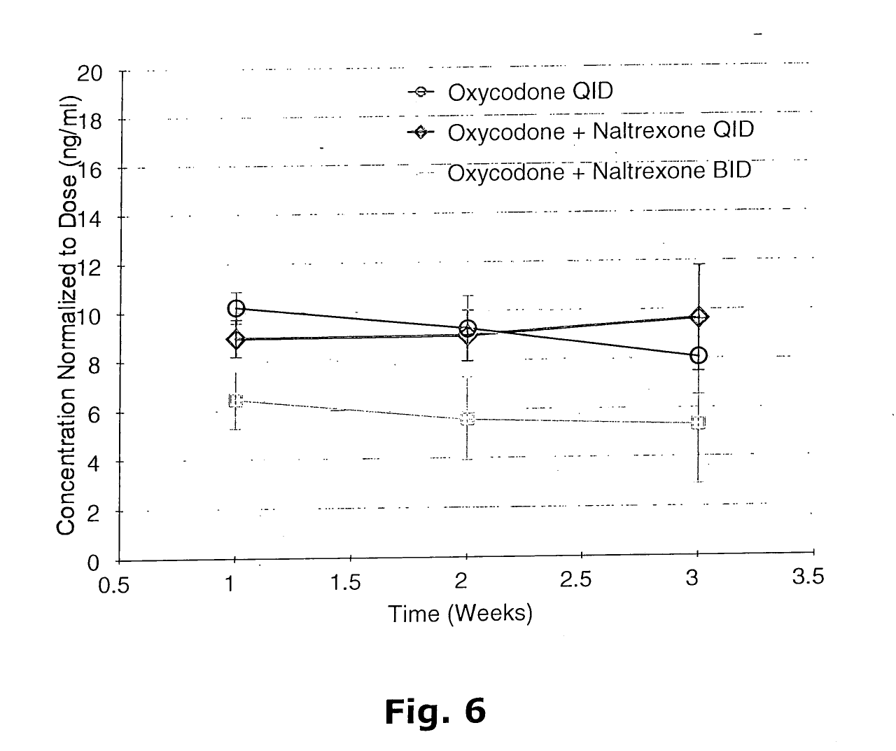

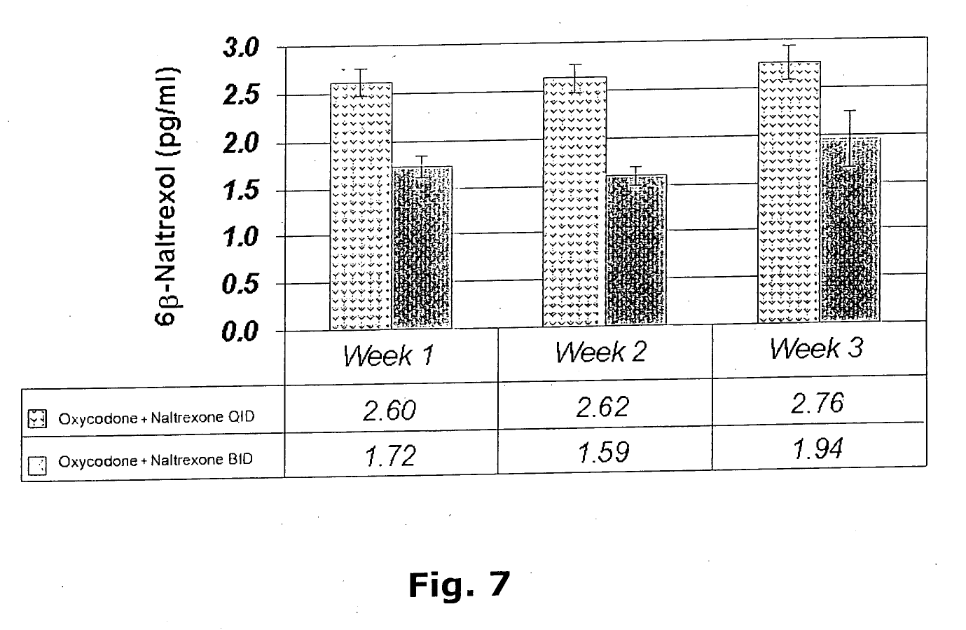

FIG. 3 shows the box-and-whisker plots of the plasma concentrations for oxycodone for each treatment group. FIG. 4 shows the box-and-whisker plots of the transformed plasma concentrations for oxycodone for each treatment group. The results in FIGS. 3 and 4 show that there was no statistical difference (ANOVA, p=0.492) among active treatment groups for oxycodone plasma concentrations. As shown in FIG. 3, the median plasma concentrations of oxycodone are not different following the final dose for each treatment group (* indicates outlying value). As shown in FIG. 4, the median log-transformed plasma concentrations of oxycodone are not different following the final dose for each treatment group (* indicates outlying value). FIG. 5 shows the box-and-whisker plots of the transformed plasma concentrations of oxymorphone for each treatment group. The median log-transformed plasma concentrations of oxymorphone are not different following the final dose for each treatment arm (* indicates outlying value). As shown in FIG. 6, the plasma concentrations of oxycodone and oxymorphone normalized by dose remained constant throughout the study, irrespective of treatment group, suggesting that steady state is achieved and maintained. As shown in FIG. 7, the plasma concentrations of 6β-naltrexol, the major metabolite of naltrexone and a marker for its concentration, remained constant throughout the study (sampled at the end of each treatment week). Maintenance of constant concentrations of 6β-naltrexol, the major metabolite of naltrexone, suggests that naltrexone reached steady state by the end of Week 1 regardless of a 2-fold difference in dose and dosing frequency between treatments.

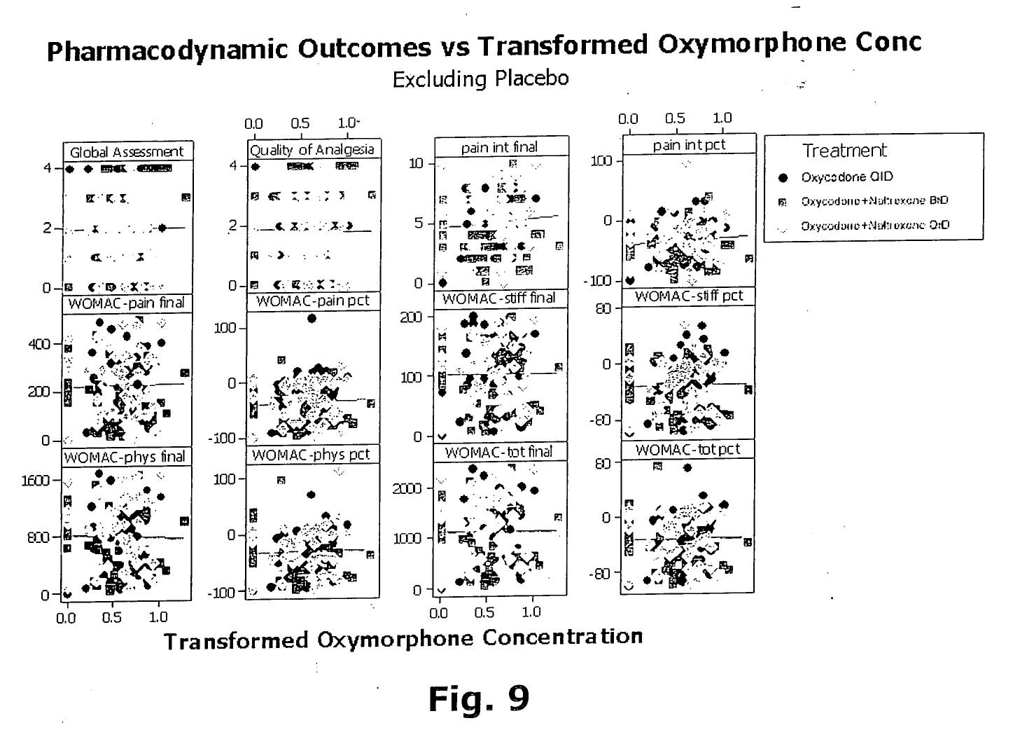

There was no statistically significant difference among treatment groups for any of the correlations between measures of clinical efficacy versus plasma concentrations of oxycodone or oxymorphone (p≧0.193). For the efficacy measurements shown in FIG. 8, the plasma concentrations of oxycodone correlated only with global assessment and quality of analgesia. The lines in each plot panel of FIG. 8 are least square fits. For the efficacy measurements shown in FIG. 9, there was no correlation between the plasma concentrations of oxymorphone and those efficacy measurements. The lines in each plot panel of FIG. 9 are least square fits.

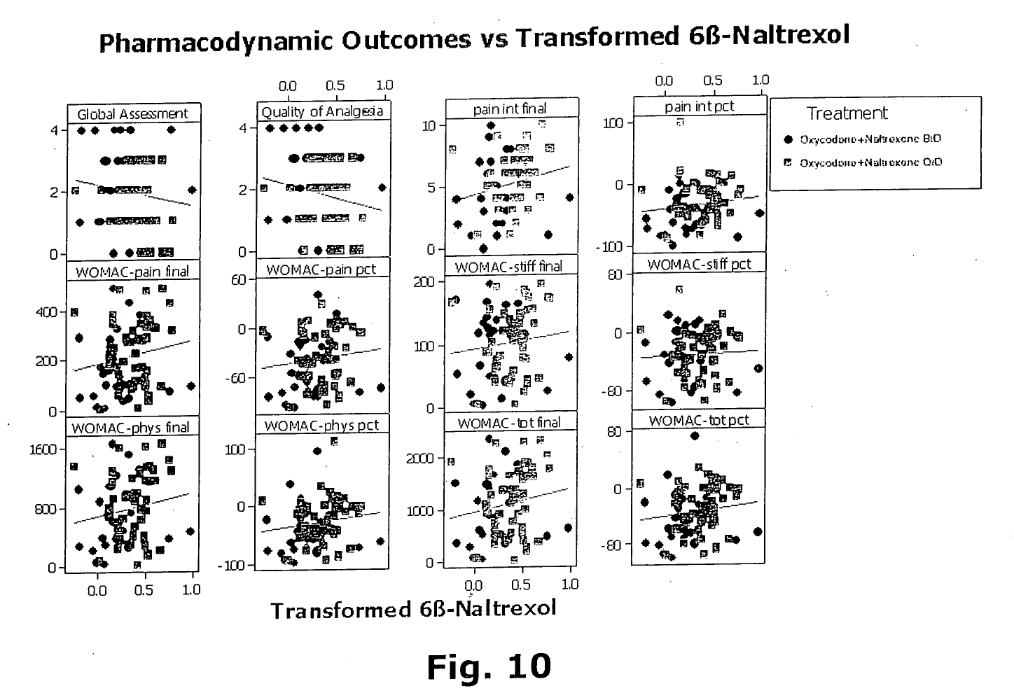

The mean 6β-naltrexol plasma concentration in the BID group is statistically different from that in the QID group (p<0.001, t-test of log-transformed plasma concentrations). There was also a significant difference between the two groups in pain intensity reduction. In addition to these group differences, the relationship between 6β-naltrexol concentrations and clinical outcomes can be observed in the individual patients, with lower plasma concentrations of 6β-naltrexol corresponding to greater clinical efficacy (e.g., pain relief) as shown in FIG. 10. The lines in each plot panel of FIG. 10 are least square fits.

As shown in the above-described figures, the similarity of oxycodone and oxymorphone plasma concentrations after administration of the combination drug of oxycodone and naltrexone BID versus oxycodone QID indicates that the absorption of oxycodone from the combination drug of oxycodone and naltrexone was similar to absorption from oxycodone alone. The plasma concentrations of oxycodone and oxymorphone increased linearly with dose, demonstrating that the exposure to oxycodone from the combination drug of oxycodone and naltrexone is proportional to dose. Maintenance of dose proportionality of oxycodone and oxymorphone throughout the study suggests that steady state was achieved during each dose interval. The consistency of dose proportionality also indicates that the oxycodone elimination half-life did not change during the course of the study. Likewise, the uniform concentrations of 6β-naltrexol, the major metabolite of naltrexone, suggest that naltrexone had attained steady state by the end of Week 1 for both doses and dosing frequencies.

Plasma concentrations of oxycodone and its metabolites, as well as the major metabolite of naltrexone (6β-naltrexol) showed stable pharmacokinetic parameters indicating that the dosage regimens for the combination drug of oxycodone and naltrexone are predictable and easy to manage. Comparisons of the BID and QID dosing regimens for the combination drug of oxycodone and naltrexone showed good correlation between 6β-naltrexol concentration and statistically significant reduction in pain intensity and the percentage change in pain intensity.

The dissimilar clinical response in the presence of similar oxycodone exposures suggests that the naltrexone/6β-naltrexol concentrations are important in determining the threshold for clinical efficacy.

The studies described in this Example show that oxycodone and naltrexone were well-absorbed from the combination drug of oxycodone and naltrexone and that plasma concentrations of oxycodone, oxymorphone and 6β-naltrexol from the combination drug of oxycodone and naltrexone increased directly proportional to the dose and reached steady state over each dosing interval. Clearances and apparent half-lives of oxycodone, oxymorphone and 6β-naltrexol from the combination drug of oxycodone and naltrexone did not change over the course of the study and dose and dosage regimen for the combination drug of oxycodone and naltrexone BID resulted in significantly greater clinical efficacy compared to the QID regimen in reduction in pain intensity or percentage change in pain intensity. Plasma concentrations of oxycodone and oxymorphone did not correlate with greater pain relief and the lowest dose of naltrexone (from the administration of the combination drug of oxycodone and naltrexone BID) utilized in this study resulting in the lowest plasma concentrations of 6β-naltrexol, as a measure of naltrexone plasma concentrations, corresponded to greater pain relief.

### EXAMPLE 3

Data were obtained from a clinical study conducted as described in Examples 1 and 2. Plasma concentrations of the administered drugs and their major metabolites were measured by validated solid phase extraction coupled HPLC-MS/MS. As described in this Example, pharmacokinetic/pharmacodynamic (PK/PD) analyses, including novel applications of modeling analysis, provide novel methods and materials for treating chronic pain, including but not limited to novel dosage forms and methods of administration.

As described in Example 2, the oxycodone and oxymorphone plasma concentration data showed a skewed distribution commonly seen in pharmacokinetic (PK) data. To achieve symmetry, modified log transformations were used as described in Example 2. As noted in Example 2, 6β-naltrexol plasma concentrations did not require transformation to achieve an approximately Gaussian distribution. Table 21 shows the correspondence between the transformed and original scales (where “a” indicates beyond range of observed data).

TABLE 21
Data Transformations of Oxycodone
and Oxymorphone Concentrations

| | Original Oxycodone | Original Oxymorphone |
|---|---|---|
| Transformed | Concentration | Concentration |
| Value | (ng/ml) | (ng/ml) |
| 0.0 | 0.0 | 0.00 |
| 0.1 | 2.6 | 0.03 |
| 0.2 | 5.8 | 0.06 |
| 0.3 | 10.0 | 0.10 |
| 0.4 | 15.1 | 0.15 |
| 0.5 | 21.6 | 0.22 |
| 0.6 | 29.8 | 0.30 |
| 0.7 | 40.1 | 0.40 |
| 0.8 | 53.1 | 0.53 |
| 0.9 | 69.4 | 0.69 |
| 1.0 | A | 0.90 |
| 1.1 | A | 1.16 |
| 1.2 | A | 1.48 |
| 1.3 | A | 1.90 |

Pharmacodynamic outcome measures as described in Example 2 and FIGS. 8-10 were paired with the appropriate analyte plasma concentrations for PK/PD analyses. The plasma concentrations of oxycodone, oxymorphone, naltrexone and 6β naltrexol were measured. It has been observed that plasma concentrations of naltrexone are about one-tenth the plasma concentrations of 6β-naltrexol in the same plasma samples. Accordingly, 6β-naltrexol plasma concentrations are useful as indicators of naltrexone plasma concentrations and to identify preferred plasma concentrations of naltrexone.

Included in the PK/PD analytes were oxycodone and oxymorphone plasma concentrations individually paired with: pain intensity at final visit; pain intensity percent change from baseline at final visit; patient's global assessment at final visit; quality of analgesia at final visit; WOMAC-pain at final visit; WOMAC-pain percent change from baseline at final visit; WOMAC-stiffness at final visit; WOMAC-stiffness percent change from baseline at final visit; WOMAC-physical function at final visit; WOMAC-physical function percent change from baseline at final visit; WOMAC-total score at final visit; and WOMAC-total score percent change from baseline at final visit.

Among the subjects from whom blood was drawn for determination of plasma concentrations, the times from the last dose of the study drug administered to the blood draw were recorded. Times are centered on the hour (e.g., hour 4 covers times from 3.5 up to but excluding 4.5 hours). Table 22 summarizes the times from last dose to blood draw by treatment group for those subjects with plasma concentration data. There was no significant difference among the three treatment groups in time from last dose to blood draw.

TABLE 22
Number of Patients with Plasma Concentration
Data by Hour and Treatment

| | | Oxycodone | Oxycodone + | Oxycodone + |
|---|---|---|---|---|
| Hour | Total | QID | Naltrexone BID | Naltrexone QID |
| 1 | 19 | 7 | 5 | 7 |
| 2 | 31 | 7 | 8 | 16 |
| 3 | 29 | 7 | 13 | 9 |
| 4 | 24 | 11 | 6 | 7 |
| 5 | 15 | 6 | 5 | 4 |
| 6 | 8 | 4 | 2 | 2 |
| >=6.5 | 10 | 3 | 5 | 2 |
| All | 136 | 45 | 44 | 47 |

As described in Example 2, for the analysis of linearity and dose proportionality, linear equation coefficients were obtained by averaging patient-specific slopes and intercepts obtained by within-patient least squares regression. This was done to account for the correlation among the repeated measurements due to the patient's contributing data at each dose level. The resulting slopes among treatment groups were compared by one-way analysis of variance (ANOVA), and a one-sample t-test assessed the common slope's difference from zero. A measure of deviation from linearity was constructed as the difference between the concentration at the middle dose versus the average of the concentrations at the lower and higher doses. Due to equal spacing of doses, this measure has expectation zero under the hypothesis of linearity. As above, ANOVA and t-tests were used to assess linearity.

As also described in Example 2, the relationship between oxycodone plasma concentration and various outcome measures were assessed by Pearson correlation coefficients and associated p-values. For these analyses, the plasma concentration data were log-transformed in order to achieve approximately Gaussian distributions. Oxycodone plasma concentrations (ignoring time of blood draw) in active treatment arms were compared by one-way ANOVA. Regression analyses combined with F-tests on the extra sums of squares assessed whether profiles and correlations differed among active treatment arms. P-values were computed without adjustment for multiple testing. Similar analyses were conducted on the oxymorphone plasma concentrations.

A Kruskal-Wallis test was used to compare active treatment arms with respect to time from last dose to blood draw. The main PK assessment used linear regression analysis to fit the time-concentration profiles. One-way analysis of variance (ANOVA) was used to compare active treatment arms with respect to average oxycodone and oxymorphone plasma concentration (ignoring time of blood draw). Pearson correlation coefficients and associated p-values were used to describe the relationship between plasma concentration versus the outcome measures. Regression analyses combined with F-tests on the extra sums of squares were used to assess whether the time-concentration profiles and correlations differed among the three active treatment arms. P-values were computed and reported without adjustment for multiple testing. As described in Example 2, statistical analyses except extra sum of squares analyses were performed using MINITAB®, release 14.1. The extra sum of squares analyses were calculated using Microsoft Excel, with Minitab sums of squares as input.

Statistically significant correlations (r=0.21, p=0.005) were observed between transformed oxycodone concentration and each of the measures Patient's Global Assessment and Quality of Analgesia (which are pharmacodynamic data), at the final visit. Correlations for the other outcome measures were close to zero and not statistically significant. The calculation of these correlation coefficients included placebo data with imputed oxycodone concentrations of 0.0. In an analysis that excluded the placebo patients, the correlations were smaller and less significant. Table 23 lists the correlation coefficients. There was no statistically significant difference among treatment arms in these plasma concentration and measures of efficacy relationships.

TABLE 23
Correlation Coefficients vs Transformed Oxycodone Concentration

| | | | Oxycodone + | Oxycodone + | |
|---|---|---|---|---|---|
| Pharmacodynamic outcome | All | Oxycodone | Naltrexone | Naltrexone | All except |
| measure¹ | Patients | QID | BID | QID | placebo |
| Pain Intensity at final visit | −0.084 | −0.014 | 0.143 | −0.057 | 0.010 |
| | 0.263 | 0.927 | 0.353 | 0.703 | 0.912 |
| Paint intensity percent | −0.102 | −0.085 | 0.080 | −0.084 | −0.044 |
| change | 0.176 | 0.579 | 0.605 | 0.575 | 0.612 |
| Global assessment at final | 0.209 | 0.174 | 0.055 | 0.147 | 0.124 |
| visit | 0.005 | 0.254 | 0.722 | 0.323 | 0.150 |
| Quality of analgesia at final | 0.211 | 0.207 | −0.009 | 0.145 | 0.114 |
| visit | 0.005 | 0.172 | 0.954 | 0.332 | 0.185 |
| WOMAC-Pain at final visit | −0.070 | −0.049 | 0.192 | −0.230 | −0.044 |
| | 0.356 | 0.748 | 0.213 | 0.120 | 0.611 |
| WOMAC-Pain percent | −0.057 | −0.040 | 0.196 | −0.237 | −0.044 |
| change | 0.451 | 0.793 | 0.203 | 0.109 | 0.612 |
| WOMAC-Stiffness at final | −0.070 | −0.030 | 0.088 | −0.151 | −0.038 |
| visit | 0.352 | 0.844 | 0.573 | 0.312 | 0.665 |
| WOMAC-Stiffness percent | −0.042 | 0.102 | 0.078 | −0.208 | −0.015 |
| change | 0.576 | 0.506 | 0.618 | 0.162 | 0.865 |
| WOMAC-Physical | −0.071 | −0.036 | 0.123 | −0.216 | −0.055 |
| functioning at final visit | 0.345 | 0.815 | 0.433 | 0.145 | 0.529 |
| WOMAC-Physical | −0.060 | 0.015 | 0.089 | −0.211 | −0.044 |
| functioning percent change | 0.426 | 0.924 | 0.570 | 0.155 | 0.615 |
| WOMAC-Total score at | −0.072 | −0.038 | 0.137 | −0.215 | −0.052 |
| final visit | 0.343 | 0.802 | 0.381 | 0.147 | 0.551 |
| WOMAC-Total score | −0.062 | 0.010 | 0.125 | −0.249 | −0.044 |
| percent change | 0.417 | 0.948 | 0.425 | 0.091 | 0.614 |

¹For each pharmacodynamic outcome measure, the first row of data displays correlation coefficients, and the second row displays corresponding p-values.

The concentration time course of oxymorphone was well modeled by a straight line. Separate regression lines were also fit for each treatment group. Observed differences in slope are not statistically significant. Overall, ignoring time of blood draw, there was no statistically significant difference among active treatment arms in transformed oxymorphone plasma concentration. None of the correlations between oxymorphone and outcome measures was statistically significant, as shown in Table 24. There was no statistically significant difference among treatment arms in these PK/PD relationships.

TABLE 24
Correlation Coefficients vs Transformed Oxymorphone Concentration

| | | | Oxycodone + | Oxycodone + | |
|---|---|---|---|---|---|
| Pharmacodynamic outcome | All | Oxycodone | Naltrexone | Naltrexone | All except |
| measure¹ | Patients | QID | BID | QID | placebo |
| Pain Intensity at final visit | −0.044 | 0.293 | −0.095 | 0.021 | 0.079 |
| | 0.560 | 0.051 | 0.540 | 0.889 | 0.359 |
| Paint intensity percent change | −0.028 | 0.228 | −0.099 | 0.061 | 0.076 |
| | 0.709 | 0.131 | 0.521 | 0.685 | 0.379 |
| Global assessment at final visit | 0.140 | −0.126 | 0.297 | −0.171 | 0.011 |
| | 0.063 | 0.410 | 0.051 | 0.251 | 0.898 |
| Quality of analgesia at final visit | 0.130 | −0.126 | 0.230 | −0.144 | −0.015 |
| | 0.085 | 0.409 | 0.134 | 0.333 | 0.863 |
| WOMAC-Pain at final visit | −0.028 | 0.148 | −0.041 | −0.074 | 0.026 |
| | 0.711 | 0.332 | 0.789 | 0.623 | 0.766 |
| WOMAC-Pain percent change | 0.007 | 0.153 | −0.036 | −0.002 | 0.060 |
| | 0.930 | 0.317 | 0.817 | 0.991 | 0.491 |
| WOMAC-Stiffness at final visit | −0.045 | 0.155 | −0.141 | −0.018 | 0.005 |
| | 0.553 | 0.308 | 0.366 | 0.905 | 0.953 |
| WOMAC-Stiffness percent | −0.026 | 0.215 | −0.170 | 0.002 | 0.012 |
| change | 0.730 | 0.157 | 0.275 | 0.989 | 0.891 |
| WOMAC-Physical functioning at | −0.045 | 0.139 | −0.146 | −0.051 | −0.011 |
| final visit | 0.548 | 0.364 | 0.349 | 0.734 | 0.896 |
| WOMAC-Physical functioning | −0.017 | 0.184 | −0.221 | 0.139 | 0.026 |
| percent change | 0.818 | 0.227 | 0.154 | 0.350 | 0.765 |
| WOMAC-Total score at final visit | −0.043 | 0.143 | −0.129 | −0.053 | −0.003 |
| | 0.574 | 0.347 | 0.411 | 0.723 | 0.973 |
| WOMAC-Total score percent | −0.021 | 0.184 | −0.174 | 0.059 | 0.023 |
| change | 0.782 | 0.227 | 0.264 | 0.692 | 0.794 |

¹For each pharmacodynamic outcome measure, the first row of data displays correlation coefficients and the second row displays corresponding p-values.

Pharmacokinetic and pharmacodynamic data (e.g., percentage change in pain intensity) associated with the administration of oxycodone and naltrexone in clinical studies as described above were evaluated to identify desirable parameters involving dosage forms comprising naltrexone. Table 25 shows 6β-naltrexol plasma concentrations from the randomly selected samples for the subjects receiving oxycodone and naltrexone. Table 25 also shows pain intensity measurements for those subjects, including pain intensity baseline, final pain intensity, and the percent change in pain intensity. As discussed in more detail below, the percent change in pain intensity was the drug effect used in a modeling analysis of plasma concentration vs. drug effect.

TABLE 25
PK/PD Data For Combination Drug of Oxycodone
and Naltrexone In QID and BID Dosing Regimens

| | | | Time from | Pain Int | Pain Int | Pain Int | |
|---|---|---|---|---|---|---|---|
| Subject | Treatment | Sex | Last Dose | Base | Final | Percent | 6βNaltrexol |
| 004-0530 | QID | F | 2.22 | 9 | 7 | −22.222 | 3.29 |
| 004-0560 | BID | F | 2.67 | 8 | 1 | −87.5 | 5.67 |
| 004-0741 | QID | F | 4.25 | 10 | 8 | −20 | 3.25 |
| 006-0015 | QID | M | 4.2 | 8 | 7 | −12.5 | 2.47 |
| 006-0554 | BID | F | 2.42 | 7 | 1 | −85.714 | 0.89 |
| 006-0592 | QID | F | 0.5 | 8 | 4 | −50 | 4.69 |
| 006-0700 | QID | F | 3.05 | 9 | 6 | −33.333 | 3.16 |
| 006-0705 | BID | F | 2.52 | 9 | 8 | −11.111 | 2.24 |
| 006-0724 | BID | F | 2.53 | 8 | 6 | −25 | |
| 006-0727 | QID | F | 4.33 | 9 | 5 | −44.444 | 2.95 |
| 007-0011 | BID | M | 2.25 | 7 | 2 | −71.429 | 1.57 |
| 007-0045 | BID | M | 1.5 | 8 | 4 | −50 | |
| 007-0088 | QID | M | 3.92 | 7 | 7 | 0 | 3.58 |
| 007-0097 | BID | M | 2.33 | 6 | 2 | −66.667 | |
| 007-0520 | QID | F | 1.83 | 7 | 6 | −14.288 | |
| 007-0650 | BID | F | 0.97 | 9 | 4 | −55.556 | 0.61 |
| 009-0003 | QID | M | 1.92 | 9 | 8 | −11.111 | 0.56 |
| 009-0022 | BID | M | 2.08 | 6 | 1 | −83.333 | 1.64 |
| 009-0025 | QID | M | 0.67 | 9 | 6 | −33.333 | 4.07 |
| 009-0049 | QID | M | 0.67 | 9 | 8 | −11.111 | 5.98 |
| 009-0058 | QID | M | 1.33 | 10 | 8 | −20 | 5.63 |
| 009-0612 | BID | F | 4.58 | 8 | 3 | −62.5 | 1.2 |
| 010-0030 | QID | M | 1.83 | 7 | 8 | 14.286 | 2.86 |
| 010-0033 | BID | M | 3.58 | 7 | 4 | −42.857 | 1.6 |
| 010-0501 | BID | F | 26.17 | 6 | 5 | −16.667 | 1.56 |
| 012-0026 | QID | M | 5.73 | 3 | 6 | 100 | 1.39 |
| 012-0029 | BID | M | 5.25 | 7 | 7 | 0 | 1.41 |
| 012-0031 | QID | M | 2.78 | 7 | 6 | −14.286 | 3.18 |
| 012-0641 | BID | F | 2 | 10 | 4 | −60 | |
| 013-0001 | BID | M | 5.08 | 6 | 1 | −83.333 | 0.97 |
| 013-0008 | QID | M | 1.75 | 7 | 1 | −85.714 | 2.24 |
| 013-0109 | QID | M | 3.25 | 7 | 6 | −14.286 | 2.45 |
| 013-0565 | QID | F | 1.67 | 7 | 4 | −42.857 | 1.61 |
| 015-0095 | BID | M | 3 | 10 | 5 | −50 | 1.57 |
| 015-0572 | QID | F | 2.83 | 7 | 1 | −85.714 | |
| 015-0733 | BID | F | 2.5 | 7 | 4 | −42.857 | 2.09 |
| 016-0108 | BID | M | 3.92 | 8 | 2 | −75 | 1.86 |
| 016-0563 | BID | F | 4.42 | 8 | 4 | −50 | 9.54 |
| 016-0590 | BID | F | 2.83 | 10 | 4 | −60 | |
| 016-0620 | QID | F | 1.5 | 7 | 3 | −57.143 | 3.49 |
| 018-0047 | BID | M | 3 | 5 | 3 | −40 | |
| 018-0064 | QID | M | 1.33 | 5 | 2 | −60 | 2.63 |
| 018-0068 | QID | M | 2.28 | 7 | 6 | −14.286 | 3.13 |
| 018-0611 | QID | F | 2 | 9 | 1 | −88.889 | 1.05 |
| 018-0660 | BID | F | 3.42 | 10 | 6 | −40 | 2.75 |
| 019-0534 | BID | F | 1 | 7 | 3 | −57.143 | |
| 020-0021 | QID | M | 3.08 | 7 | 7 | 0 | 1.96 |
| 020-0537 | BID | F | 3.17 | 9 | 3 | −66.667 | 2.09 |
| 020-0538 | QID | F | 2.5 | 8 | 7 | −12.5 | 2.66 |
| 022-0053 | BID | M | 0.75 | 8 | 5 | −37.5 | 1.29 |
| 022-0511 | QID | F | 2 | 7 | 6 | −14.286 | 2.31 |
| 022-0585 | QID | F | 0.75 | 10 | 5 | −50 | 1.33 |
| 022-0725 | QID | F | 3 | 9 | 10 | 11.111 | |
| 022-0726 | BID | F | 2.5 | 10 | 7 | −30 | 3.25 |
| 022-0729 | BID | F | 3.75 | 7 | 6 | −14.286 | |
| 023-0079 | BID | M | 4.25 | 8 | 9 | 12.5 | 1.33 |
| 023-0101 | QID | M | 6.17 | 8 | 5 | −37.5 | 1.47 |
| 025-0564 | BID | F | 3.67 | 10 | 10 | 0 | 1.4 |
| 026-0004 | BID | M | 3.33 | 6 | 5 | −16.667 | 1.64 |
| 026-0036 | QID | M | 4.6 | 6 | 6 | 0 | 3.92 |
| 026-0075 | BID | M | 5.23 | 7 | 0 | −100 | 1.17 |
| 026-0076 | QID | M | 2.2 | 5 | 4 | −20 | 2.9 |
| 026-0604 | BID | F | 7.52 | 8 | 5 | −37.5 | 1.41 |
| 026-0656 | BID | F | 1.37 | 8 | 3 | −62.5 | |
| 029-0107 | QID | M | 2 | 7 | 4 | −42.857 | 1.6 |
| 029-0528 | BID | F | 6.58 | 9 | 2 | −77.778 | |
| 029-0584 | QID | F | 3.92 | 10 | 6 | −40 | 2.01 |
| 031-0634 | QID | F | 3 | 8 | 6 | −25 | 3.53 |
| 032-0055 | BID | M | 2.67 | 7 | 4 | −42.857 | 1.1 |
| 032-0086 | QID | M | 4 | 8 | 5 | −37.5 | 1.32 |
| 032-0543 | QID | F | 3.42 | 8 | 9 | 12.5 | 3.35 |
| 032-0709 | QID | F | 2.25 | 9 | 5 | −44.444 | 3.38 |
| 033-0012 | QID | M | 6.92 | 7 | 4 | −42.857 | 1.81 |
| 033-0546 | QID | F | 2.42 | 8 | 5 | −37.5 | 3.23 |
| 035-0063 | BID | M | 8 | 7 | 2 | −71.429 | 0.63 |
| 035-0072 | BID | M | 0.92 | 7 | 4 | −42.857 | |
| 035-0594 | BID | F | 2.13 | 9 | 3 | −66.667 | 2.33 |
| 037-0052 | QID | M | 1.17 | 8 | 4 | −50 | 2.5 |
| 037-0074 | QID | M | 7.08 | 5 | 6 | 20 | 1.4 |
| 037-0615 | QID | F | 3.75 | 10 | 9 | −10 | 1.65 |
| 037-0711 | QID | F | 4.75 | 10 | 3 | −70 | 3.48 |
| 038-0046 | QID | M | 5.17 | 7 | 3 | −57.143 | 1.72 |
| 040-0644 | BID | F | 4.75 | 8 | 7 | −12.5 | 1.06 |
| 041-0069 | BID | M | 3.08 | 8 | 6 | −25 | 1.3 |
| 041-0098 | QID | M | 2.35 | 8 | 3 | −62.5 | 2.4 |
| 041-0649 | QID | F | 2.42 | 9 | 10 | 11.111 | 4.75 |
| 042-0105 | BID | M | 9.17 | 8 | 8 | 0 | 2.09 |
| 043-0062 | BID | M | 6 | 8 | 2 | −75 | |
| 043-0081 | BID | M | 6 | 6 | 2 | −66.667 | |

As described in Example 2, the mean 6β-naltrexol plasma concentration in the oxycodone and naltrexone BID group was statistically different from that in the oxycodone and naltrexone QID group (p<0.001). There was also a significant difference between the BID group and the QID group in pain intensity reduction, with the BID group experiencing a significant reduction in pain intensity. It was unexpected that the QID group and BID group would differ in this manner. The plasma concentrations of 6β-naltrexol appear to be at steady state at the conclusion of each dosing interval. (See FIG. 7). Therefore, in addition to the above dose group differences, the plasma concentration-effect relationship between 6β-naltrexol concentrations and clinical outcomes (effects) can be observed, with lower steady state plasma concentrations of 6β-naltrexol corresponding to greater clinical efficacy (e.g., percent change in pain intensity).

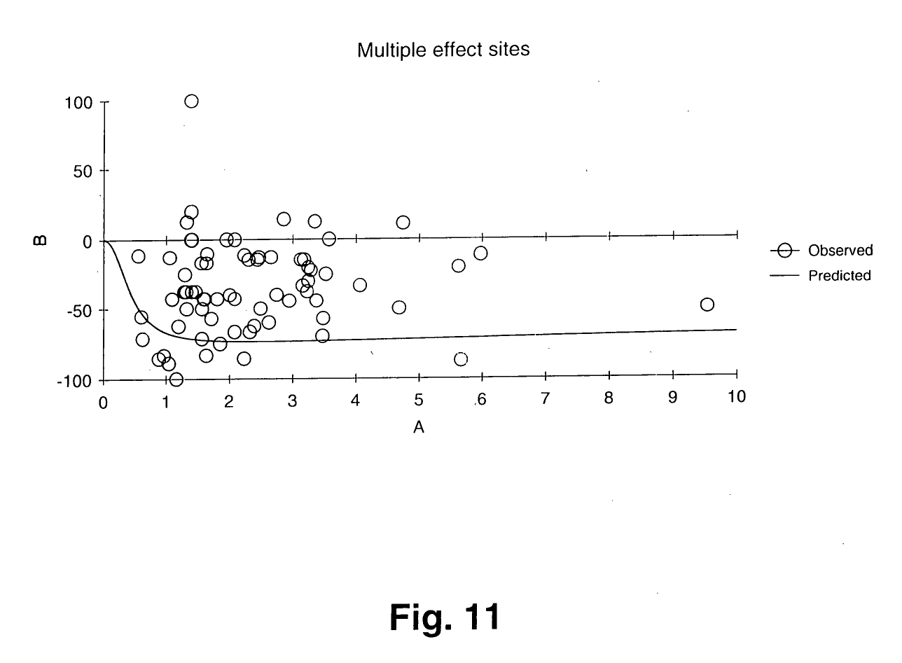

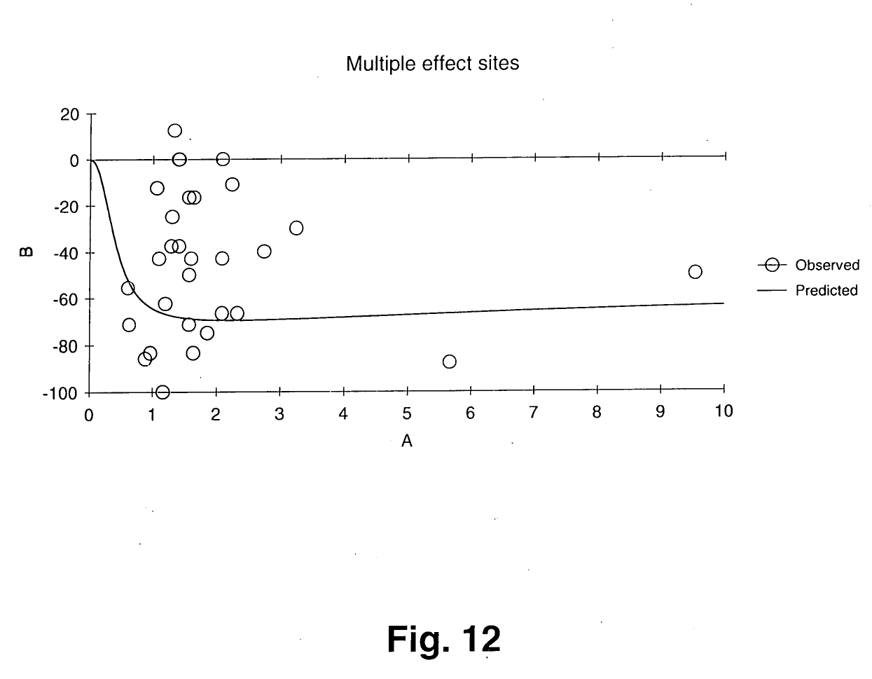

FIGS. 11 and 12 illustrate this plasma concentration-effect relationship. FIG. 11 plots the percent change in pain intensity reported by the subjects in Table 25 (y-axis) vs. 6β-naltrexol plasma concentrations measured for those subjects α-axis). FIG. 11 includes data from subjects receiving the BID dosing regimen of the combination drug and subjects receiving the QID dosing regimen of the combination drug. The data as plotted in FIG. 11 describe a U shaped plasma concentration-effect relationship. FIG. 12 plots the percent change in pain intensity reported by the subjects receiving the BID dosing regimen of the combination drug vs. 6β-naltrexol plasma concentrations measured for those subjects.

For the first time, the plasma concentration-effect relationship of low dose of an opioid antagonist when administered with an opioid agonist has been represented by the Emax composite model:
E[Emax1(Cpⁿ¹)/EC51ⁿ¹+Cpⁿ¹]+[Emax2(Cpⁿ²)/EC52ⁿ²+Cpⁿ²]
where the respective Emax values represent maximum effect for a given drug; EC51 and EC52 represent the potencies, for the drug notated as either 1 or 2, respectively (in other words, EC51 is not the concentration having 51% of the maximal effect, but rather EC51 is the concentration having a particular potency (e.g. 50% of the maximal effect for Effect No. 1); the respective values for C are the concentrations of drugs notated as 1 or 2, and the values of n₁ and n₂ that correspond to the sigmoidicity factors that are associated with particular EC values. In the Emax composite model, “+” is used to indicate absolute values; sometimes it is shown as a “−” which reflects a negative second term.

The Emax composite model is a recognized composite model for PK/PD data analysis set forth, for example, in Gabrielsson et al., PHARMACOKINETIC/PHARMACODYNAMIC DATA ANALYSIS: CONCEPTS AND APPLICATIONS, pp. 191-193 and 801-808 (2000), and the computer command files provided with the reference and described, including with examples of the computer printouts on pages 801-808, all of which is incorporated by reference herein. However, it is believed that the Emax composite model has not previously been utilized for the analysis of PK data from administering low doses of opioid antagonists such as naltrexone for enhancing the potency of opioid agonists such as oxycodone, as described herein. From the plasma concentration-effect data obtained in this Example, it is contemplated that the opioid antagonist, at lower plasma concentrations, is impacting the total effect (percent change in pain intensity), primarily as described by the terms of the equation denoted with a 2.

The recognition of the applicability and utility of a composite model as shown above enables the selection of preferred and/or suitable ranges for the combined use of an opioid antagonist with an opioid agonist as described herein. The composite model provides the relative contribution of an opioid antagonist with respect to enhancing pain relief, for example, as measured by a reduction in pain intensity. The effective percentage decrease in pain intensity, E, has been found to be described by a relatively wide scope of preferred plasma concentrations by the Emax composite model, as shown in the data and Figures described herein.

The plasma concentration-effect data were fit to the Emax composite model using the software program WinNonlin® (commercially available from Pharsight Corporation of Mountain View, Calif.) and the command files developed by Gabrielsson et al. The plasma concentration-effect data represented as circles in FIG. 11 were evaluated mathematically and the plasma concentration-effect curve shown in FIG. 11 was determined by the program and command files. Similarly, the program and command files were used to determine the plasma concentration-effect curve shown in FIG. 12 based on the data represented as circles in FIG. 12.

The computer output (printout) of this process included EC51 and EC52 parameters, as well as parameters reflecting statistical evaluation of the data, such as coefficient of variation (CV %). A variety of values, for example EC20 and EC90 (the concentrations at which 20% and 90%, respectively, of the maximum effect are obtained), may also be determined using the output from the WinNonlin® program and Gabrielsson et al. command files (or similar programs and command files). Other values, for example ECO and EC100 and all values in between, also may be determined graphically and/or using the values of N1 and N2 that correspond to the sigmoidicity factors.

Table 26 shows parameters based on the curve shown in FIG. 11. These parameters are based on the data for 6β-naltrexol plasma concentrations and reduction in pain from baseline pain intensity to final pain intensity from all subjects for whom plasma concentration data was obtained as described herein. These parameters are based on data from subjects receiving the BID dosing regimens and subjects receiving the QID dosing regimen. Estimate refers to the value estimated by the WinNonlin® program and command file for relating plasma concentrations to the pharmacodynamic effects such as percent reduction in pain intensity. Convergence of the model was easily achieved and the power of the condition number was acceptable.

TABLE 26
Parameters based on Total (BID and QID) data

| Parameter | Estimate | |
|---|---|---|
| Emax1 | 26.8 | units |
| EC51 | 14.4 | pg/ml |
| N1 (sigmoidicity factor) | 0.907 | units/pg/ml |
| Emax2 | 79.7 | units |
| EC52 | 0.439 | pg/ml |
| N2 (sigmoidicity factor) | 2.28 | units/pg/ml |

Table 27 shows parameters based on the curve shown in FIG. 12. These parameters are based on the data (6β-naltrexol plasma concentrations and percent reduction in pain intensity) for subjects receiving BID dosing regimens. Convergence of the model was easily achieved and the power of the condition number was acceptable.

TABLE 27
Parameters based on BID data

| Parameter | Estimate | |
|---|---|---|
| Emax1 | 27.0 | units |
| EC51 | 14.0 | pg/ml |
| N1 (sigmoidicity factor) | 0.941 | units/pg/ml |
| Emax2 | 76.3 | units |
| EC52 | 0.422 | pg/ml |
| N2 (sigmoidicity factor) | 2.16 | units/pg/ml |

As mentioned above, the BID dosing regimen of the combination drug comprising naltrexone and oxycodone resulted in statistically significant decreases in pain intensity. The Emax composite model provided the value of a EC52 plasma concentration of 6β-naltrexol based on that BID dosing regimen. Substantially the same EC52 result was obtained from the analysis of the total data set (comprising data from both the BID and QID dosing regimens). The fact that substantially the same EC52 result was obtained from the different data sets supports the strength of the Emax composite model for analysis of the data. It also supports the use of the Emax composite model in order to select desirable doses of naltrexone (or another opioid antagonist) in combination with oxycodone.

Tables 26 and 27 illustrate the use of the total set of clinical data and the subset associated with positive clinical results in the same Emax composite model to provide two sets of parameters. Either or both of the two sets of parameters can be used to identify plasma concentrations having a probability of attaining a desired reduction of pain intensity or other efficacy outcome (e.g., pharmacodynamic outcome) as described herein. From the plasma concentration-effect data and the Emax composite model, one can better assess what plasma concentrations of 6β-naltrexol provide desired reduction in pain intensity and, more generally, better pain treatment. Based on plasma concentration data (e.g., as shown in Table 25), presently preferred dosage forms for oral administration to a human subject comprise a dose amount of opioid antagonist that is based on a selected plasma concentration. Thus, naltrexone and/or 6β-naltrexol may be used to titrate a subject to the appropriate dose for that subject thus providing a convenient means for individualized dosing.

The Emax composite model can facilitate dose titration for a human subject. Dose titration refers to the process of employing different doses (usually escalating doses) in a subject until a dose effective to achieve a desired clinical outcome is found. Dose titration for the administration of an opioid antagonist and/or an opioid agonist according to the present invention may be facilitated by using plasma concentrations of 6β-naltrexol, naltrexone, or another marker of opioid antagonist. Dose titration may also be facilitated by using plasma concentrations of oxycodone, oxymorphone, or another marker of opioid agonist may be used alone or in combination with a marker of opioid antagonist for dose titration.

For dose titration of the administration of an opioid antagonist and an opioid agonist to a human subject, the subject's plasma concentration of 6β-naltrexol, naltrexone or another marker for opioid antagonist is analyzed, and one or more clinical outcomes (such as reduction in pain intensity) for the subject are analyzed. If a desired clinical outcome is not achieved (for example, if pain intensity is not reduced to a desired level), the administration of opioid antagonist and/or opioid agonist to the subject is adjusted. The composite model can be used to facilitate adjusting, or facilitate the decision to adjust, the administration of (a) the opioid antagonist or (b) the opioid agonist or (c) both.

In the present method of titrating a human subject, if a clinical outcome is not at a desired level, the plasma concentration of 6β-naltrexol is analyzed. If the 6β-naltrexol plasma concentration is not at a desired level, then administration of the opioid antagonist to the subject is adjusted. The administration of the opioid antagonist may be adjusted by adjusting the dose amount and/or dosing regimen. However, if the 6β-naltrexol plasma concentration is already at a desired level, yet the clinical outcome is not at a desired level, then the administration of the opioid agonist to the subject is adjusted. The administration of the opioid agonist may be adjusted by adjusting the dose amount and/or dosing regimen.

For example, in a method of titrating the administration of an opioid agonist and an opioid antagonist a human subject to reduce pain intensity in the subject, if the reduction in pain intensity is not at a desired level, the plasma concentration of 6β-naltrexol is analyzed. If the 6β-naltrexol plasma concentration is below a desired level, then administration of the opioid antagonist to the subject is adjusted so that more opioid antagonist is administered to the subject. If the 6β-naltrexol plasma concentration is above a desired level, then administration of the opioid antagonist to the subject is adjusted so that less opioid antagonist is administered to the subject. However, if the 6β-naltrexol plasma concentration is already at a desired level, yet the reduction in pain intensity is not at a desired level, then the administration of the opioid agonist to the subject is adjusted so that more opioid agonist is administered to the subject.

The Emax composite model may be used to identify desired levels of the plasma concentration of opioid antagonist, for example a level indicated by the composite model as having a desired level of efficacy. For example, parameters, including but not limited to EC20, EC50 and EC90, identified by the composite model may be employed to select desirable levels of plasma concentrations of opioid antagonist (as measured directly or via a surrogate marker such as 6β-naltrexol).

Parameters provided by the composite model may be employed to select desirable doses of naltrexone from the plasma concentrations of 6β-naltrexol, based on the foregoing data, parameters and adjustments relating to 6β-naltrexol. As mentioned above, when naltrexone is administered to a human subject, the plasma concentration of 6β-naltrexol is useful as an indicator of the absorption of naltrexone, since 6β-naltrexol is generally present in plasma at concentrations much higher than those of naltrexone due to the rapid metabolism of naltrexone to yield 6β-naltrexol. For example, a 6β-naltrexol plasma concentration of about 0.4 pg/ml indicates a naltrexone plasma concentration of about 0.04 pg/ml in the plasma sample, and where a given 6β-naltrexol plasma concentration is provided herein, a naltrexone plasma concentration of about 1/10 of the given 6β-naltrexol plasma concentration is also contemplated.

The plasma concentration of 6β-naltrexol at steady state is generally proportional to the dose amount of naltrexone in a BID dosing regimen. It has been found that a dose of an opioid antagonist such as naltrexone given as a BID regimen that produces plasma concentrations of free 6β-naltrexol that are related by a proportionality factor to naltrexone correlated for a given dose of an opioid agonist statistically (p<0.001) with percent decreases in pain intensity from base line for moderate to severe pain.

Accordingly, a desirable dose amount of opioid antagonist, and optionally a desirable dose amount of opioid agonist, can be selected based on a steady state plasma concentration that exhibits a desired pharmacodynamic (PD) effect. Exemplary data for plasma concentrations and PD effects are shown in Table 25. Based on the proportional relationship between concentration and dose, a formula for converting between concentration and dose can be established by experimentally determining plasma concentrations that result from known dose amounts. This formula may be used to select dose amounts of opioid antagonists converted from plasma concentrations showing a desired PD effect. Furthermore, the dose of a co-administered opioid agonist may be adjusted, by increasing or decreasing the dose, relative to the opioid antagonist, to further optimize pain relief or other efficacy outcomes as described herein.

This linear relationship is true for the case where the daily dosing regimen results in a steady state plasma concentration. In the present Example, the greatest frequency of obtaining plasma concentrations associated with significant improvement in pain relief as reflected by the percentage change in the pain intensity score was obtained for the dose of naltrexone given BID. Naltrexone at the dose as described herein when given more frequently than BID resulted in a greater proportion of 6β-naltrexol concentrations increasing above those for the BID dosing regimen in a statistically significant (p<0.0001) proportion of the population. Stated differently, the plasma levels of naltrexone, as measured by its major metabolite 6β-natrexol were too high in the OID dosing regimen, thus a statistically significant increase in pain relief with the QID dosing regimen of naltrexone as described herein was not achieved. However, since individual patients in the QID dosing group did not achieve an increase in pain relief as shown in Table 25, a statistically significant increase in pain relief with a similar QID dosage regimen of the opioid antagonist (e.g., naltrexone) may be achieved when the dose of the opioid agonist (e.g., oxycodone) is increased relative to the amount of antagonist.

Parameters, including but not limited to EC20, EC50 and EC90, identified by the composite model may be employed to select desirable amounts of opioid antagonist in various dosage forms. A desired amount of opioid antagonist can be determined from a selected plasma concentration arising from a known amount of opioid antagonist, since the relationship between concentration and dose amount is generally linearly proportional. The plasma concentrations of 6β naltrexol from randomly selected samples from subjects receiving 1 μg of naltrexone and 20 mg of oxycodone in a BID dosing regimen were fit to the Emax composite model. The EC52 of 6β naltrexol in the plasma, as the surrogate marker for the active drug naltrexone in the plasma, corresponding to 1 μg of naltrexone from the BID dosing regimen was computed.

By way of example, but not as a limitation, parameters provided by a composite model are useful for predicting doses from desirable lower levels of plasma concentrations of 6β-naltrexol. More particularly, the EC52 parameter in Table 26 suggests that a 6β-naltrexol plasma concentration of about 0.439 pg/ml or more may be employed to attain better than a 50% reduction in pain intensity. Additional preferences may be selected; for example, if one wishes to attain better than 20% or better than 90% reduction in pain intensity, one may select the plasma concentrations indicated in FIG. 11 that correspond to the 20% or the 90% effectiveness levels, respectively.

As another example, but not as a limitation, parameters provided by the composite model are useful for selecting desirable higher levels of plasma concentrations of 6β-naltrexol. As one avenue, the EC51 parameter may be used in a fashion similar to the use of the EC52 parameter as described above.

A range of preferred dose amounts was calculated from the Emax composite model using EC20 derived from the graphic output and the sum of the EC52 plus the CV % obtained from the model. For example, a range of dose amounts is selected wherein the low point is the dose amount corresponding to the plasma concentration at EC20, and the high point is the dose amount corresponding to the plasma concentration that is the sum of the EC52 plus the CV % (133) obtained from the Emax composite model. By way of example, but not as a limitation, where the opioid antagonist naltrexone is provided in a dosing regimen that also includes 20 mg oxycodone, preferred dose amounts of opioid antagonist may comprise the range of from about 0.829 μg to about 2.37 μgs.

For a given opioid agonist that may be given in different dose amounts, it may be desirable to provide preferred concentrations or amounts of opioid antagonist. If the dosing regimen is to include 10 mg oxycodone (rather than 20 mg), an alternative preferred dose amounts may comprise the range of from about 0.415 μg to about 1.19 μgs. It is contemplated that, generally, a preferred dose amount may be adjusted in a proportionate manner to a change in oxycodone amount. If oxycodone amount is reduced or increased by a factor of 2, 4, or 8 (or other factor), the end points of the preferred range are each reduced or increased by a same factor (2, 4, or 8 or other factor)).

Accordingly, the plasma concentration-effect data set forth above for the subjects receiving the BID dosing regimen or the total plasma concentration-effect data (QID and BID dosing regimens) can be employed to select dose amounts of opioid antagonist to be administered. For example, the plasma concentration-effect data in Table 27, which relate to the plasma concentration-effect data from subjects receiving the BID dosing regimen, and the mathematical evaluation of the data using the Emax composite model, as exemplified in FIG. 12, may be employed to select dose amounts of opioid antagonist to be administered in a BID dosing regimen. Employing that data and the composite model, for a BID dosing regimen that includes 20 mg oxycodone, presently preferred dose amounts of opioid antagonist comprise from about 0.829 μg to about 2.37 μgs. Where the BID dosing regimen comprises other amounts of oxycodone per dose, exemplary dose amounts of opioid antagonist are contemplated:
1 mg oxycodone per dose: from about 0.041 μg to about 0.119 μg opioid antagonist per dose
2.5 mg oxycodone per dose: from about 0.103 μg to about 0.297 μg opioid antagonist per dose
5 mg oxycodone per dose: from about 0.207 μg to about 0.593 μg opioid antagonist per dose
10 mg oxycodone per dose: from about 0.415 μg to about 1.19 μgs opioid antagonist per dose
40 mg oxycodone per dose: from about 1.66 μgs to about 4.74 μgs opioid antagonist per dose
80 mg oxycodone per dose: from about 3.32 μgs to about 9.48 μgs opioid antagonist per dose
160 mg oxycodone per dose: from about 6.64 μgs to about 18.96 μgs opioid antagonist per dose
   Thus, for a BID dosing regimen that includes an amount of oxycodone, presently preferred dose amounts of opioid antagonist may comprise from about 0.041 μg to about 18.96 μgs.

As another example, the plasma concentration-effect data in Table 26, which relate to the total plasma concentration-effect data from subjects receiving the BID dosing regimen and subject receiving the QID dosing regimen, and the mathematical evaluation of the data using the composite Emax/Imax model, as exemplified in FIG. 11, may be employed to select preferred dose amounts of opioid antagonist more generally. For a dosing regimen that includes 20 mg oxycodone, presently preferred dose amounts of opioid antagonist comprise from about 0.830 μg to about 5.02 μgs. Where the dosing regimen comprises other amounts of oxycodone per dose, exemplary dose amounts of opioid antagonist are contemplated:
1 mg oxycodone per dose: from about 0.041 μg to about 0.252 μg opioid antagonist per dose
2.5 mg oxycodone per dose: from about 0.104 μg to about 0.63 μg opioid antagonist per dose
5 mg oxycodone per dose: from about 0.208 μg to about 1.26 μg opioid antagonist per dose
10 mg oxycodone per dose: from about 0.415 μg to about 2.51 μgs opioid antagonist per dose
40 mg oxycodone per dose: from about 1.66 μgs to about 10.0 μgs opioid antagonist per dose
80 mg oxycodone per dose: from about 3.32 μgs to about 20.1 μgs opioid antagonist per dose
160 mg oxycodone per dose: from about 6.64 μgs to about 40.2 μgs opioid antagonist per dose
   Thus, for a BID dosing regimen that includes an amount of oxycodone, presently preferred dose amounts of opioid antagonist may comprise from about 0.041 μg to about 40.2 μgs.

Furthermore, any of the foregoing ranges may be broadened by substituting the foregoing lower ends with a lower end of about 0.0002 μg, since dose amounts as low as about 0.0002 μg are presently contemplated. It was observed that the lower end of the ranges can approach zero based on the relatively low CV % s observed at the low end of the composite model (i.e., the values 132 and 151 for the BID and total (BID and QID) data sets, respectively). This indicates that even lower dose amounts of naltrexone and other opioid antagonists would be expected to be active, and dose amounts of about 0.0002 μg would be expected to be active albeit in a decreasing proportion of the population.

The present Example also provides preferred methods and materials comprising opioid antagonists other than naltrexone, such as naloxone and nalmefene. It is believed that, generally, the preferred dose amounts of naltrexone calculated above are useful for other opioid antagonists. Persons skilled in the field will recognize a particular opioid antagonist may have potency, bioavailability, metabolism, clearance, or other characteristics that suggest an adjustment to the dose amount, dosage form, or dosing regimen. For example, for opioid antagonists having reduce oral availability compared to naltrexone, it is contemplated that a higher oral dose amount will be provided, or that a more frequent dosing regimen will be employed, or that an intravenous dose will be provided, or some other adjustment will be made. Such adjustments are well within the ability of persons skill in the field.

As discussed above, methods and materials are provided for titrating an opioid antagonist administered to a human subject. By way of example, but not as a limitation, a suitable method comprises the steps of (a) administering an amount of an opioid antagonist and an amount of an opioid agonist to the subject, (b) measuring a plasma concentration in the subject of the opioid antagonist or a surrogate of the opioid antagonist, and (c) adjusting the amount of the opioid antagonist administered to the subject if the measured plasma concentration is outside a predetermined plasma concentration range. The predetermined plasma concentration range can be from concentrations predicted by a model of plasma concentration-effect relationship (e.g., the Emax composite model described above). The predetermined plasma concentration range can be the range predicted by the model to provide a reduction in pain intensity of about 20% or greater, alternatively about 50% or greater, alternatively about 90% or greater. The predetermined plasma concentration can be based on the plasma concentration-effect model shown in FIG. 11 or FIG. 12.

However, the present methods and materials for titrating an opioid antagonist administered to a human subject are not limited to the use of a composite model or to the use of predetermined plasma concentrations. By way of example, methods and materials of titrating an opioid antagonist administered to a human subject are provided, which comprise (a) administering an amount of an opioid antagonist and an amount of an opioid agonist to the subject, (b) assessing one or more symptoms or signs of an arthritic condition, inflammation associated with a chronic condition, or chronic pain, (c) measuring a plasma concentration in the subject of the opioid antagonist or a surrogate of the opioid antagonist, and (d) adjusting the amount of the opioid antagonist or the amount of the opioid agonist to the subject based on the measured plasma concentration. Step (d) may include comprises adjusting the amount of the opioid antagonist administered to the subject; alternatively or additionally, step (d) can comprises adjusting the amount of the opioid agonist administered to the subject.

As another example, methods and materials of titrating an opioid antagonist administered to a human subject are provided, which comprise (a) administering an amount of an opioid antagonist and an amount of an opioid agonist to the subject, (b) assessing one or more symptoms or signs of an arthritic condition, inflammation associated with a chronic condition, or chronic pain, and (c) adjusting the amount of the opioid antagonist administered to the subject if one or more of the assessed symptoms or signs are not alleviated to a desired extent. Step (c) can also comprise maintaining the amount of the opioid agonist administered to the subject. The method may also comprise the steps of (d) re-assessing one or more of the symptoms or signs after step (c), and (e) adjusting the amount of the opioid agonist if one or more of the assessed symptoms or signs are not alleviated to a desired extent.

In the titration methods and materials provided herein, it may be desirable to repeatedly administer the opioid antagonist such that a steady state is achieved before assessing one or more symptoms or signs of an arthritic condition, inflammation associated with a chronic condition, or chronic pain. The initial step of administering a first amount of an opioid antagonist and/or a first amount an opioid agonist can be repeated if the measured plasma concentration is within the predetermined plasma concentration range and/or if the assessed symptom(s) or sign(s) is alleviated to a desired extent.

In the titration methods and materials provided herein, it is contemplated that one or more of the assessed symptoms or signs may be pain, stiffness, and/or difficulty in physical function had by the subject, or measures of pain, stiffness and difficulty in physical function, such as the measures set forth in the WOMAC Osteoarthritis Index or one of its subscales. For example, a symptom or sign assessed for purposes of titration may be pain as measured as pain intensity. The pain intensity measurement may be attenuated as compared to a pain intensity baseline measurement of the subject. For example, the pain intensity measurement may be reduced by at least about 20%, alternatively at least about 50%, alternatively at least about 90%, compared to a pain intensity baseline measurement of the subject.

In the titration methods and materials provided herein, it is contemplated that a plasma concentration of the opioid antagonist or a surrogate of the opioid antagonist may be measured, and the amount of the opioid antagonist can be adjusted based in part on the measured plasma concentration. For example, the amount of the opioid antagonist administered to the subject is increased if the measured plasma concentration is lower than a predetermined plasma concentration value. As another example, the amount of the opioid antagonist administered to the subject is decreased in the measured plasma concentration is higher than a predetermined plasma concentration value. As yet another example, the amount of the opioid antagonist administered to the subject is maintained in the measured plasma concentration is within a predetermined plasma concentration range, and optionally the amount of the opioid agonist administered to the subject is increased.

While the foregoing generally preferred concentrations and amounts of opioid antagonists are contemplated for use with a wide variety of opioid agonists, it is contemplated that, for particular opioid agonists, particular concentrations and/or amounts may be selected based on the present disclosure. The foregoing generally preferred concentrations and amounts have been selected based on data from clinical studies employing the opioid antagonist naltrexone and the opioid agonist oxycodone, however they are also contemplated for use with a wide variety of opioid antagonists and opioid agonists.

### EXAMPLE 4

A clinical study was conducted as described in Example 1 (Part A) and data were obtained as described in Examples 1 and 2. Plasma samples from selected subjects in the clinical study were used to assay for the presence and concentration of selected cytokines.

Plasma samples were analyzed using a commercial cytokine assay from Pointilliste (www.pointilliste.com) to quantify the concentrations of IL2, IL4, IL5, IL6, IL10, IL13, GM-CSF, interferon and TNFα. Plasma samples were separately analyzed for IL1α and IL1β, which were quantitated using a conventional cytokine assay by Pointilliste. The cytokine assay for the quantitation of IL2, IL4, IL5, IL6, IL10, IL13, GM-CSF, interferon and TNFα employed, Pointilliste's Human Th1/Th2 Cytokine Canvas product which contains binding sites for each of these nine cytokines. The array pattern of cytokine antibodies printed in each well of a 96 well microtiter plate is shown in Table 28.

TABLE 28
Pattern of Human Th1/Th2 Cytokine Canvas

| | | | | |
|---|---|---|---|---|
| Alignment | Anti-IL2 | Anti-IL2 | Anti-IL13 | Anti-IL13 |
| marker | | | | |
| Negative | Anti-IL4 | Anti-IL4 | Anti-GM-CSF | Anti-GM-CSF |
| control | | | | |
| Negative | Anti-IL5 | Anti-IL5 | Anti-IFNγ | Anti-IFNγ |
| control | | | | |
| Reagent | Anti-IL6 | Anti-IL6 | Anti-TNFα | Anti-TNFα |
| Control 1 | | | | |
| Reagent | Anti-IL10 | Anti-IL10 | Negative | Alignment |
| Control 2 | | | control | marker |

Two measurements (duplicates) for each cytokine were possible for each plasma sample applied to a canvas, since the canvas has two binding sites for each cytokine.

Fifty-seven frozen human plasma samples containing EDTA as an anticoagulant were thawed on ice and transferred to a sterile 96-deep well polypropylene plate. The plate was centrifuged briefly at 4° C. to clarify the plasma. Aliquots of clarified plasma were removed for cytokine analysis, and the remaining samples in the 96-deep well polypropylene plate were stored at −80° C.

The aliquots of clarified plasma were transferred to sterile non-protein binding 96-well polypropylene plates to enable parallel processing of the samples. Each of the wells of these plates included a Pointilliste Human Th1/Th2 Cytokine Canvas as shown in Table 26. Two different 96 well plates were used, and each received a subsample of the aliquots at different dilutions. Two dilutions (1 in 1 and 1 in 10) of each sample were assayed on two separate human Th1/Th2 cytokine canvases. In order to quantify the cytokine concentrations, standard curves were generated for each dilution. A mixture of 9 cytokines was run on each of the canvases and used to calculate a standard curve, which was used to determine the amount of each cytokine in the samples. The standard curves were plotted with the signal intensity as a function of the cytokine concentration in ng/ml. A CCD camera was used with Pointilliste's Canvas Analysis Tools software to generate data corresponding to cytokine concentrations.

In addition to the assays using the Pointilliste Human Th1/Th2 Cytokine Canvas, conventional ELISA analysis was used to measure the concentrations of IL1α and IL1β in the plasma samples. The standard curves for IL1α and IL1β were also plotted with the signal intensity as a function of the cytokine concentration in ng/ml.

Tables 29 through 31 show measurements of cytokine concentrations (ng/ml) obtained as described herein and data calculated from those measured concentrations. In Tables 29 through 31, the following abbreviations are used: “OXY” refers to the treatment group receiving oxycodone QID as described in Example 1; “BID” refers to the treatment group receiving the combination drug of oxycodone and naltrexone BID as described in Example 1; “QID” refers to the treatment group receiving the combination drug of oxycodone and naltrexone QID as described in Example 1; “GM” refers to granulocyte/macrophage colony stimulating factor; “IFN” refers to interferon gamma; “TNF” refers to tumor necrosis factor alpha; “IL2” refers to interleukin 2; “IL4” refers to interleukin 4; “IL5” refers to interleukin 5; “IL6” refers to interleukin 6; “IL10” refers to interleukin 10; and “IL13” refers to interleukin 13.

Table 29 shows the individual cytokine measurements obtained from each sample as identified by sample identification number. Accordingly, Table 29 shows all the cytokine measurements that were obtained for each sample. Table 30 shows a compilation of the individual cytokine measurements obtained from the plasma samples. These measurements were used to determine the mean cytokine concentrations. The numbers of measurements for the various cytokines differ because different interferences affected samples and cytokine measurement within those samples differently.

TABLE 29
Individual Cytokine Concentrations (ng/ml)

| Requisition | | | | | | |
|---|---|---|---|---|---|---|
| # | IL2 | IL2 | IL4 | IL4 | IL5 | IL5 |
| 21896190 | 0.20055269 | 0.11856273 | | | | |
| 21896054 | | | | | | |
| 22982837 | | | 0.09166477 | 0.07630626 | | |
| 21366538 | 0.2226875 | 0.1560676 | | | | |
| 20889685 | | | 0.15974633 | 0.15681396 | | |
| 20468981 | 0.26894192 | 0.16131945 | | | | |
| 20871516 | 0.36578316 | 0.15581297 | | | | |
| 20996662 | | | | | | |
| 20322246 | 0.72330313 | 0.55323963 | 0.26509831 | 0.2369057 | | |
| 20893086 | 0.17732026 | 0.08939692 | | | | |
| 20904434 | | | | | | |
| 20902230 | | | | | | |
| 20982936 | | | | | | |
| 21641580 | 2.05566036 | 2.02979174 | 0.66585024 | 0.58444767 | | |
| 21641624 | | | | | | |
| 20997134 | | | | | | |
| 20?91744 | 0.18491683 | 0.1726987 | | | | |
| 20904803 | | | | | | |
| 20704612 | 0.47551031 | 0.40121823 | 0.13785932 | 0.1410828 | | |
| 20903725 | | | | | | |
| 20894411 | | | | | | |
| 21970721 | 0.21063224 | 0.15920151 | | | | |
| 21970800 | | | | | | |
| 20868748 | | | | | | |
| 21060022 | | | | | | |
| 21060145 | 0.13903841 | 0.07079245 | | | | |
| 21641760 | | | | | | |
| 21641850 | 1.12078831 | 0.89434998 | 0.28574849 | 0.24575749 | | |
| 21641793 | | | | | | |
| 22029704 | | | | | | |
| 22029884 | 0.20803264 | 0.12020192 | | | | |
| 22029895 | | | | | | |
| 20712802 | | | | | | |
| 20895614 | | | | | | |
| 20895647 | | | | | | |
| 21970710 | | | | | | |
| 20895120 | 0.32586262 | 0.15010384 | | | | |
| 20895030 | 0.65247635 | 0.48797657 | 0.16596928 | 0.16197549 | | |
| 20895197 | 1.81737173 | 1.58252682 | 0.47248956 | 0.46554916 | | |
| 20884555 | 0.17843304 | 0.09934644 | | | | |
| 20892464 | | | | | | |
| 21013356 | 0.16723079 | 0.0920016 | | | | |
| 22927195 | | | | | | |
| 23366766 | | | | | | |
| 22043250 | | | | | | |
| 22016002 | 1.0243213 | 0.88010212 | 0.49134739 | 0.56386303 | 0.48660202 | 0.48831413 |
| 22015910 | 0.47709418 | 0.36350033 | | | | |
| 22464955 | | | | | | |
| 23018163 | 0.16338282 | 0.03667778 | | | | |
| 21720868 | | | 0.09131771 | 0.09070153 | | |
| 21721037 | | | | | | |
| 22121010 | | | | | | |
| Requisition | | | | | | |
| # | IL6 | IL6 | IL10 | IL10 | IL13 | IL13 |
| 21896190 | 0.05665755 | 0.03555141 | | | 0.19392934 | 0.44115565 |
| 21896054 | 0.06451101 | 0.0692487 | | | | |
| 22982837 | | | | | | |
| 21366538 | 0.04990713 | 0.03994802 | | | 0.07605412 | 0.23363825 |
| 20889685 | 0.05213618 | 0.03663666 | | | 0.70581467 | 0.73173353 |
| 20468981 | | | | | | |
| 20871516 | | | | | 0.39807991 | 0.43517553 |
| 20996662 | | | | | | |
| 20322246 | 0.33537354 | 0.35986346 | 0.21636839 | 0.2438428 | 1.08994297 | 0.94101683 |
| 20893086 | | | | | 0.81325709 | 0.63358401 |
| 20904434 | | | | | 0.48282395 | 0.44626946 |
| 20902230 | | | | | | |
| 20982936 | | | | | 0.12980545 | 0.14265747 |
| 21641580 | 0.11742943 | 0.13925241 | 0.12089472 | 0.17918052 | 1.34132334 | 0.77623126 |
| 21641624 | | | 0.2371214 | 0.17262211 | 0.64922975 | 0.58858556 |
| 20997134 | | | | | | |
| 20?91744 | | | | | 0.47053745 | 0.33540459 |
| 20904803 | 0.09070808 | 0.00112223 | 0.12533194 | 0.37429311 | | |
| 20704612 | 0.07740341 | 0.07723647 | | | 0.62558119 | 0.33846604 |
| 20903725 | | | 0.07237012 | 0.22348702 | 0.1417683 | 0.10915494 |
| 20894411 | 0.0663709 | 0.06795795 | 0.0902624 | 0.22513845 | 0.1949292 | 0.17345874 |
| 21970721 | | | 0.04094618 | 0.08917843 | 0.50278268 | 0.45389581 |
| 21970800 | | | | | | |
| 20868748 | | | | | | |
| 21060022 | | | | | | |
| 21060145 | | | | | 0.48572734 | 0.46117658 |
| 21641760 | 0.14688034 | 0.13912545 | | | 0.064658 | 0.14512129 |
| 21641850 | 0.1582319 | 0.16709362 | | | 0.08465276 | 0.14293827 |
| 21641793 | | | | | 0.16898779 | 0.07300272 |
| 22029704 | 0.11310831 | 0.12393628 | | | 0.18947654 | 0.06004538 |
| 22029884 | 0.11153591 | 0.11069421 | 1.23635389 | 1.39363282 | 3.39229184 | 3.29071344 |
| 22029895 | 0.25877076 | 0.28127371 | | | 0.17262278 | 0.08433479 |
| 20712802 | | | | | 0.21364885 | 0.12981933 |
| 20895614 | | | | | | |
| 20895647 | 0.19964952 | 0.19868269 | | | 0.54685694 | 0.52753084 |
| 21970710 | | | | | 0.5223157 | 0.47200768 |
| 20895120 | | | 0.29585033 | 0.37571509 | 0.37002894 | 0.28470566 |
| 20895030 | 0.36904351 | 0.40419606 | 0.2361711 | 0.23885961 | 1.77426577 | 1.75789523 |
| 20895197 | 0.60404449 | 0.59568652 | 1.2602512 | 0.95982996 | 2.07889414 | 2.36158936 |
| 20884555 | | | | | 0.16535367 | 0.13963199 |
| 20892464 | | | | | | |
| 21013356 | | | | | 0.14153625 | 0.15088886 |
| 22927195 | 0.05790308 | 0.04453066 | | | | |
| 23366766 | 0.26943861 | 0.26152781 | | | | |
| 22043250 | 0.07944299 | 0.09620426 | | | | |
| 22016002 | 0.34897264 | 0.36998684 | 1.90278352 | 1.97097605 | 4.31719746 | 4.56515688 |
| 22015910 | 0.13710776 | 0.13450197 | 0.53936987 | 0.49835733 | 1.46693423 | 1.33230073 |
| 22464955 | 0.29919717 | 0.34984844 | 0.0579697 | 0.05035428 | 0.53207364 | 0.43591125 |
| 23018163 | 0.16754114 | 0.15867266 | 0.06037481 | 0.05236333 | 0.71059496 | 0.74330939 |
| 21720868 | 0.06949291 | 0.07330224 | | | | |
| 21721037 | | | | | | |
| 22121010 | 0.14418425 | 0.11755667 | 0.25846742 | 0.24059597 | 1.80730556 | 1.75018302 |
| Requisition | | | | | | |
| # | GM-CSF | GM-CSF | IFN gamma | IFN gamma | TNF alpha | TNF alpha |
| 21896190 | | | | | | |
| 21896054 | | | | | | |
| 22982837 | | | | | | |
| 21366538 | | | | | | |
| 20889685 | | | | | 0.07988409 | 0.05482446 |
| 20468981 | | | | | | |
| 20871516 | | | | | 0.0855576 | 0.06890438 |
| 20996662 | | | | | | |
| 20322246 | | | 0.0722552 | 0.07442144 | 0.20004415 | 0.14688815 |
| 20893086 | | | | | | |
| 20904434 | | | | | 0.10294933 | 0.16723002 |
| 20902230 | | | | | | |
| 20982936 | | | | | | |
| 21641580 | 0.33378408 | 0.37383059 | 0.2443029 | 0.29138781 | 0.42359475 | 0.38327461 |
| 21641624 | | | | | | |
| 20997134 | | | | | | |
| 20?91744 | | | | | | |
| 20904803 | | | | | | |
| 20704612 | | | | | | |
| 20903725 | | | | | | |
| 20894411 | | | | | | |
| 21970721 | | | | | | |
| 21970800 | | | | | | |
| 20868748 | | | | | | |
| 21060022 | | | | | | |
| 21060145 | | | | | | |
| 21641760 | | | | | | |
| 21641850 | | | 0.07860321 | 0.06975249 | 0.14351714 | 0.15694653 |
| 21641793 | | | | | | |
| 22029704 | | | | | | |
| 22029884 | 0.67155886 | 0.66705429 | | | 0.053116 | 0.05331531 |
| 20888717 | | | | | | |
| 22029895 | | | | | | |
| 20712802 | | | | | | |
| 20895614 | | | | | | |
| 20895647 | | | | | 0.05856579 | 0.05757872 |
| 21970710 | | | | | | |
| 20895120 | | | | | | |
| 20895030 | | | | | 0.37967017 | 0.35271469 |
| 20895197 | 0.60203722 | 0.53138737 | 0.21970779 | 0.24065142 | 0.51531889 | 0.51520632 |
| 20884555 | | | | | | |
| 20892464 | | | | | | |
| 21013356 | | | | | 0.05315699 | 0.06095267 |
| 22927195 | | | | | | |
| 23366766 | | | | | | |
| 22043250 | | | | | | |
| 22016002 | 1.41686033 | 1.40590028 | 0.13459554 | 0.12790808 | 0.27045659 | 0.26908761 |
| 22015910 | 0.26198022 | 0.2071141 | | | 0.12529323 | 0.13392305 |
| 22464955 | | | | | 0.07389075 | 0.0611985 |
| 23018163 | | | | | 0.05621241 | 0.06228325 |
| 21720868 | | | | | | |
| 21721037 | | | | | 0.0695864 | 0.05220257 |
| 22121010 | 0.23366778 | 0.20236439 | | | 0.04704521 | 0.05611238 |

TABLE 30
Cytokine Concentration Measurements Obtained
from Plasma Samples from Clinical Study
Cytokine Values

| OXY | BID | QID |
|---|---|---|
| IL2 | IL2 | IL2 |
| 0.0920016 | 0.07079245 | 0.03667778 |
| 0.12020192 | 0.08939692 | 0.1560676 |
| 0.15010384 | 0.09934644 | 0.15920151 |
| 0.16131945 | 0.11856273 | 0.16338282 |
| 0.16723079 | 0.13903841 | 0.21063224 |
| 0.20803264 | 0.15581297 | 0.2226875 |
| 0.26894192 | 0.1726987 | 0.48797657 |
| 0.32586262 | 0.17732026 | 0.65247635 |
| 0.40121823 | 0.17843304 | 0.88010212 |
| 0.47551031 | 0.18491683 | 0.89434998 |
| 0.55323963 | 0.20055269 | 1.0243213 |
| 0.72330313 | 0.36350033 | 1.12078831 |
| 1.58252682 | 0.36578316 | 2.02979174 |
| 1.81737173 | 0.47709418 | 2.05566036 |
| IL4 | IL4 | IL4 |
| 0.09070153 | 0.07630626 | 0.16197549 |
| 0.09131771 | 0.09166477 | 0.16596928 |
| 0.13785932 | 0.15681396 | 0.24575749 |
| 0.1410828 | 0.15974633 | 0.28574849 |
| 0.2369057 | | 0.49134739 |
| 0.26509831 | | 0.56386303 |
| 0.46554916 | | 0.58444767 |
| 0.47248956 | | 0.66585024 |
| IL5 | IL5 | IL5 |
| | | 0.48660202 |
| | | 0.48831413 |
| IL6 | IL6 | IL6 |
| 0.04453066 | 0.03555141 | 0.00112223 |
| 0.05790308 | 0.03663666 | 0.03994802 |
| 0.06949291 | 0.05213618 | 0.04990713 |
| 0.07330224 | 0.05665755 | 0.06451101 |
| 0.07723647 | 0.0663709 | 0.0692487 |
| 0.07740341 | 0.06795795 | 0.09070808 |
| 0.11069421 | 0.07944299 | 0.11742943 |
| 0.11153591 | 0.09620426 | 0.13925241 |
| 0.13912545 | 0.11310831 | 0.1582319 |
| 0.14688034 | 0.11755667 | 0.15867266 |
| 0.33537354 | 0.12393628 | 0.16709362 |
| 0.35986346 | 0.13450197 | 0.16754114 |
| 0.59568652 | 0.13710776 | 0.25877076 |
| 0.60404449 | 0.14418425 | 0.26152781 |
| | 0.19868269 | 0.26943861 |
| | 0.19964952 | 0.28127371 |
| | 0.29919717 | 0.34897264 |
| | 0.34984844 | 0.36904351 |
| | | 0.36998684 |
| | | 0.40419606 |
| IL10 | IL10 | IL10 |
| 0.07237012 | 0.05035428 | 0.04094618 |
| 0.21636839 | 0.0579697 | 0.05236333 |
| 0.22348702 | 0.0902624 | 0.06037481 |
| 0.2438428 | 0.22513845 | 0.08917843 |
| 0.29585033 | 0.24059597 | 0.12089472 |
| 0.37571509 | 0.25846742 | 0.12533194 |
| 0.95982996 | 0.49835733 | 0.17262211 |
| 1.23635389 | 0.53936987 | 0.17918052 |
| 1.2602512 | | 0.2361711 |
| 1.39363282 | | 0.2371214 |
| | | 0.23885961 |
| | | 0.37429311 |
| | | 1.90278352 |
| | | 1.97097605 |
| IL13 | IL13 | IL13 |
| 0.064658 | 0.06004538 | 0.07605412 |
| 0.07300272 | 0.12981933 | 0.08433479 |
| 0.10915494 | 0.13963199 | 0.08465276 |
| 0.14153625 | 0.16535367 | 0.12980545 |
| 0.1417683 | 0.17345874 | 0.14265747 |
| 0.14512129 | 0.18947654 | 0.14293827 |
| 0.15088886 | 0.19392934 | 0.17262278 |
| 0.16898779 | 0.1949292 | 0.23363825 |
| 0.28470566 | 0.21364885 | 0.44626946 |
| 0.33846604 | 0.33540459 | 0.45389581 |
| 0.37002894 | 0.39807991 | 0.48282395 |
| 0.62558119 | 0.43517553 | 0.50278268 |
| 0.94101683 | 0.43591125 | 0.58858556 |
| 1.08994297 | 0.44115565 | 0.64922975 |
| IL13 | IL13 | IL13 |
| 2.07889414 | 0.46117658 | 0.71059496 |
| 2.36158936 | 0.47053745 | 0.74330939 |
| 3.29071344 | 0.47200768 | 0.77623126 |
| 3.39229184 | 0.48572734 | 1.34132334 |
| | 0.5223157 | 1.75789523 |
| | 0.52753084 | 1.77426577 |
| | 0.53207364 | 4.31719746 |
| | 0.54685694 | 4.56515688 |
| | 0.63358401 | |
| | 0.70581467 | |
| | 0.73173353 | |
| | 0.81325709 | |
| | 1.33230073 | |
| | 1.46693423 | |
| | 1.75018302 | |
| | 1.80730556 | |
| GM | GM | GM |
| 0.53138737 | 0.20236439 | 0.33378408 |
| 0.60203722 | 0.2071141 | 0.37383059 |
| 0.66705429 | 0.23366778 | 1.40590028 |
| 0.67155886 | 0.26198022 | 1.41686033 |
| IFN | IFN | IFN |
| 0.0722552 | | 0.06975249 |
| 0.07442144 | | 0.07860321 |
| 0.21970779 | | 0.12790808 |
| 0.24065142 | | 0.13459554 |
| | | 0.2443029 |
| | | 0.29138781 |
| TNF | TNF | TNF |
| 0.053116 | 0.04704521 | 0.05621241 |
| 0.05315699 | 0.05220257 | 0.06228325 |
| 0.05331531 | 0.05482446 | 0.10294933 |
| 0.06095267 | 0.05611238 | 0.14351714 |
| 0.14688815 | 0.05757872 | 0.15694653 |
| 0.20004415 | 0.05856579 | 0.16723002 |
| 0.51520632 | 0.0611985 | 0.26908761 |
| 0.51531889 | 0.06890438 | 0.27045659 |
| | 0.0695864 | 0.35271469 |
| | 0.07389075 | 0.37967017 |
| | 0.07988409 | 0.38327461 |
| | 0.0855576 | 0.42359475 |
| | 0.12529323 | |
| | 0.13392305 | |

As indicated by the missing values in Table 29 and the different number of measurements in Table 30 for the various cytokines, the cytokine assay did not provide measurements of all nine cytokines for each sample. Many cytokine measurements were not obtained due to one or more interferences with the detection mechanism of the assay. The missing values are attributed to random occurrences of high background, excess heme, lipolysis, desiccation, and the lowest level of quantification (LLOQ) for IL1. However, the missing values for cytokine concentrations occurred randomly among the subjects, and the random occurrence of missing values is believed to not interfere with the accumulation of data. Accordingly the measurements which were obtained from the assay are believed to be meaningful.

Table 30 shows the differences in cytokine levels between the different treatment groups (OXY, BID and QID) in the clinical study. Table 31 the means for each treatment group of the plasma concentrations of the various cytokines, along with the standard deviation for the measurements within the treatment groups. The mean values for the cytokine concentrations detected for each plasma sample analyzed from the various treatment groups is set forth along with the standard deviation. The mean and standard deviation values were calculated using the duplicate values obtained from various plasma samples.

TABLE 31
Mean of Cytokine Concentrations (ng/ml) from Random Sample of Subjects

| | OXY | BID | QID | OXY | BID | QID | OXY | BID | QID |
|---|---|---|---|---|---|---|---|---|---|
| | IL2 | IL2 | IL2 | IL4 | IL4 | IL4 | IL5 | IL5 | IL5 |
| N | 14 | 14 | 14 | 8 | 4 | 8 | 0 | 0 | 2 |
| Mean | 0.503 | 0.12 | 0.721 | 0.238 | 0.211 | 0.396 | | | 0.487 |
| Std Dev | 0.54 | 0.119 | 0.667 | 0.156 | 0.043 | 0.203 | | | 0.001 |

| | OXY | BID | QID | OXY | BID | QID | OXY | BID | QID |
|---|---|---|---|---|---|---|---|---|---|
| | IL6 | IL6 | IL6 | IL10 | IL10 | IL10 | IL13 | IL13 | IL13 |
| N | 14 | 18 | 20 | 10 | 8 | 14 | 18 | 30 | 22 |
| Mean | 0.2 | 0.128 | 0.189 | 0.628 | 0.245 | 0.414 | 0.876 | 0.559 | 0.917 |
| Std Dev | 0.194 | 0.087 | 0.123 | 0.52 | 0.188 | 0.652 | 1.121 | 0.459 | 1.244 |

| | OXY | BID | QID | OXY | BID | QID | OXY | BID | QID |
|---|---|---|---|---|---|---|---|---|---|
| | GM | GM | GM | IFN | IFN | IFN | TNF | TNF | TNF |
| N | 4 | 4 | 4 | 4 | 0 | 6 | 8 | 14 | 12 |
| Mean | 0.616 | 0.226 | 0.883 | 0.152 | | 0.158 | 0.2 | 0.073 | 0.231 |
| Std Dev | 0.066 | 0.027 | 0.611 | 0.091 | | 0.09 | 0.2-2 | 0.026 | 0.132 |

These data indicate that methods and materials as described herein for the treatment of arthritic conditions, inflammation associated with a chronic condition, and/or chronic pain, including pain in conjunction or associated with arthritic conditions or inflammation, are useful to decrease the plasma concentration of various proinflammatory cytokines. These data also indicate that cytokines are appropriate biomarkers, including for the monitoring, detection, diagnosis and/or treatment of arthritic conditions, inflammation associated with a chronic condition and/or chronic pain. Such biomarkers are useful to detect anti-inflammatory activity or other effects of the present methods and materials. Biomarkers, such as cytokines, are of interest to the pharmaceutical industry for various uses, including, for example, to determine potential activity of drugs in clinical development.

### EXAMPLE 5

Solid oral dosage forms comprising opioid agonists and/or opioid antagonists can be prepared by a variety of processes well-known to those skilled in the art. For example, methods and materials as described in U.S. Patent Application Publication No. 2003/0191147 (previously incorporated by reference herein) and WO 01/85257 (PCT/US01/14377) are useful in preparing dosage forms comprising opioid agonists and/or opioid antagonists, including wherein the dosage form comprises amounts of opioid antagonists of 1 mg or less. As another example, solid oral dosage forms comprising oxycodone hydrochloride (OXY) and naltrexone hydrochloride (NTX) are prepared as described herein. For clinical studies as described in Example 1, tablets having different amounts of oxycodone were manufactured, though the amount of naltrexone was the same (0.001 mg) among the tablets of different strength.

Tablet formulations containing oxycodone HCl at various dose levels (2.5, 5, 7.5, 10, 15 and 20 mg/tablet) and low-dose naltrexone HCl (0.001 mg) were prepared. Four matching active controls of oxycodone HCl tablets at various strengths (2.5, 5, 7.5, and 10 mg/tablet) and a matching placebo tablet were also prepared.

A constant weight series based on a common formulation is followed in the manufacture of oxycodone HCl/naltrexone HCl tablets, oxycodcone HCl tablets, and placebo tablets. Differences in the mass of the active pharmaceutical ingredient (API) in the various tablet dosage strengths (in this case oxycodone) are compensated for by adjusting the amount of lactose monohydrate to achieve a consistent mass among all active and placebo tablets.

The components, pharmaceutical grade, and function of each component used to make oxycodone HCl/naltrexone HCl tablets and oxycodone HCl tablets are provided in Table 32 below. Except for the Opadry® film coatings, the components used in the tablet dosage forms are compendial in the current USP/NF.

TABLE 32
Components for Oxycodone HCl/Naltrexone
HCl Tablets and Oxycodone HCl Tablets

| Component | Function |
|---|---|
| Oxycodone HCl, USP | Active pharmaceutical |
| | ingredient |
| Naltrexone HCl, USP* | Active pharmaceutical |
| | ingredient |
| Lactose, Monohydrate, NF | Diluent |
| Hydroxypropyl Methylcellulose, USP | Binder |
| Citric Acid, Anhydrous, USP | Acidifier for pH adjustment |
| Sodium Hydroxide, NF | Alkalizer for pH adjustment |
| Low-Substitured Hydroxypropyl | Disintegrant |
| Cellulose, NF | |
| Magnesium Stearate, NF | Lubricant |
| Talc, USP (Hydrous Magnesium | Glidant |
| Silicate) | |
| Water** for Injection, USP | Processing Solvent |
| Opadry ® Clear | Base Coat |
| Opadry ® II Yellow | Aesthetic Color Coat |

*Naltrexone HCl not present in oxycodone HCl tablets.
**Removed during processing

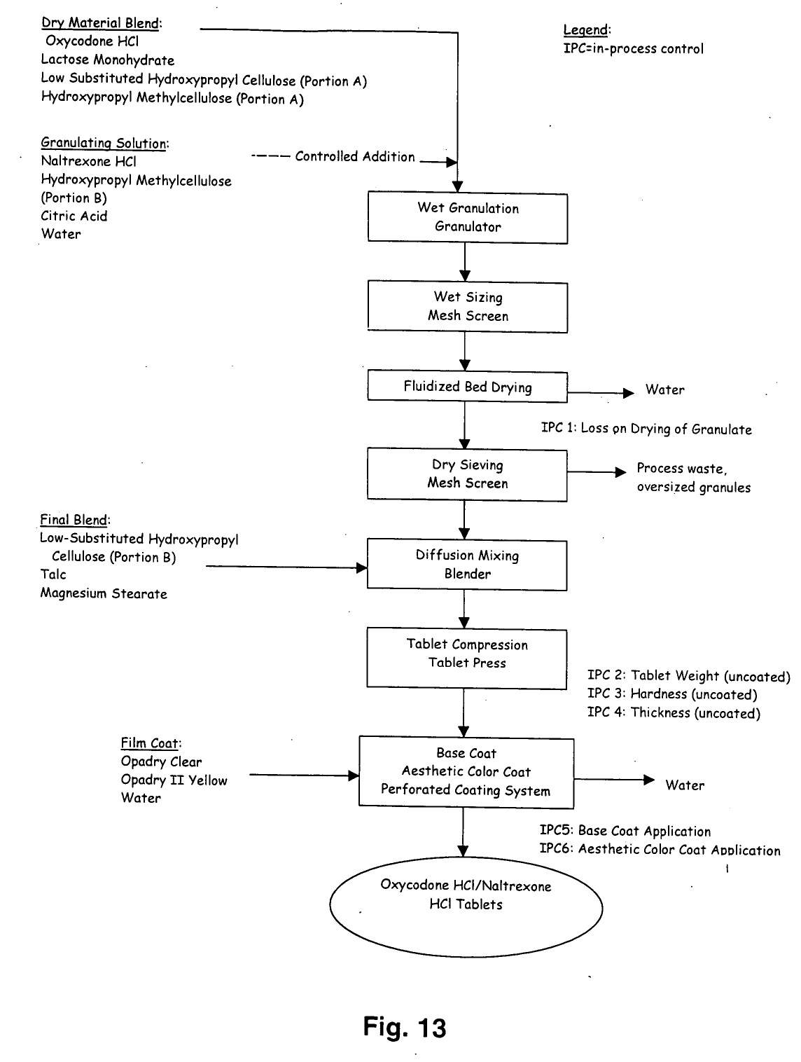

The following steps were used to prepare tablets comprising oxycodone and naltrexone. These steps as well as in-process controls (IPC) are summarized in the flowchart of FIG. 13.

Oxycodone HCl, lactose monohydrate, low-substituted hydroxypropyl cellulose (Portion A), and hydroxypropyl methylcellulose (Portion A) were dry blended in a granulator. This dry material blend was granulated in a wet granulation step with an aqueous solution of naltrexone HCl, citric acid, and hydroxypropyl methylcellulose solution (pH at 3.5) (Portion B). More water was added if needed to obtain a satisfactory granulation. The wet granulation was sieved in a wet sizing step through a mesh screen and dried in a fluidized bed to an endpoint moisture content of not more than 3 percent determined by a Loss on Drying (LOD) measurement.

The dried granulation was sieved through a mesh screen in a dry sieving step. A portion of the dried granulate approximately equal to the balance of formulation components was reserved. The remaining granulate was added to a V-blender.

Each of the three components (low-substituted hydroxypropyl cellulose (Portion B), talc, and magnesium stearate) were combined with an approximately equal portion of the reserved dry granulation to form intermediate mixtures. Each intermediate mixture was sequentially added through a mesh screen and into the V-blender. The granulation was blended after each addition to achieve uniformity.

The blended granulation was compressed into tablets on a rotary tablet press. Tablets had a mean weight of about 200 mg. (approximate range 190 mg to 210 mg), mean hardness in the range of about 5 kp to 8 kp (approximate range 4 kp to 10 kp) and mean thickness of 4.3 to 4.7 mm.

Tablets were film coated in a perforated pan that included application of a clear base coating followed by an aesthetic color coating. A commercially available clear coating (Colorcon-Opadry Clear) was applied to achieve an average coating weight of 2±0.4 mg per tablet. A commercially available color coating (Colorcon-Opadry II Yellow) was applied to achieve an average coating weight of approximately 8±1 mg per tablet.

The amounts of active ingredients and excipients in various tablets of different strengths are set forth in Tables 33 through 38.

Table 33 sets forth the composition of exemplary 2.5 mg strength tablets (tablets comprising 2.5 mg oxycodone HCl and 0.001 mg naltrexone hydrochloride).

TABLE 33
Oxycodone HCl 2.5 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 2.50 | 8.3 |
| Naltrexone HCl, USP | 0.001 | 0.0033** |
| Lactose, Monohydrate, NF | 169.80 | 560.3 |
| Hydroxypropyl Methylcellulose, | 2.82 | 9.3** |
| USP | | |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium | 6.00 | 19.8 |
| Silicate) | | |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic | 8.00 | 26.4** |
| Color Coat) | | |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 34 sets forth the composition of exemplary 5 mg strength tablets (tablets comprising 5 mg oxycodone HCl and 0.001 mg naltrexone hydrochloride).

TABLE 34
Oxycodone HCl 5 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 5.00 | 16.5 |
| Naltrexone HCl, USP | 0.001 | 0.0033** |
| Lactose, Monohydrate, NF | 167.30 | 552.1 |
| Hydroxypropyl Methylcellulose, | 2.82 | 9.3** |
| USP | | |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium | 6.00 | 19.8 |
| Silicate) | | |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic | 8.00 | 26.4** |
| Color Coat) | | |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 35 sets forth the composition of exemplary 7.5 mg strength tablets (tablets comprising 7.5 mg oxycodone HCl and 0.001 mg naltrexone hydrochloride).

TABLE 35
Oxycodone HCl 7.50 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 7.50 | 24.8 |
| Naltrexone HCl, USP | 0.001 | 0.0033** |
| Lactose, Monohydrate, NF | 164.80 | 543.8 |
| Hydroxypropyl Methylcellulose, | 2.82 | 9.3** |
| USP | | |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium | 6.00 | 19.8 |
| Silicate) | | |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic | 8.00 | 26.4** |
| Color Coat) | | |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 36 sets forth the composition of exemplary 10 mg strength tablets (tablets comprising 10 mg oxycodone HCl and 0.001 mg naltrexone hydrochloride).

TABLE 36
Oxycodone HCl 10 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 10.00 | 33.0 |
| Naltrexone HCl, USP | 0.001 | 0.0033** |
| Lactose, Monohydrate, NF | 162.30 | 535.6 |
| Hydroxypropyl Methylcellulose, | | |
| USP | | |
| Citric Acid, Anhydrous, USP | | |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium | 6.00 | 19.8 |
| Silicate) | | |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic | 8.00 | 26.4** |
| Color Coat) | | |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 37 sets forth the composition of exemplary 15 mg strength tablets (tablets comprising 15 mg oxycodone HCl and 0.001 mg naltrexone hydrochloride).

TABLE 37
Oxycodone HCl 15 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 15.00 | 49.5 |
| Naltrexone HCl, USP | 0.001 | 0.0033** |
| Lactose, Monohydrate, NF | 157.30 | 519.1 |
| Hydroxypropyl Methylcellulose, | 2.82 | 9.3** |
| USP | | |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium | 6.00 | 19.8 |
| Silicate) | | |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic | 8.00 | 26.4** |
| Color Coat) | | |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 38 sets forth the composition of exemplary 20 mg strength tablets (tablets comprising 20 mg oxycodone HCl and 0.001 mg naltrexone hydrochloride).

TABLE 38
Oxycodone HCl 20 mg/Naltrexone HCl 0 001 mg Tablets

| | Quantity per | Quantity per |
|---|---|---|
| Component | Tablet (mg) | Batch (g) |
| Tablet Core: | | |
| Oxycodone HCl, USP | 20.00 | 66.0 |
| Naltrexone HCl, USP | 0.001 | 0.0033** |
| Lactose, Monohydrate, NF | 152.30 | 502.6 |
| Hydroxypropyl Methylcellulose, | 2.82 | 9.3** |
| USP | | |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium | 6.00 | 19.8 |
| Silicate) | | |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic | 8.00 | 26.4** |
| Color Coat) | | |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Clinical supplies of oxycodone HCl/naltrexone HCl tablets, oxycodone HCl tablets, or placebo tablets were packaged in plastic film blister packs with foil backing. The blister packs were placed inside a foil/foil pouch with a silica gel desiccant to assure that products conform to specifications while in use.

An advantage of dosage forms prepared as referenced and described in this example, including tablets made by the procedure described above and summarized in FIG. 13 is that undesirable binding of the opioid antagonist to the excipients is essentially avoided. It was previously noted that some opioid antagonists undesirably bind significantly to certain pharmaceutical excipients in an environment of use (see, e.g., WO 01/85257 (PCT/US01/14377) and U.S. Patent Application Publication No. 2003/0191147). Undesirable binding generally causes an incomplete amount of the opioid antagonist to be released from a dosage form, within a particular time allotted for release in a dissolution test or in clinical use. For example, as described herein, the use of an acidic pH during the wet granulating step was advantageous with respect to avoiding undesirable binding. As described above, the wet granulation step employed a granulation solution having a pH adjusted to 3.5 with citric acid. The tablets manufactured by this manufacturing process did not exhibit undesirable binding of the opioid antagonist and the excipients to a significant degree. Accordingly, some embodiments of the present methods and materials include steps or excipients which reduce or minimize undesirable binding of opioid antagonist and one or more pharmaceutical excipients, so that such excipients do not bind the opioid antagonist to a significant degree in an environment of use.

While the invention will be described in connection with one or more embodiments, it will be understood that the invention is not limited to those embodiments. On the contrary, the invention includes all alternatives, modification, and equivalents as may be included within the spirit and scope of the appended claims.

1. A method for treating an arthritic condition in a human subject comprising administering to the subject an opioid agonist and an opioid antagonist, wherein one or more symptoms or signs associated with the arthritic condition are alleviated.

2-3. (canceled)

4. The method of claim 1, wherein the arthritic condition is associated with a joint, hip, knee, back, neck, or lower back of the subject.

5. The method of claim 1, wherein the symptom or sign is pain.

6. The method of claim 5, wherein the pain is measured as pain intensity.

7-14. (canceled)

15. The method of claim 1, wherein the symptom or sign is stiffness.

16-20. (canceled)

21. The method of claim 1, wherein the symptom or sign is difficulty in physical function had by the subject.

22-26. (canceled)

27. The method of claim 1, wherein the total score of the subject on the WOMAC Osteoarthritis Index is attenuated.

28-33. (canceled)

34. A method for treating inflammation associated with a chronic condition in a human subject comprising administering to the subject an opioid agonist and an opioid antagonist, wherein one or more signs or symptoms associated with the inflammation is alleviated.

35. The method of claim 34, wherein the chronic condition is an arthritic condition.

36-37. (canceled)

38. The method of claim 35, wherein the arthritic condition is associated with a joint, hip, knee, back, neck, or lower back of the subject.

39. The method of claim 34, wherein the symptom or sign is pain.

40. The method of claim 39, wherein the pain is measured as pain intensity.

41-54. (canceled)

55. The method of claim 34, wherein the symptom or sign is difficulty in physical function had by the subject.

56-60. (canceled)

61. The method of claim 34, wherein the total score of the subject on the WOMAC Osteoarthritis index is attenuated.

62-67. (canceled)

68. A method for treating chronic pain comprising administering to a human subject with chronic pain an opioid agonist and an opioid antagonist, wherein the chronic pain is attenuated.

69. The method of claim 68, wherein the chronic pain is associated with an arthritic condition.

70-71. (canceled)

72. The method of claim 68, wherein the chronic pain is associated with a joint, hip, knee, back, neck, or lower back of the subject.

73. The method of claim 68 wherein the pain is measured as pain intensity.

74-81. (canceled)

82. The method of claim 1, 34 or 68 further comprising administering to the subject an additional therapeutic agent that is a non-steroidal anti-inflammatory drug, cytokine inhibitor, corticosteroid, anti-rheumatic drug, anticonvulsant agent, tricyclic antidepressant agent, anti-dynorphin agent, or glutamate receptor antagonist agent.

83. The method of claim 82, wherein the additional therapeutic agent is a TNF-α inhibitor, corticosteroid, anti-rheumatic drug, non-steroidal anti-inflammatory drug, celecoxib, ropecoxib, valdecoxib, etanercept, infiximab, anti-TNF-α, D2E7 human Mab, CDP-870, CDP-571, humicade, PEGylated soluble TNF-α Receptor-1, TBP-1, PASSTNF-alpha, AGT-1, ienercept, CytoTAB, TACE, small molecule TNF mRNA synthesis inhibitor, PEGylated p75TNFR Fc mutein (Immunex), TNF-α antisense inhibitor, methotrexate, leflunomide, D-Penicillamine, sulfasalazine, a gold composition, minocycline, azathioprine, hydroxychloroquine, an antimalarial drug, cyclosporine, or a biologic agent that designed to either inhibit or supplement a cytokine.

84. The method of claims 1, 34 or 68, wherein the agonist, the antagonist, or both the agonist and the antagonist is administered no more than twice in a 24-hour period.

85. The method of claims 1, 34 or 68, wherein the agonist, the antagonist, or both the agonist and the antagonist is administered no more than once in a 24-hour period.

86. The method of claims 1, 34 or 68, wherein the amount of the antagonist administered in a 24-hour period is less than 0.004 mg.

87. The method of claims 1, 34 or 68, wherein the amount of the antagonist administered in a 24-hour period is 0.002 mg or less.

88. The method of claims 1, 34 or 68, wherein the antagonist, the agonist, or both the antagonist and the agonist is administered in an oral dosage form.

89. The method of claim 88, wherein the oral dosage form is a solid oral dosage form or a liquid oral dosage form.

90. The method of claims 1, 34 or 68, wherein the agonist is oxycodone, oxymorphone, hydrocodone, hydromorphone, or morphine.

91. The method of claims 1, 34 or 68, wherein the antagonist is naltrexone, nalmefene, or naloxone.

92. The method of claims 1, 34 or 68, wherein the mode of administration is oral, intravenous, intrathecal, epidural, intramuscular, subcutaneous, perineural, intradermal, topical, or transcutaneous.

93. The method of claims 1, 34 or 68, wherein the agonist is selected from the group consisting of oxycodone, oxymorphone, hydrocodone, hydromorphone, and morphine, and the antagonist is naltrexone.

94. The method of claims 1, 34 or 68, wherein the agonist is oxycodone and the antagonist is naltrexone.

95. The method of claims 1, 34 or 68, wherein the amount of the agonist is from about 2.5 mg to about 160 mg.

96. The method of claims 1, 34 or 68, wherein the amount of the antagonist is from about 0.0001 mg to less than about 0.004 mg.

97. The method of claims 1, 34 or 68, wherein the amount of the agonist is selected from the group consisting of the amounts: about 2.5 mg, about 5 mg, about 10 mg, about 15 mg, about 20 mg, about 30 mg, about 40 mg, about 80 mg, about 160 mg, and about 320 mg.

98. The method of claims 1, 34 or 68, wherein the amount of the antagonist administered is at least about 1250 fold less than the amount of the agonist administered.

99. The method of claims 1, 34 or 68, wherein the amount of the antagonist administered is at most about 1,600,000 fold less than the amount of the agonist administered.

100. The method of claims 1, 34 or 68, wherein the antagonist, the agonist, or both the antagonist and the agonist are administered once daily.

101. The method of claims 1, 34 or 68, wherein the antagonist, the agonist, or both the antagonist and the agonist are administered twice daily.

102-103. (canceled)

104. A method for treating an arthritic condition comprising administering to a human subject a dosage form for oral administration, wherein the dosage form comprises no more than about 80.4 pgs, about 40.2 μgs, about 20 μgs, about 10 μgs, about 5 μgs, about 2.5 μgs, about 1.2 μgs, about 0.6 μg, about 0.3 μg, or about 0.12 μg of the opioid antagonist.

105. The method of claim 104, wherein the dosage form comprises at least about 0.0002 μg, about 0.1 μg, about 0.2 μg, about 0.4 μg, about 0.8 μg, about 1.6 μg, about 3.3 μg, or about 6.6 μg of the opioid antagonist.

106. A method for treating inflammation associated with a chronic condition comprising administering to a human subject a dosage form for oral administration, wherein the dosage form comprises no more than about 80.4 μgs, about 40.2 μgs, about 20 μgs, about 10 μgs, about 5 μgs, about 2.5 μgs, about 1.2 μgs, about 0.6 μg, about 0.3 μg, or about 0.12 μg of the opioid antagonist.

107. The method of claim 106, wherein the dosage form comprises at least about 0.0002 μg, about 0.1 μg, about 0.2 μg, about 0.4 μg, about 0.8 μg, about 1.6 μg, about 3.3 μg, or about 6.6 μg of the opioid antagonist.

108. A method for treating chronic pain comprising administering to a human subject with chronic pain a dosage form for oral administration, wherein the dosage form comprises no more than about 80.4 μgs, about 40.2 μgs, about 20 μgs, about 10 μgs, about 5 μgs, about 2.5 μgs, about 1.2 μgs, about 0.6 μg, about 0.3 μg, or about 0.12 μg of the opioid antagonist.

109. The method of claim 108, wherein the dosage form comprises at least about 0.0002 μg, about 0.1 μg, about 0.2 μg, about 0.4 μg, about 0.8 μg, about 1.6 μg, about 3.3 μg, or about 6.6 μg of the opioid antagonist.

110-112. (canceled)

113. A method for treating chronic pain comprising in a human subject comprising:
administering an opioid agonist to the human subject; and
administering an opioid antagonist to the human subject no more than twice-a-day.

114. The method of claim 113, wherein the opioid antagonist is administered twice-a-day.

115. The method of claim 113, wherein the opioid antagonist is administered once-a-day.

116. The method of claim 113, wherein the opioid antagonist is orally administered.

117. The method of claim 113, wherein the opioid agonist is orally administered.

118. The method of claim 113, wherein the opioid agonist and the opioid antagonist are each orally administered in a dose amount twice-a-day.

119. The method of claim 118, wherein the dose amount of the opioid agonist is about 1 mg and the dose amount of opioid antagonist is from about 0.041 μg to about 0.119 μg, or the dose amount of the opioid agonist is about 2.5 mg and the dose amount of opioid antagonist is from about 0.103 μg to about 0.297 μg, or the dose amount of the opioid agonist is about 5 mg and the dose amount of opioid antagonist is from about 0.207 μg to about 0.593 μg, or the dose amount of the opioid agonist is about 10 mg and the dose amount of opioid antagonist is from about 0.415 μg to about 1.19 μgs, or the dose amount of the opioid agonist is about 20 mg and the dose amount of the opioid antagonist is from about 0.829 μg to about 2.37 μgs, or the dose amount of the opioid agonist is about 40 mg and the dose amount of the opioid antagonist is from about 1.66 μgs to about 4.74 μgs, or the dose amount of the opioid agonist is about 80 mg and the dose amount of the opioid antagonist is from about 3.32 μgs to about 9.48 μgs, or the dose amount of the opioid agonist is about 160 mg and the dose amount of the opioid antagonist is from about 6.64 μgs to about 18.96 μgs, or or the dose amount of the opioid agonist is about 320 mg and the dose amount of the opioid antagonist is from about 13.28 μgs to about 37.92 μgs.

120-168. (canceled)
